(19) United States
(12) Patent Application Publication
(10) Pub. No.: US 2024/0360089 A1
(43) Pub. Date: Oct. 31, 2024

(54) Metabolically Stable HDAC Inhibitors with Trifluoromethylpyruvamide as Metal-Binding Group

(71) Applicant: The University of Toledo, Toledo, OH (US)

(72) Inventors: Endri Karaj, Toledo, OH (US); Liyanaaratchige V. Tillekeratne, Toledo, OH (US); Riddhidev Banerjee, Toledo, OH (US); William R. Taylor, Toledo, OH (US)

(73) Assignee: The University of Toledo, Toledo, OH (US)

(21) Appl. No.: 18/585,551

(22) Filed: Feb. 23, 2024

Related U.S. Application Data
(60) Provisional application No. 63/461,684, filed on Apr. 25, 2023.

(51) Int. Cl. C07D 277/82; A61P 35/00; C07D 277/54

(52) U.S. Cl. CPC C07D 277/82; A61P 35/00; C07D 277/54

(57) ABSTRACT

HDAC inhibitor compounds having trifluoromethylpyruvamides or hydrates thereof, and methods of making and using the same, are described. The HDAC inhibitor compounds are useful for inhibiting HDAC activity and inhibiting growth of cancer cells.

### RELATED APPLICATIONS

This application claims priority to U.S. Provisional Application No. 63/461,684 filed under 35 U.S.C. § 111(b) on Apr. 25, 2023, the disclosure of which is incorporated herein by reference in its entirety for all purposes.

### STATEMENT REGARDING FEDERALLY SPONSORED RESEARCH

This invention was made with government support under Grant Numbers R15GM120712 and R15CA213185 awarded by the National Institutes of Health. The government has certain rights in this invention.

### BACKGROUND

Cancer is the second largest cause of death in the United States. Despite improvement in treatment regimens, problems such as lack of target specificity and off-target toxicity persist. Targeted drug therapy is a possible way to counter these problems. Enzymes responsible for epigenetic regulation of gene expression provide several targets for rational design of anti-cancer agents. Epigenetic regulation includes DNA methylation and histone acetylation/deacetylation, which do not change the genetic sequence, but alter the rate of transcription of genes affected by these modifications.

Histone acetylation and deacetylation are regulated by two families of enzymes, histone acetyltransferases (HATs) and histone deacetylases (HDACs), respectively. HDACs play a key role in regulating gene expression by removing acetyl groups from histones, which are proteins that help package and organize DNA in the nucleus of cells. Deacetylation of histones can reduce the expression of tumor suppressor genes, and their hyperacetylation can lead to their induction, leading to apoptosis. HDACs also deacetylate non-histone proteins such as α-tubulin, which are involved in cancer cell migration and proliferation. Hence, selective targeting of HDACs has been studied extensively. By inhibiting HDAC activity, HDAC inhibitors can increase the acetylation of histones, which in turn can lead to changes in gene expression.

HDACs consist of zinc-dependent (Class I, II, and IV) and NAD⁺ dependent (Class III) enzymes. Class I and II HDACs can be inhibited by small molecules containing Zinc-Binding Groups (ZBGs), which bind to a Zn²⁺ ion in the enzyme active site via mono or bidentate complexation, which is reversible in nature. The ZBG is a chemical moiety that binds to the zinc ion in the active site of the HDAC enzyme, thereby inhibiting its activity. The choice of ZBG can affect the potency, selectivity, and pharmacokinetic properties of an HDAC inhibitor. The ZBG typically includes a group such as a carboxylate, a hydroxamate, or a thiol. Of the four HDAC inhibitors approved by the US FDA for clinical use, vorinostat (SAHA) and belinostat (Beleodaq) approved for T-cell lymphoma and panobinostat (Farydak) approved for multiple myeloma (FIG. 1) are hydroxamates, while romidepsin (Istodax) approved for T-cell lymphoma has a thiol ZBG. Chidamide (Epidaza, FIG. 1) is a benzamide that is in clinical trials in USA and in clinical use in China. Typical Class I and II HDAC inhibitors consist of a ZBG, a linker, and a cap group (FIG. 1) and are designed to mimic the endogenous ligand of the enzyme, acetyl lysine (FIG. 1). The cap group interacts with the outer rim of the HDAC catalytic groove, which varies among different HDAC isoforms, while the enzyme active site is relatively conserved. Compared to the linear HDAC inhibitors, romidepsin has a depsipeptide cap group and undergoes in situ activation by reduction of the disulfide bridge to release a thiol ZBG.

The most common HDAC inhibitors comprise hydroxamates, which are very potent, and chelate very tightly to the Zn²⁺ ion in a bidentate manner. However, hydroxamates have poor pharmacokinetic properties and their promiscuous metal-binding ability leads to HDAC-independent adverse effects, including cardiotoxicity, pulmonary embolism, thrombocytopenia with nausea, and diarrhea. In addition, the hydroxamates have the potential to undergo zinc-catalyzed Lossen-rearrangement to isocyanates, which are highly reactive electrophiles that can be targeted by numerous nucleophiles in the cells to cause a plethora of biological problems such as mutagenesis through reaction with nitrogen bases of DNA. Hydroxamates are also rapidly cleared metabolically, and they lack isoform selectivity. Hence, there is a need for alternative ZBGs. Even if the alternatives are less potent than hydroxamates, modulation of the cap group can be used to strike a balance between potency and selectivity, improving their pharmacologic properties and reducing possible drug side effects. Current alternatives to hydroxamates include molecules such as trifluoromethylketones (TFMKs), thiols, α-ketoamides, and HDAC 6-selective mercaptoacetamides. These inhibitors exhibit not only potent in vitro HDAC inhibitory activity, but also significant anti-tumor activity. Unfortunately, there is no established set of guidelines for designing ZBGs, and a direct correlation between zinc-binding ability and biological activity has not been fully established. The existence of some HDAC inhibitors without ZBGs adds to the complexity. Furthermore, the known alternatives have problems. For example, TFMKs are rapidly metabolized to inactive trifluoromethyl alcohols in vivo, having a half life of only about 0.5 hours. Furthermore, TFMKs have a very reactive electrophilic warhead that can form covalent bonds with many endogenous nucleophiles to produce off-target effects. These issues have prevented the further development of TFMKs as potential drug candidates.

There is a need in the art for new and improved HDAC inhibitors.

### SUMMARY

Provided is a composition comprising an HDAC inhibitor compound having a cap group, a linker, and a zinc binding group, wherein the zinc binding group comprises a trifluoromethylpyruvamide (TFMP) or a hydrate thereof.

Provided is a composition comprising Formula I:

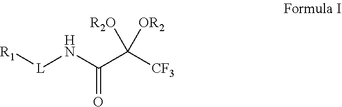

wherein R₁ is aryl, alkyl, heteroaryl, a hydrophobic moiety, a hydrophilic moiety, or a combination thereof, optionally substituted with one or more halogens, nitro groups, or amino groups; each R₂ is, independently, H or alkyl; and L is a linker comprising an alkyl chain, a fluorinated alkyl chain, an aryl group, an arylalkyl group, a carbonyl-containing chain, an amide-containing chain, or a heteroatom-containing chain, optionally substituted with one or more halogens, amino groups, or carboxylic acid groups. Also provided are salts, stereoisomers, racemates, solvates, hydrates, prodrugs, and polymorphs of Formula I.

In certain embodiments, R₁ comprises a benzyl, phenyl, pyridinyl, thiazolyl, or benzothiazolyl.

In certain embodiments, R₁ comprises a thiazolyl, benzyl, or benzothiazolyl; and L comprises a carbonyl-containing alkyl chain having from 1 to 10 carbons.

In certain embodiments, R₁ comprises a polyethylene glycol (PEG) chain.

In certain embodiments, L comprises an ether, a thioether, or an amide.

In certain embodiments, L is a carbonyl-containing alkyl chain having from 1 to 10 carbons.

In certain embodiments, the composition comprises compound 83:

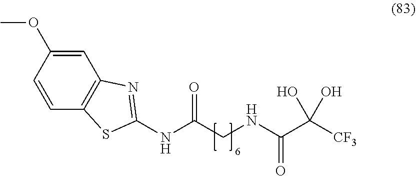

In certain embodiments, the composition comprises compound 84:

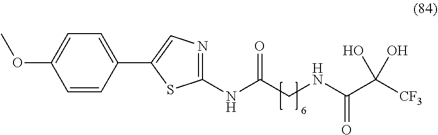

In certain embodiments, the composition comprises compound 85:

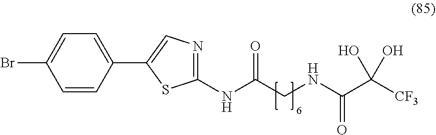

In certain embodiments, the composition comprises compound 86:

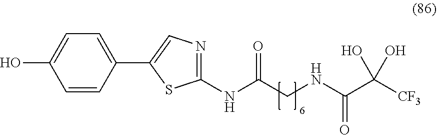

In certain embodiments, the composition comprises compound 61:

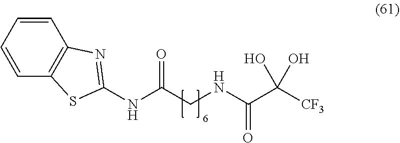

In certain embodiments, the composition comprises compound 70:

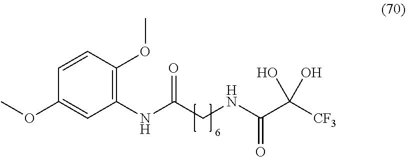

In certain embodiments, the composition comprises compound 64:

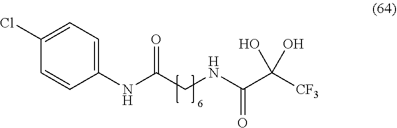

In certain embodiments, the composition comprises compound 65:

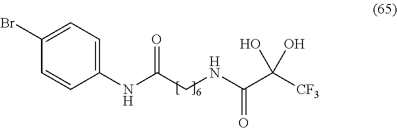

In certain embodiments, the composition comprises compound 69:

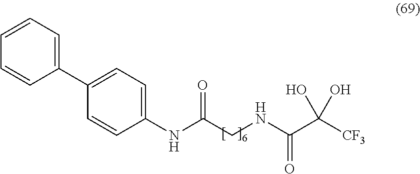

In certain embodiments, the composition comprises compound 58:

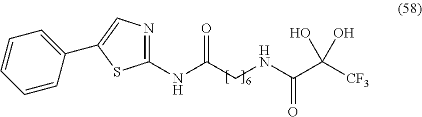

In certain embodiments, the composition comprises compound 55:

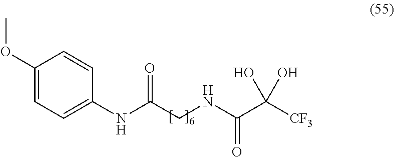

In certain embodiments, each R₂ is H.

Further provided is a pharmaceutical composition comprising an HDAC inhibitor compound described herein, and a pharmaceutically acceptable diluent, adjuvant, or carrier.

Further provided is a metal binding group comprising Formula II:

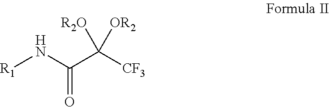

wherein R₁ is a lone pair of electrons, aryl, alkyl, heteroaryl, a hydrophobic moiety, a hydrophilic moiety, or a combination thereof, optionally substituted with one or more halogens, nitro groups, or amino groups; each R₂ is, independently, H or alkyl; and salts stereoisomers, racemates, hydrates, solvates, prodrugs, and polymorphs thereof. In certain embodiments, each R₂ is H.

Further provided is a method of inhibiting an HDAC protein, the method comprising contacting a cell with a composition comprising an HDAC inhibitor compound described herein and inhibiting an HDAC protein in the cell. In certain embodiments, the HDAC protein is HDAC8 and the composition comprises compound 83. In certain embodiments, the HDAC inhibitor compound is selected from the group consisting of compound 65, compound 61, compound 85, compound 69, compound 55, compound 86, compound 64, compound 83, compound 58, and compound 84.

Further provided is a method of inhibiting growth of cancer cells, the method comprising contacting cancer cells with an effective amount of a composition comprising an HDAC inhibitor compound described herein and inhibiting growth of the cancer cells.

In certain embodiments, the cancer is lung cancer, cervical cancer, breast cancer, colon cancer, fibrosarcoma, neuroblastoma, or osteosarcoma. In certain embodiments, the HDAC inhibitor compound is selected from the group consisting of compound 65, compound 61, compound 85, compound 69, compound 55, compound 86, compound 64, compound 83, compound 58, and compound 84.

Further provided is a method of synthesizing an HDAC inhibitor compound, the method comprising amide coupling an aminoalkanamide with a compound having a zinc binding group (ZBG) appended to a carboxylic acid group. In certain embodiments, the amide coupling is conducted with one or more coupling reagents selected from the group consisting of 1-ethyl-3-(3-dimethylaminopropyl)carbodiimide (EDCl), dimethylaminopyridine (DMAP) in dichloromethane, (1-[bis(dimethylamino)methylene]-1H-1,2,3-triazolo[4,5-b]pyridinium-3-oxide hexafluorophosphate (HATU), 1-hydroxybenzotriazole (HOBt), and N-methylmorpholine (NMM) in N,N-dimethylformamide (DMF).

Further provided is a method of synthesizing an HDAC inhibitor compound, the method comprising protecting an aminoalkanoic acid with a protecting group to obtain a protected aminoalkanoic acid; peptide coupling the protected aminoalkanoic acid with an aniline to obtain a protected amino anilide; and deprotecting and peptide coupling the protected amino anilide with trifluoropyruvic acid to obtain a trifluoropyruvamide. In certain embodiments, the protecting group is either tert-butyloxycarbonyl (Boc) or 2,2,2-trichloroethoxycarbonyl (Troc).

Further provided is a kit for making an HDAC inhibitor compound, the kit comprising a first container housing an aminoalkanoic acid; a second container housing an aniline; and a third container housing trifluoropyruvic acid.

Further provided is the use of trifluoromethylpyruvamide or a hydrate thereof as a metal binding group in an HDAC inhibitor.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1: HDAC inhibitors vorinostat (SAHA), belinostat (Beleodaq), panabinostat (Farydak), romidepsin (Istodax) in clinical use, and chidamide (Epidaza) in clinical trials. HDAC inhibitors are designed to mimic acetyl-lysine, as shown by a ZBG (red), linker (green), and a cap group (blue).

FIGS. 2A-2B: SAHA-like pharmacophore model used for HDAC inhibitor design (FIG. 2A), and some non-limiting example pyruvate-based ZBGs (FIG. 2B).

FIGS. 3A-3C: FIG. 3A shows a pyruvic amide (here compound 5c) typically shows a ¹³C NMR singlet at 197.2 ppm, which shows complete retention of the keto-form, despite the electron withdrawing ability of the adjacent amide group. FIG. 3B shows the ¹³C NMR of the TFMK 91, which shows the presence of a quartet at 191.5 ppm, despite the electron withdrawing ability of the trifluoromethyl group. FIG. 3C shows combining the amide and trifluoromethyl ketone groups resulted in a quartet at 94.3 ppm for compound 10c, which is characteristic of a hydrate.

FIG. 4: Adjusting the electrophilicity of carbonyl results in stabilized hydrates that can withstand carbonyl reductase reduction before reaching their targets inside cancer cells. Included is an alternate zinc-binding mode.

FIG. 5: Alignment of binding poses of each compound with SAHA inside the HDAC6 catalytic site.

FIGS. 6A-6E: FIG. 6A shows alignment of SAHA with TFMK and TFPA revealing similar binding modes on HDAC6, even though the linkers and cap groups of the TFPA are not as well aligned as the TFMK. FIG. 6B shows docking scores of analogs against several HDACs showing that TFPA, diamide (oxamide), and the pyruvamide to have similar values against several HDACs. FIG. 6C shows docking scores of compounds 14 and 10a-10d against several HDACs revealed 10b to be the best candidate. FIGS. 6D-6E show examination of the active site of HDAC6 with 10b and 10c, revealing that the inhibitors were bound to Tyr⁷⁴⁵, a residue that is essential for enzymatic activity.

FIG. 7: Scheme 1, depicting the synthesis of final products from aminoalkanilides 4a-4d. Conditions: a) EDCl/DMAP, dichloromethane, r.t, overnight. b) HATU/HOBt/NMM, DMF, 4h r.t. c) Oxalyl chloride/DMF (catalytic), DCM, r.t., overnight. d) NaOMe/MeOH, r.t., overnight.

FIG. 8: Schemes 2A-2C. Scheme 2A depicts the synthesis of aminoalkanilides 4a-4d. n=number of methylene groups. Scheme 2B depicts an alternative synthetic route using Boc-protected amino acids used to synthesize 14 and the SAR variants 53-87. n=number of methylene groups. Scheme 2C depicts the synthesis of compounds 51 and 52 from 50.

FIG. 9: Chain elongation study to find advantageous linker length (n=3-7).

FIG. 10: Cap groups selected for SAR studies of compound 10c.

FIG. 11: Cell growth inhibition assay of selected cell lines found compounds 84 and 85 to be more active than SAHA in almost all.

FIG. 12: Western blot of compound 84 and SAHA revealed increased levels of acetylated H3 and “pan”-acetyl-lysine in a dose dependent manner.

FIG. 13: Western blot analysis of PARP1 cleavage. HCT116 cells were treated with compounds 83, 84, and 85 at the indicated concentrations for 24 hr. Western blots were probed with an antibody that recognizes a cleaved form of PARP (Cell Signaling Technology 5626) as a measure of caspase dependent cell death.

FIGS. 14A-14B: FIG. 14A shows a plot of cell survival percentage against the concentration of 55 (shown as RB-5-55b) showing low IC₅₀ values against NCI-H522 and HCT116 cell lines, while RPE and WI38 cell lines displayed high IC₅₀ values. FIG. 14B shows a cell survival assay using compound 55 showing that HCT116 and H522 cancer cell lines have low survival rates (<20%) while normal cell lines WI38 and RPE have survival rates of >50%.

FIGS. 15A-15D: FIG. 15A shows the disappearance of the TFMK 91 302 M+1 peak in 15 minutes, followed by appearance of the TFMA 90 304 M+1 peak in 15 minutes (FIG. 15B). Highlighted next are the mass spectra of the TFMK 91 (FIG. 15C) and the TFMA 90 (FIG. 15D). Note: The LC and the mass spectrometer were operated using different computers. The sample run was started from the autosampler using a method in one computer that causes the compound to flow through the column into the mass spectrometer, following which the mass spectra were recorded using a different program in another computer. This lack of synchronicity caused a slight difference in the retention time for the same compound in different runs.

FIGS. 16A-16C: LCMS studies of the S9 fraction assay of compound 60 show no disappearance of the 370 M+1 peak over time. LCMS studies revealed that its 354 M+1 peak did not form over time. Also included are the M+1 peaks of compounds 60 and 92 (FIGS. 16B, 16C).

FIGS. 17A-17C: FIG. 17A shows a trifluoromethylketone (TFMKs) acting as an alternative ZBG. FIG. 17B shows TFMKs having a very reactive electrophilic wardhead that can form covalent bonds with many endogenous nucleophiles to produce off-target effects. FIG. 17C shows a general scheme showing trifluoromethylpyruvamides (TFPAs) binding Zn²⁺ ions as TFMK surrogates.

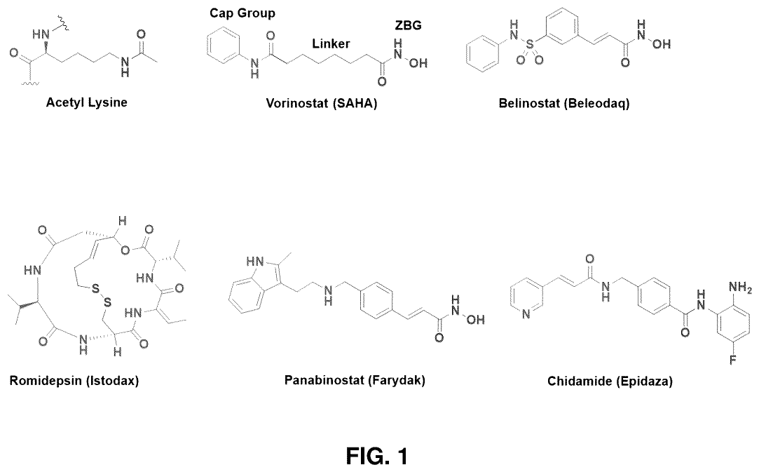

### DETAILED DESCRIPTION

Throughout this disclosure, various publications, patents, and published patent specifications are referenced by an identifying citation. The disclosures of these publications, patents, and published patent specifications are hereby incorporated by reference into the present disclosure in their entirety to more fully describe the state of the art to which this invention pertains.

To overcome limitations with known HDAC inhibitors, trifluoropyruvamides (TFPAs) have been developed as ZBGs that act as TFMK surrogates. The trifluoromethylpyruvamide (TFMP) ZBG was developed by incorporating another electron withdrawing carbonyl group into the TFMK group. This stabilizes the hydrate and makes it stable to metabolic reduction. The presence of an additional electron withdrawing group next to the ketone carbonyl group makes the hydrate form of the ketone more stable, thus preventing its metabolic reduction to alcohol in vivo. Accordingly, TFPA is more metabolically stable than TFMK. In addition, this structural modification reduces the potential of the TFMK group to act as a covalent warhead to eliminate off-target effects. As shown in the examples herein, additional structural changes in the cap group of the inhibitors gave analogues with IC₅₀ values ranging from upper nanomolar to low micromolar in the cytotoxicity assay, and they were more selective for cancer cells over normal cells. Some of the most active analogues inhibited HDAC enzymes with low nanomolar IC₅₀ values and were found to be more selective for HDAC8 over other isoforms. Furthermore, TFPAs cannot act as pan assay interference (PAINS) molecules, which are frequent false positives in high-throughput screening assays. These molecules provide a new class of HDAC inhibitors with a metabolically stable metal-binding group that can also be used to develop selective HDAC inhibitors by further structural modification.

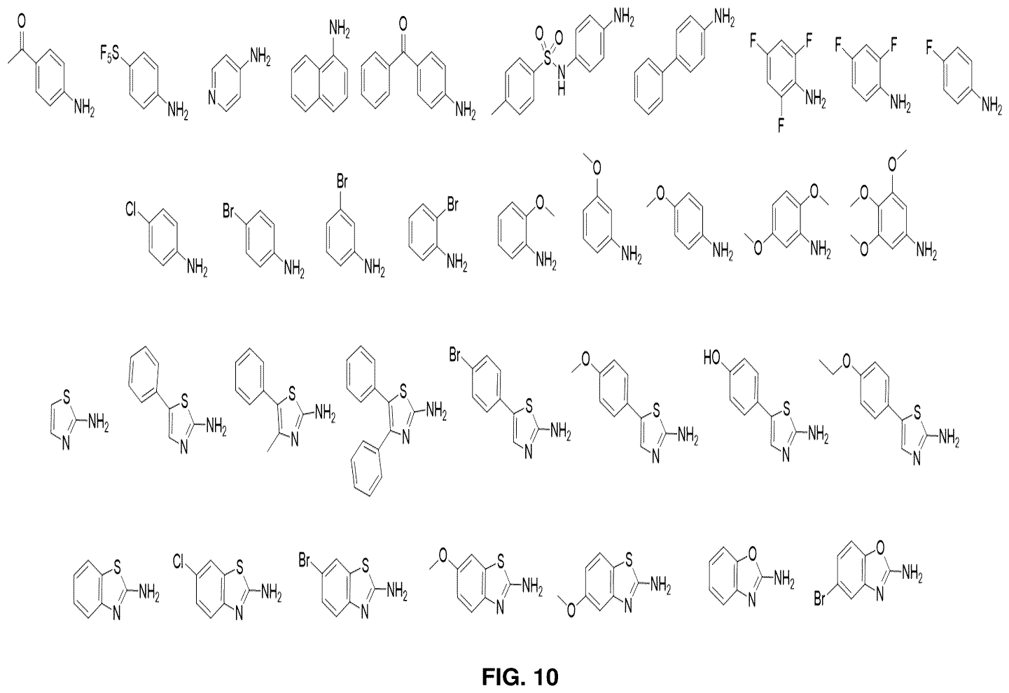

In general, the HDAC inhibitor compounds herein have the structural formula of Formula I:

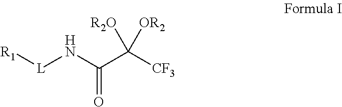

where R₁ is a cap group, each R₂ is independently H or alkyl, and L is a linker. The cap group is selected from a variety of possible chemical moieties that are capable of enhancing the potency, selectivity, and pharmacokinetic properties of the HDAC inhibitor compound. The cap group can be an aryl, alkyl, or heteroaryl moiety, or a combination thereof, such as benzyl, phenyl, pyridinyl, thiazolyl, or benzothiazolyl. Alternatively, the cap group can be a hydrophobic or hydrophilic moiety, such as an alkyl chain or a polyethylene glycol (PEG) chain. The cap group may modulate the physicochemical properties of the HDAC inhibitor compound, such as lipophilicity and water solubility. In some embodiments, R₁ is one of the cap groups depicted in FIG. 10. In some embodiments, the cap group may be substituted with one or more functional groups, such as halogens, nitro groups, or amino groups, which can modulate the physicochemical properties of the HDAC inhibitor compound. For example, the presence of a halogen substituent on the cap group can increase lipophilicity and enhance cell permeability, while the presence of an amino group can increase water solubility and improve pharmacokinetic properties.

As noted, R₂ can be either H or an alkyl group. Suitable alkyl groups for R₂ include, but are not limited to, methyl, ethyl, propyl, butyl, secbutyl, isobutyl, tertbutyl, pentyl, hexyl, heptyl, octyl, nonyl, and decyl.

The linker of Formula I connects the cap group to the metal binding group. The linker may be a simple alkyl chain, a fluorinated alkyl chain, an aryl group, an arylalkyl group, a carbonyl-containing chain, an amide-containing chain, or a heteroatom-containing chain, such as an ether, thioether, or amide. The linker can also be substituted with functional groups, such as halogens, amino groups, or carboxylic acid groups, which can modulate the physicochemical properties of the HDAC inhibitor compound. In some embodiments, the linker is a carbonyl-containing alkyl chain (i.e., an acyl group) having from 1 to 10 carbons. In some embodiments, the linker includes an ether, a thioether, or an amide.

In some embodiments, the cap group R₁ includes one of a thiazolyl, a benzyl, or a benzothiazolyl while the linker L includes a carbonyl-containing alkyl chain having from 1 to 10 carbons.

Non-limiting example HDAC inhibitor compounds of Formula I include each of the following compounds 83, 84, 85, 86, 61, 70, 64, 65, 69, 58, and 55:

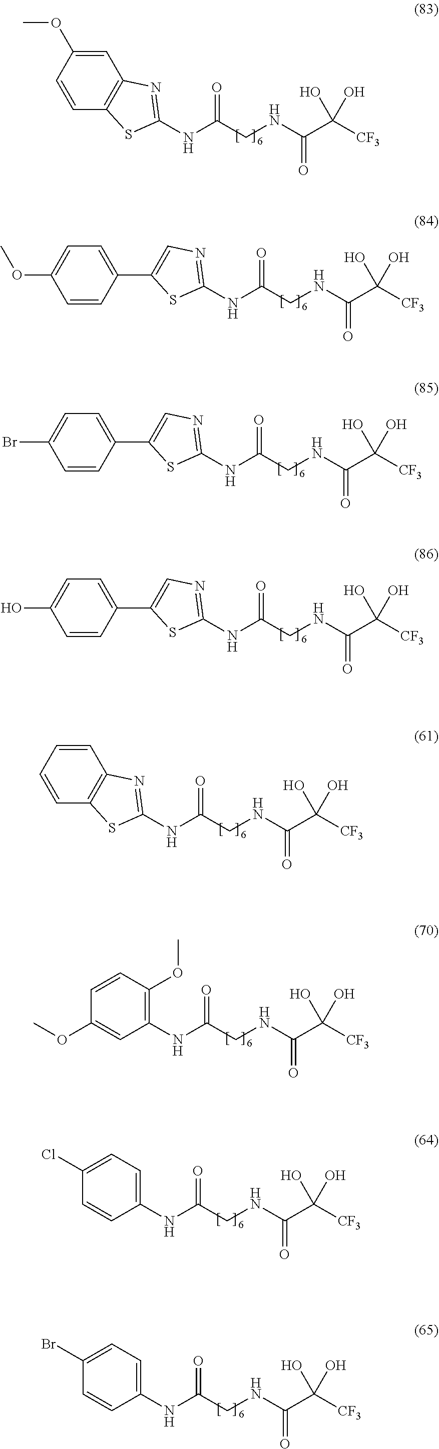

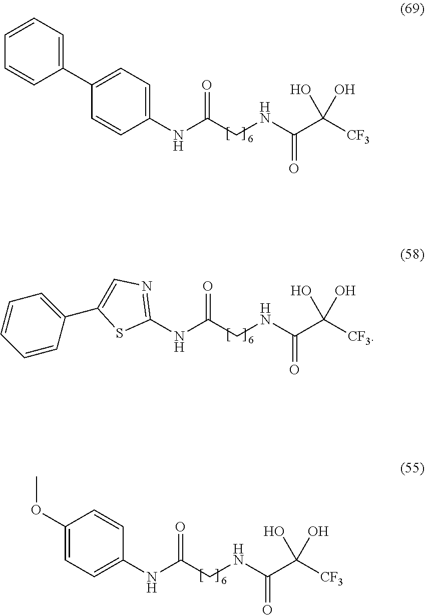

As shown in the examples herein, compounds 83, 84, 85, 86, 61, 70, 64, 65, 69, 58, and 55 are effective at inhibiting HDAC activity and inhibiting cell growth of various cancer cells. Furthermore, these compounds are metabolically stable.

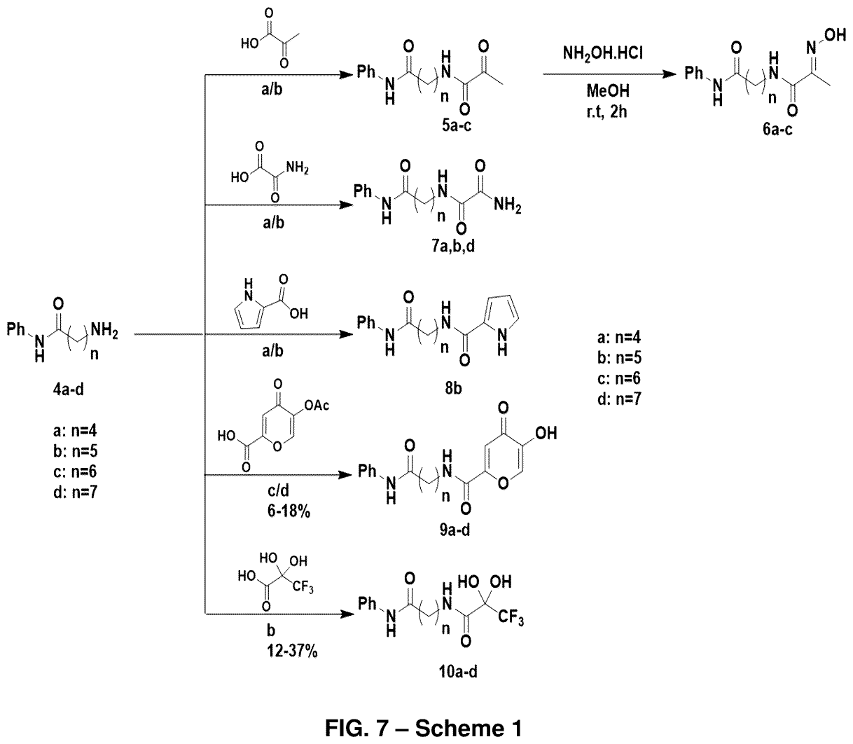

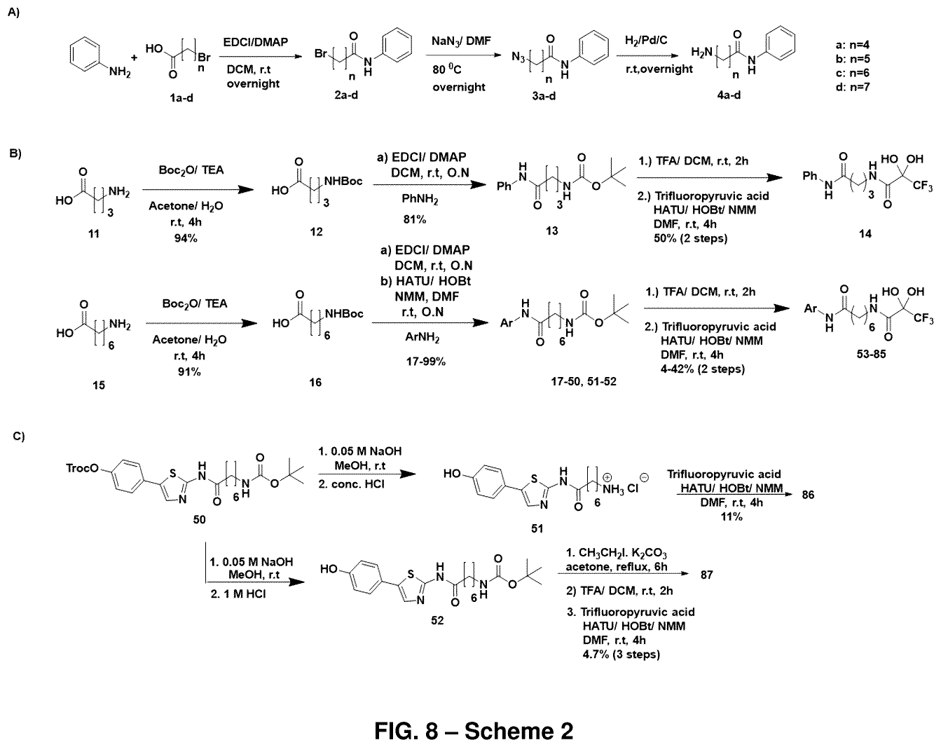

The HDAC inhibitor compounds of Formula I can be synthesized according to the schemes depicted in FIGS. 7-8. As shown in FIG. 7, a method for synthesizing an HDAC inhibitor compound may involve amide coupling of an aminoalkanilide with a compound that includes a ZBG appended to a carboxylic acid group. As non-limiting example coupling reagents, 1-ethyl-3-(3-dimethylaminopropyl)carbodiimide (EDCl), dimethylaminopyridine (DMAP) in dichloromethane, (1-[bis(dimethylamino)methylene]-1H-1,2,3-triazolo[4,5-b]pyridinium-3-oxide hexafluorophosphate (HATU), 1-hydroxybenzotriazole (HOBt), and/or N-methylmorpholine (NMM) in N,N-dimethylformamide (DMF) can be used as coupling reagents to accomplish the amide coupling. Using HATU, HOBt, and NMM in the presence of molecular sieves under anhydrous conditions at room temperature for four hours can successfully synthesize trifluoropyruvamides using this approach.

As seen in FIG. 8, a method for synthesizing an HDAC inhibitor compound may begin with a commonly available aminoalkanoic acid such as γ-aminobutyric acid (GABA), and may involve protecting the aminoalkanoic acid with a protecting group to obtain a protected aminoalkanoic acid, peptide coupling the protected aminobutyric acid with an aniline to obtain a protected amino anilide, and deprotecting and peptide coupling the protected amino anilide with trifluoropyruvic acid to obtain a trifluoropyruvamide. In some embodiments, the protecting group is either tert-butyloxycarbonyl (Boc) or 2,2,2-trichloroethoxycarbonyl (Troc). In some embodiments, the method further includes ethylating a deprotected amino anilide prior to the peptide coupling with the trifluoropyruvic acid. Various aromatic amines, such as 2-aminothiazoles, can be used as cap groups.

Also provided are metal binding groups having the general structural formula of Formula II:

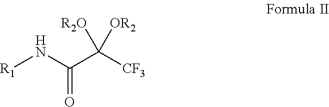

where R₁ is lone pair of electrons, aryl, alkyl, or heteroaryl, a hydrophobic moiety, or a hydrophilic moiety, optionally substituted with one or more halogens, nitro groups, or amino groups; and each R₂ is, independently, H or alkyl. Suitable alkyl groups for R₂ include, but are not limited to, methyl, ethyl, propyl, butyl, secbutyl, isobutyl, tertbutyl, pentyl, hexyl, heptyl, octyl, nonyl, and decyl. The metal binding groups are useful not only in HDAC inhibitor compounds, but also in any other drug molecules which include a metal binding group such as, but not limited to, metalloproteinase inhibitors, HIV-1 integrase enzyme targeting drugs, protease inhibitors, and kinase inhibitors.

The trifluoromethylketone group previously developed as an alternative, and effective, metal binding group, undergoes rapid metabolic reduction resulting in loss of HDAC inhibitory activity. TFPA is effective as a ZBG for HDAC inhibitors, and is not susceptible to metabolic reduction, does not act as PAINS, is not susceptible to reacting with endogenous reactive nucleophiles, is less likely to cause off-target effects than TFMKs, and results in potent HDAC inhibitors. Numerous effective HDAC inhibitors, useful for treating certain cancers, have been synthesized with a TFPA ZBG.

Pharmaceutical compositions of the present disclosure may comprise an effective amount of an HDAC inhibitor compound described herein, and/or additional agents, dissolved or dispersed in a pharmaceutically acceptable carrier. The preparation of a pharmaceutical composition will be known to those of skill in the art in light of the present disclosure, as exemplified by Remington's Pharmaceutical Sciences, 2003, incorporated herein by reference. Moreover, for animal (e.g., human) administration, it is understood that preparations should meet sterility, pyrogenicity, general safety, and purity standards as required by FDA Office of Biological Standards.

A composition disclosed herein may comprise different types of carriers depending on whether it is to be administered in solid, liquid, or aerosol form, and whether it needs to be sterile for such routes of administration as injection. Compositions disclosed herein can be administered intravenously, intradermally, transdermally, intrathecally, intraarterially, intraperitoneally, intranasally, intravaginally, intrarectally, intraosseously, periprosthetically, topically, intramuscularly, subcutaneously, mucosally, intraosseosly, periprosthetically, in utero, orally, topically, locally, via inhalation (e.g., aerosol inhalation), by injection, by infusion, by continuous infusion, by localized perfusion bathing target cells directly, via a catheter, via a lavage, in cremes, in lipid compositions (e.g., liposomes), or by other method or any combination of the foregoing as would be known to one of ordinary skill in the art (see, for example, Remington's Pharmaceutical Sciences, 2003, incorporated herein by reference).

The actual dosage amount of a composition disclosed herein administered to an animal or human patient can be determined by physical and physiological factors such as body weight, severity of condition, the type of disease being treated, previous or concurrent therapeutic interventions, idiopathy of the patient, and on the route of administration. Depending upon the dosage and the route of administration, the number of administrations of a preferred dosage and/or an effective amount may vary according to the response of the subject. The practitioner responsible for administration will, in any event, determine the concentration of active ingredient(s) in a composition and appropriate dose(s) for the individual subject.

In certain embodiments, a composition herein and/or additional agent is formulated to be administered via an alimentary route. Alimentary routes include all possible routes of administration in which the composition is in direct contact with the alimentary tract. Specifically, the pharmaceutical compositions disclosed herein may be administered orally, buccally, rectally, or sublingually. As such, these compositions may be formulated with an inert diluent or with an assimilable edible carrier, or they may be enclosed in hard- or soft-shell gelatin capsules, they may be compressed into tablets, or they may be incorporated directly with the food of the diet.

In further embodiments, a composition described herein may be administered via a parenteral route. As used herein, the term “parenteral” includes routes that bypass the alimentary tract. Specifically, the pharmaceutical compositions disclosed herein may be administered, for example but not limited to, intravenously, intradermally, intramuscularly, intraarterially, intrathecally, subcutaneous, or intraperitoneally (U.S. Pat. Nos. 6,753,514, 6,613,308, 5,466,468, 5,543,158; 5,641,515, and 5,399,363 are each specifically incorporated herein by reference in their entirety).

Solutions of the compositions disclosed may be prepared in water suitably mixed with a surfactant, such as hydroxypropylcellulose. Dispersions may also be prepared in glycerol, liquid polyethylene glycols and mixtures thereof, and in oils. Under ordinary conditions of storage and use, these preparations may contain a preservative to prevent the growth of microorganisms. The pharmaceutical forms suitable for injectable use include sterile aqueous solutions or dispersions and sterile powders for the extemporaneous preparation of sterile injectable solutions or dispersions (U.S. Pat. No. 5,466,468, specifically incorporated herein by reference in its entirety). In some cases, the form should be sterile and should be fluid to the extent that easy injectability exists. It should be stable under the conditions of manufacture and storage and should be preserved against the contaminating action of microorganisms, such as bacteria and fungi. The carrier can be a solvent or dispersion medium containing, for example, water, ethanol, polyol (i.e., glycerol, propylene glycol, liquid polyethylene glycol, and the like), suitable mixtures thereof, and/or vegetable oils. Proper fluidity may be maintained, for example, by the use of a coating, such as lecithin, by the maintenance of the required particle size in the case of dispersion, and/or by the use of surfactants. The prevention of the action of microorganisms can be brought about by various antibacterial and antifungal agents, such as, but not limited to, parabens, chlorobutanol, phenol, sorbic acid, thimerosal, and the like. In some cases, it may be preferable to include isotonic agents, for example, sugars or sodium chloride. Prolonged absorption of the injectable compositions can be brought about by the use in the compositions of agents delaying absorption such as, for example, aluminum monostearate or gelatin.

For parenteral administration in an aqueous solution, for example, the solution should be suitably buffered if necessary and the liquid diluent first rendered isotonic with sufficient saline or glucose. These particular aqueous solutions are especially suitable for intravenous, intramuscular, subcutaneous, and intraperitoneal administration. In this connection, sterile aqueous media that can be employed will be known to those of skill in the art in light of the present disclosure. For example, one dosage may be dissolved in 1 mL of isotonic NaCl solution and either added to 1000 mL of hypodermoclysis fluid or injected at the proposed site of infusion, (see for example, “Remington's Pharmaceutical Sciences” 15th Edition, pages 1035-1038 and 1570-1580). Some variation in dosage will necessarily occur depending on the condition of the subject being treated. The person responsible for administration will, in any event, determine the appropriate dose for the individual subject.

Sterile injectable solutions are prepared by incorporating the compositions in the required amount in the appropriate solvent with various other ingredients enumerated above, as required, followed by filtered sterilization. Generally, dispersions are prepared by incorporating the various sterilized compositions into a sterile vehicle which contains the basic dispersion medium and the required other ingredients from those enumerated above.

In other embodiments, the compositions may be formulated for administration via various miscellaneous routes, for example, topical (i.e., transdermal) administration, mucosal administration (intranasal, vaginal, etc.), and/or via inhalation.

Pharmaceutical compositions for topical administration may include the compositions formulated for a medicated application such as an ointment, paste, cream, or powder. Ointments include all oleaginous, adsorption, emulsion, and water-soluble based compositions for topical application, while creams and lotions are those compositions that include an emulsion base only. Topically administered medications may contain a penetration enhancer to facilitate adsorption of the active ingredients through the skin. Suitable penetration enhancers include glycerin, alcohols, alkyl methyl sulfoxides, pyrrolidones, and luarocapram. Possible bases for compositions for topical application include polyethylene glycol, lanolin, cold cream, and petrolatum, as well as any other suitable absorption, emulsion, or water-soluble ointment base. Topical preparations may also include emulsifiers, gelling agents, and antimicrobial preservatives as necessary to preserve the composition and provide for a homogenous mixture. Transdermal administration of the compositions may also comprise the use of a “patch.” For example, the patch may supply one or more compositions at a predetermined rate and in a continuous manner over a fixed period of time.

In certain embodiments, the compositions may be delivered by eye drops, intranasal sprays, inhalation, and/or other aerosol delivery vehicles. Methods for delivering compositions directly to the lungs via nasal aerosol sprays has been described in U.S. Pat. Nos. 5,756,353 and 5,804,212 (each specifically incorporated herein by reference in their entirety). Likewise, the delivery of drugs using intranasal microparticle resins (Takenaga et al., 1998) and lysophosphatidyl-glycerol compounds (U.S. Pat. No. 5,725,871, specifically incorporated herein by reference in its entirety) are also well-known in the pharmaceutical arts and could be employed to deliver the compositions described herein. Likewise, transmucosal drug delivery in the form of a polytetrafluoroetheylene support matrix is described in U.S. Pat. No. 5,780,045 (specifically incorporated herein by reference in its entirety), and could be employed to deliver the compositions described herein.

It is further envisioned the compositions disclosed herein may be delivered via an aerosol. The term aerosol refers to a colloidal system of finely divided solid or liquid particles dispersed in a liquefied or pressurized gas propellant. The typical aerosol for inhalation consists of a suspension of active ingredients in liquid propellant or a mixture of liquid propellant and a suitable solvent. Suitable propellants include hydrocarbons and hydrocarbon ethers. Suitable containers will vary according to the pressure requirements of the propellant. Administration of the aerosol will vary according to subject's age, weight, and the severity and response of the symptoms.

In particular embodiments, the compounds and compositions described herein are useful for treating cancers or inhibiting growth of cancer cells. As described herein, the compounds and compositions herein can be used in combination therapies. That is, the compounds and compositions can be administered concurrently with, prior to, or subsequent to one or more other desired therapeutic or medical procedures or drugs. The particular combination of therapies and procedures in the combination regimen will take into account compatibility of the therapies and/or procedures and the desired therapeutic effect to be achieved. Combination therapies include sequential, simultaneous, and separate administration of the active compound in a way that the therapeutic effects of the first administered procedure or drug is not entirely disappeared when the subsequent procedure or drug is administered.

In some embodiments, the HDAC inhibitor compound is part of a combination therapy with a chemotherapeutic agent. Suitable chemotherapeutic agents include, but are not limited to: taxane compounds, such as paclitaxel; platinum coordination compounds; topoisomerase I inhibitors, such as camptothecin compounds; topoisomerase II inhibitors, such as anti-tumor podophyllotoxin derivatives; anti-tumor vinca alkaloids; anti-tumor nucleoside derivatives; alkylating agents; anti-tumor anthracycline derivatives; HER2 antibodies; estrogen receptor antagonists or selective estrogen receptor modulators; aromatase inhibitors; differentiating agents, such as retinoids, and retinoic acid metabolism blocking agents (RAMBA); DNA methyl transferase inhibitors; kinase inhibitors; farnesyltransferase inhibitors; HDAC inhibitors, or other inhibitors of the ubiquitin-proteasome pathway; alkyl sulfonates such as busulfan, improsulfan and piposulfan; aziridines such as benzodopa, carboquone, meturedopa, and uredopa; ethylenimines and methylamelamines including altretamine, triethylenemelamine, triethylenephosphoramide, triethylenethiophosphoramide, and trimethylomelamine; acetogenins; camptothecins, such as the synthetic analog topotecan; cryptophycins; nitrogen mustards, such as chlorambucil; nitrosoureas; bisphosphonates; mitomycins; epothilones; maytansinoids; trichothecenes; retinoids, such as retinoic acid; pharmaceutically acceptable salts, acids and derivatives of any of the above; and combinations thereof. Non-limiting examples of specific chemotherapeutic agents include erlotinib (TARCEVA®, Genentech/OSI Pharm.), docetaxel (TAXOTERE®, Sanofi-Aventis), 5-FU (fluorouracil, 5-fluorouracil, CAS No. 51-21-8), gemcitabine (GEMZAR®, Lilly), PD-0325901 (CAS No. 391210-10-9, Pfizer), cisplatin (cis-diamine, dichloroplatinum(II), CAS No. 15663-27-1), carboplatin (CAS No. 41575-94-4), paclitaxel (TAXOL®, Bristol-Myers Squibb Oncology), temozolomide (4-methyl-5-oxo-2,3,4,6,8-pentazabicyclo[4.3.0]nona-2,7,9-triene-9-carboxamide, CAS No. 85622-93-1, TEMODAR®, TEMODAL®, Schering Plough), tamoxifen ((Z)-2-[4-(1,2-diphenylbut-1-enyl)phenoxy]-N,N-dimethyl-ethanamine, NOLVADEX®, ISTUBAL®, VALODEX®), doxorubicin (ADRIAMYCIN®), Akti-1/2, HPPD, rapamycin, lapatinib (TYKERB®, Glaxo SmithKline), oxaliplatin (ELOXATIN®, Sanofi), bortezomib (VELCADE®, Millennium Pharm.), sutent (SUNITINIB®, SU11248, Pfizer), letrozole (FEMARA®, Novartis), imatinib mesylate (GLEEVEC®, Novartis), XL-518 (MEK inhibitor, Exelixis, WO 2007/044515), ARRY-886 (MEK inhibitor, AZD6244, Array BioPharma, Astra Zeneca), SF-1126 (PI3K inhibitor, Semafore Pharmaceuticals), BEZ-235 (PI3K inhibitor, Novartis), XL-147 (PI3K inhibitor, Exelixis), ABT-869 (multi-targeted inhibitor of VEGF and PDGF family receptor tyrosine kinases, Abbott Laboratories and Genentech), ABT-263 (Bcl-2/Bcl-xL inhibitor, Abbott Laboratories and Genentech), PTK787/ZK 222584 (Novartis), fulvestrant (FASLODEX®, AstraZeneca), leucovorin (folinic acid), lonafamib (SARASAR™, SCH 66336, Schering Plough), sorafenib (NEXAVAR®, BAY43-9006, Bayer Labs), gefitinib (IRESSA®, AstraZeneca), irinotecan (CAMPTOSAR®, CPT-11, Pfizer), tipifamib (ZARNESTRA™, Johnson & Johnson), capecitabine (XELODA(®, Roche), ABRAXANE™ (Cremophor-free), albumin-engineered nanoparticle formulations of paclitaxel (American Pharmaceutical Partners, Schaumberg, Ill.), vandetanib (rINN, ZD6474, ZACTIMA®, AstraZeneca), chloranmbucil, AG1478, AG1571 (SU 5271; Sugen), temsirolimus (TORISEL®, Wyeth), pazopanib (GlaxoSmithKline), canfosfamide (TELCYTA®, Telik), thioTepa and cyclosphosphamide (CYTOXAN®, NEOSAR®), bullatacin, bullatacinone, bryostatin, callystatin, CC-1065 (including its adozelesin, carzelesin and bizelesin synthetic analogs), cryptophycin 1, cryptophycin 8, dolastatin, duocarmycin (including the synthetic analogs, KW-2189 and CB1-TM1), leutherobin, pancratistatin, sarcodictyin, spongistatin, chlomaphazine, chlorophosphamide, estramustine, ifosfamide, mechlorethamine, mechlorethamine oxide hydrochloride, melphalan, novembichin, phenesterine, prednimustine, trofosfamide, uracil mustard, carmustine, chlorozotocin, fotemustine, lomustine, nimustine, ranimnustine, clodronate, esperamicin, neocarzinostatin chromophore and related chromoprotein enediyne antibiotic chromophores, aclacinomysins, actinomycin, authramycin, azaserine, bleomycins, cactinomycin, carabicin, caminomycin, carzinophilin, chromomycinis, dactinomycin, daunorubicin, detorubicin, 6-diazo-5-oxo-L-norleucine, morpholino-doxorubicin, cyanomorpholino-doxorubicin, 2-pyrrolino-doxorubicin and deoxydoxorubicin), epirubicin, esorubicin, idarubicin, marcellomycin, mitomycin C, mycophenolic acid, nogalamycin, olivomycins, peplomycin, porfiromycin, puromycin, quelamycin, rodorubicin, streptonigrin, streptozocin, tubercidin, ubenimex, zinostatin, zorubicin, methotrexate, 5-fluorouracil (5-FU), denopterin, methotrexate, pteropterin, trimetrexate, fludarabine, 6-mercaptopurine, thiamiprine, thioguanine, ancitabine, azacitidine, 6-azauridine, carmofur, cytarabine, dideoxyuridine, doxifluridine, enocitabine, floxuridine, calusterone, dromostanolone propionate, epitiostanol, mepitiostane, testolactone, aminoglutethimide, mitotane, trilostane, frolinic acid, aceglatone, aldophosphamide glycoside, aminolevulinic acid, eniluracil, amsacrine, bestrabucil, bisantrene, edatraxate, defofamine, demecolcine, diaziquone, elformithine, elliptinium acetate, etoglucid, gallium nitrate, hydroxyurea, lentinan, lonidainine, maytansine, ansamitocins, mitoguazone, mitoxantrone, mopidanmol, nitraerine, pentostatin, phenamet, pirarubicin, losoxantrone, podophyllinic acid, 2-ethylhydrazide, procarbazine, PSK® polysaccharide complex (JHS Natural Products, Eugene, Oreg.), razoxane, rhizoxin, sizofuran, spirogermanium, tenuazonic acid, triaziquone, 2,2′,2″-trichlorotriethylamine, T-2 toxin, verracurin A, roridin A, anguidine, urethane, vindesine, dacarbazine, mannomustine, mitobronitol, mitolactol, pipobroman, gacytosine, arabinoside (“Ara-C”), cyclophosphamide, thioTepa, 6-thioguanine, mercaptopurine, vinblastine, etoposide (VP-16), ifosfamide, mitoxantrone, vincristine, vinorelbine (NAVELBINE®), novantrone, teniposide, edatrexate, daunomycin, aminopterin, ibandronate, CPT-11, topoisomerase inhibitor RFS 2000, and difluoromethylomithine (DMFO), paclitaxel, 5-fluorouracil, abraxane (paclitaxel albumin-stabilized nanoparticle formulation), afinitor (everolimus), erlotinib hydrochloride, everolimus, gemcitabine hydrochloride, oxaliplatin (eloxatin), capecitabine (xeloda), cisplatin, irinotecan (camptosar), colinic acid (leucovorin), folfox (folinic acid, 5-fluorouracil, and oxaliplatin), folfirinox (folinic acid, 5-fluorouracil, irinotecan, and oxaliplatin), nab-paclitaxel with gemcitabine, metformin, digoxin, and simvastatin.

In some embodiments, the HDAC inhibitor compound is part of a combination therapy with an immunotherapeutic agents. Non-limiting examples of immunotherapeutic agents include nivolumab, pembrolizumab, rituximab, durvalumab, cemiplimab, and combinations thereof.

In some embodiments, the HDAC inhibitor compound is part of a combination therapy with a hormonal therapeutic agent. Non-limiting examples of hormonal therapeutic agents include anastrozole, exemestane, letrozole, tamoxifen, raloxifene, fulvestrant, toremifene, gosrelin, leuprolide, triptorelin, apalutamide, enzalutamide, darolutamide, bicalutamide, flutamide, nilutamide, abiraterone, ketoconazole, degarelix, medroxyprogesterone acetate, megestrol acetate, mitotane, and combinations thereof.

The compositions and HDAC inhibitor compounds described herein may also be made available via a kit containing one or more key components. A non-limiting example of such a kit for making an HDAC inhibitor compound comprises an aminoalkanoic acid, an aniline, and trifluoropyruvic acid in separate containers, where the containers may or may not be present in a combined configuration. Many other kits, such as kits which further include one or more coupling reagents, are possible and encompassed within the scope of the present disclosure. The kits may further include instructions for using the components of the kit to prepare HDAC inhibitor compounds. The instructions may be recorded on a suitable recording medium. For example, the instructions may be present in the kits as a package insert or in the labeling of the container of the kit or components thereof. In other embodiments, the instructions are present as an electronic storage data file present on a suitable computer readable storage medium, such as a flash drive. In other embodiments, the actual instructions are not present in the kit, but means for obtaining the instructions from a remote source, such as via the internet, are provided. An example of this embodiment is a kit that includes a web address where the instructions can be viewed and/or from which the instructions can be downloaded. As with the instructions, this means for obtaining the instructions is recorded on a suitable substrate.

### Examples

These examples describe the development of HDAC inhibitors with ZBGs of moderate Zn²⁺ binding ability and higher selectivity. Selection of appropriate metal-binding groups was based on the belief that bidentate chelation leading to five-membered metallocycle intermediates/transition states is mostly preferred due to lack of strain. Among the alternates to SAHA, trifluoromethyl ketones (TFMKs) have been shown to be potent HDAC inhibitors, but they suffer from rapid reductive metabolism of the ketone carbonyl group in vivo, limiting their use as anticancer agents. To overcome this metabolic impediment, trifluoropyruvamides (TFPAs) were designed and synthesized as metabolically more stable variants of TFMK HDAC inhibitors.

### Results and Discussion

### Design and Molecular Modeling

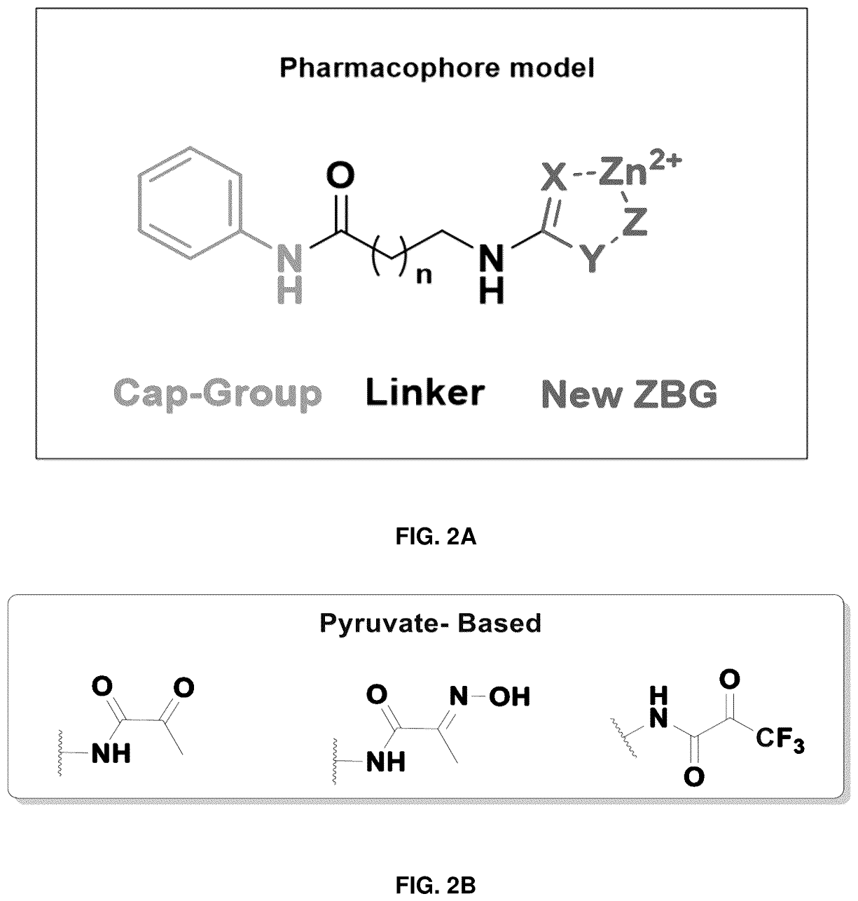

Computational analysis has revealed several possible bidentate ZBGs (FIG. 2) that can be explored to design effective HDAC inhibitors. For investigation of the efficacy of these ZBGs, HDAC inhibitors were designed using a “SAHA-like” model (FIG. 2) and their antiproliferative activity was compared with that of SAHA. This model utilized anilides as the cap group and an alkyl linker. To determine the optimal length of the linker, alkyl chains of different lengths were used. During preliminary biological screening (Table 1), it was observed that among the several designed ZBGs, only the TFPA 10b demonstrated antiproliferative activity. TFPAs are structural variants of TMFKs, which were originally developed around the same time as SAHA. These molecules bind to HDACs using a four-membered gem-diol-based metallocycle transition state using their hydrate form. However, TFMKs, especially the linear analogs, have exhibited poor half-lives of under 15 minutes as they are prone to reductive metabolism by carbonyl reductases (CBRs) in vivo. Since then, several variants have been synthesized, but without any reported improvements of the stability of the ZBG. TFPAs resulted from the incorporation of an additional electron-withdrawing carbonyl functional group to increase the metabolic stability of the trifluoromethyl ketone via stabilization of its hydrate.

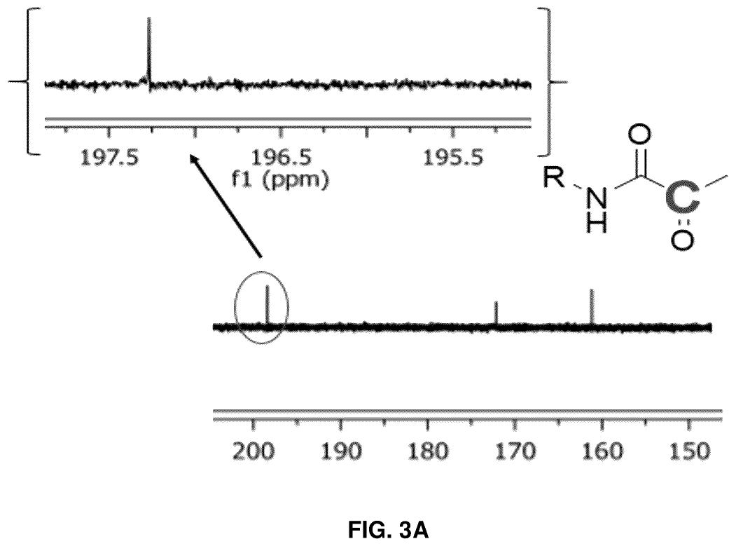

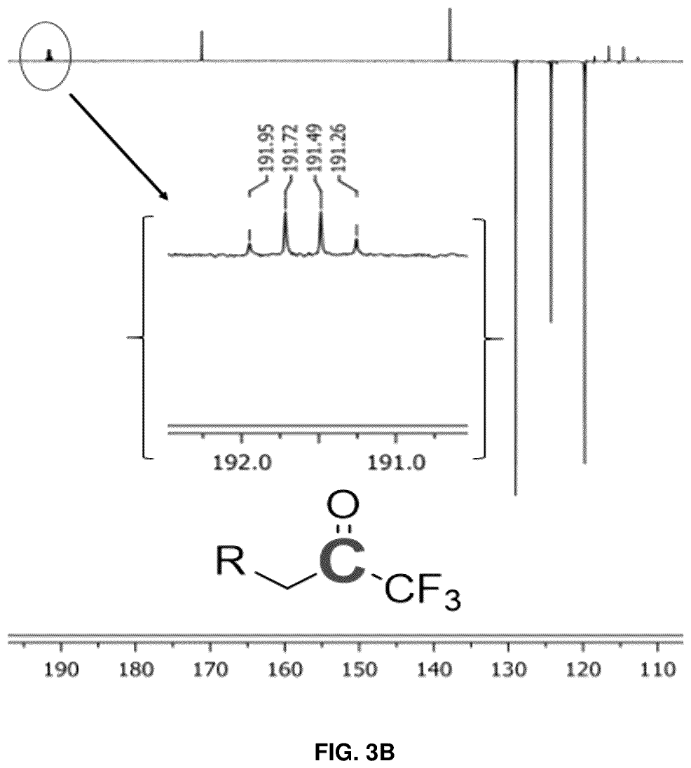

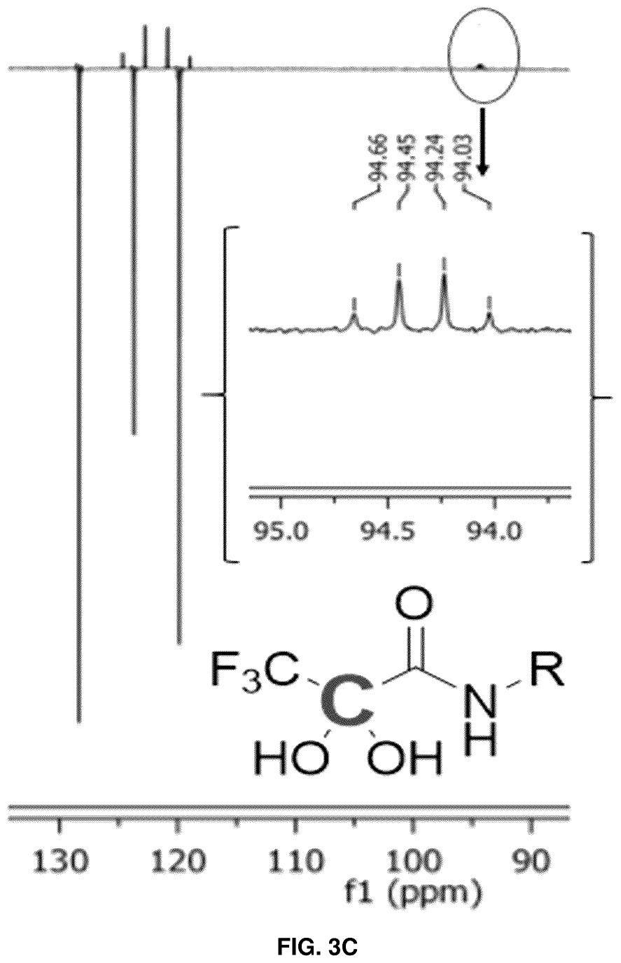

Without wishing to be bound by theory, it is believed that HDAC-TFMK interactions via the hydrate formed inside the active site are induced due to enhancement of electrophilicity of the carbonyl group by zinc-chelation, facilitating nucleophilic addition of a water molecule. The keto-form is predominant elsewhere as indicted by the appearance of the carbonyl carbon as a quartet at 191.5 ppm in the ¹³C NMR spectrum of the TFMK analogue (FIG. 3B). It is susceptible to reductive metabolism by carbonyl reductases (CBRs) in the human body. The incorporation of an additional electron withdrawing group buttresses the effect of the trifluoromethyl group and stabilizes the hydrate form, making it resistant to reductive metabolism. To confirm this, two different approaches were used. The first approach led to a perfluoro aryl-TFMK (compound 89, Table 1), which was inactive, presumably due to the steric nature of the aromatic ring. The second design that led to TFPAs incorporated an amide carbonyl as an extra electron withdrawing group to TFMK, while retaining its small size. This modification does not sacrifice the linearity of the molecule. This modification stabilizes the hydrate form sufficiently to enable its isolation as evident from the appearance of the carbonyl carbon as a quartet at 94.3 ppm in the ¹³C NMR spectrum of the TFPA analogue 10c (FIG. 3C). In contrast, the pyruvic amide 5c without the electron withdrawing TFM group was isolated in its keto-form with the carbonyl carbon appearing as a singlet at 197.2 ppm (FIG. 3A). TFMKs, due to their high electrophilicity, can act as highly reactive covalent warheads and lead to off-target effects, thus limiting their application in drug design. An added advantage of the conversion of TFMKs to TFPAs is that stabilization of the hydrate form reduces the potential of the TFMK group to act as a covalent warhead, thereby minimizing their potential off-target effects and making these molecules less likely to act as pan-assay interference compounds (PAINS). To compare the susceptibility of the two groups to nucleophilic addition, the reactivity of the two groups towards addition of the methyl ester of N-Boc cysteine as a thiol-based nucleophile was studied by a ¹⁹F-NMR-based kinetic study. The TFMK showed the appearance of a new fluorine resonance corresponding to the thiol addition product, in addition to the signals corresponding to the ketone and the hydrate forms. In contrast, only a signal corresponding to the hydrate form was observed with the PFPA without any sign of thiol addition product.

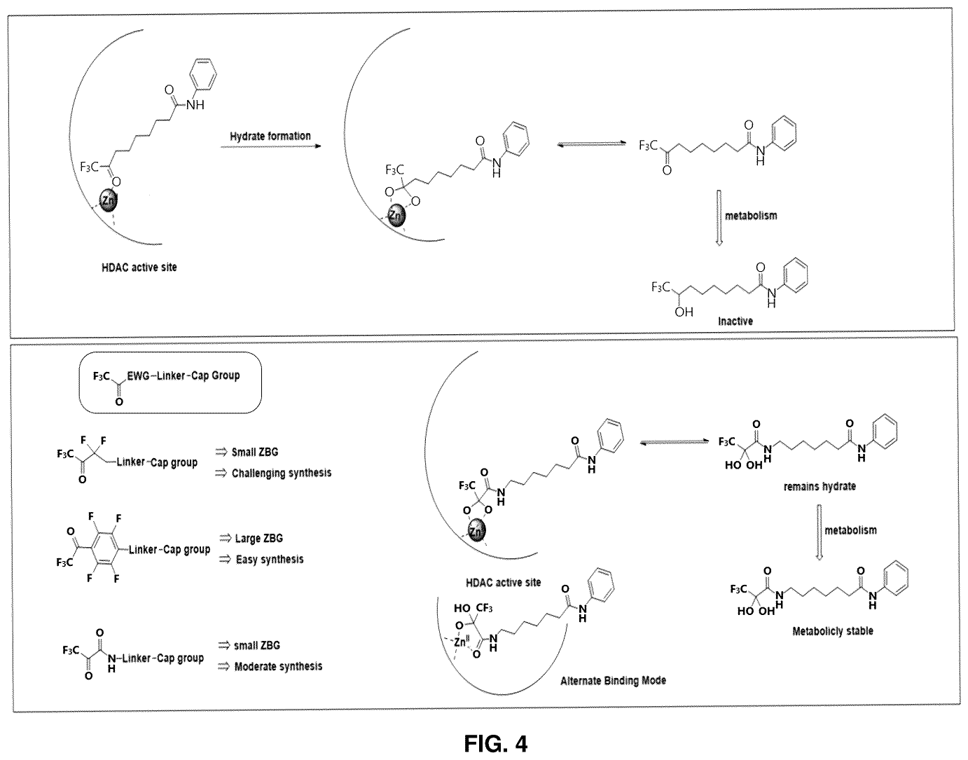

An alternative, and different, design using trifluorolactic amides as HDAC 6-selective inhibitors has been published. However, the mechanism of zinc-binding underlying this alternative design was based on increasing the acidity of the alcohol by the trifluoromethyl group, facilitating its deprotonation, and creating a hydroxamate surrogate that binds to HDAC6 via a 5-membered metallocycle, unlike the 4-membered binding mode of TFPAs involving the gem diol group. However, the mode of binding of the trifluorolactic amides indicates the possibility of an additional binding mode of TFPA ZBG via a less-strained 5-membered metallocycle involving one of the hydrate-alcohol groups and the adjacent amide-carbonyl, in addition to the 4-membered metallocycle involving the two gem-diol hydroxyl groups. (FIG. 4).

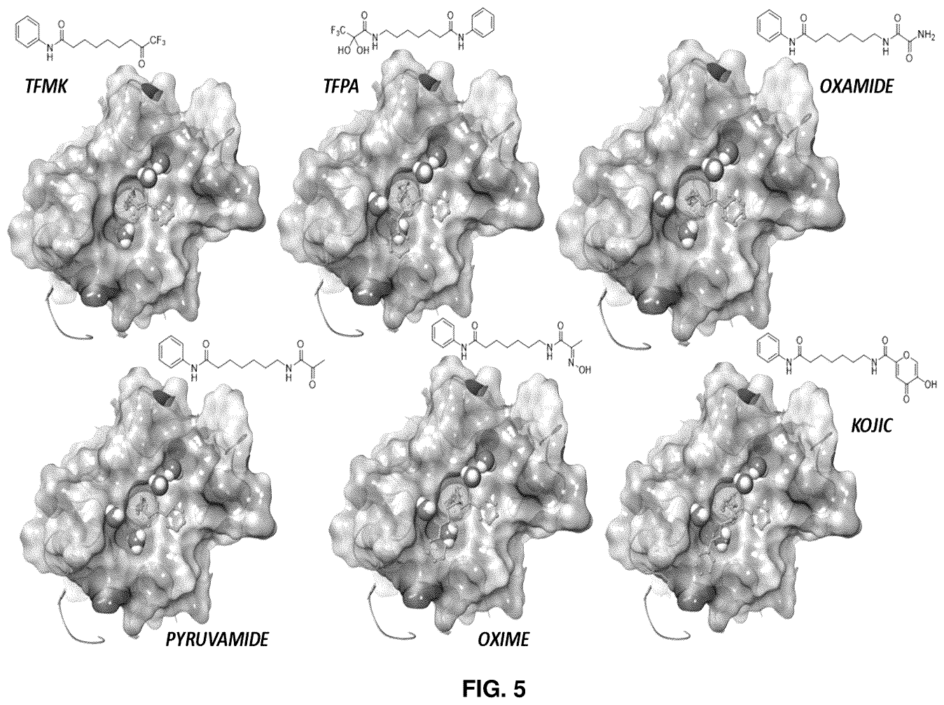

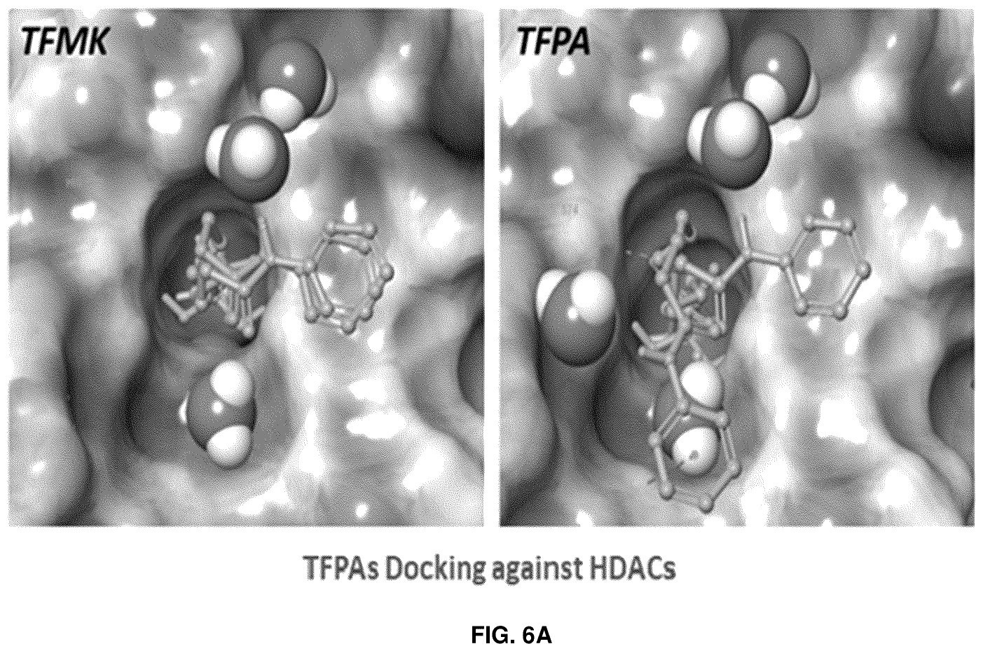

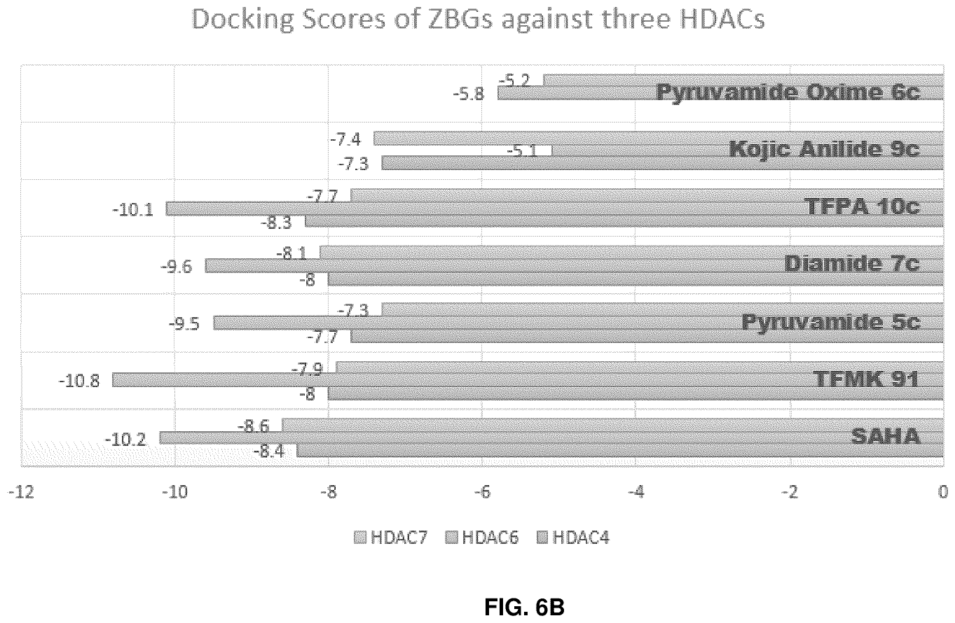

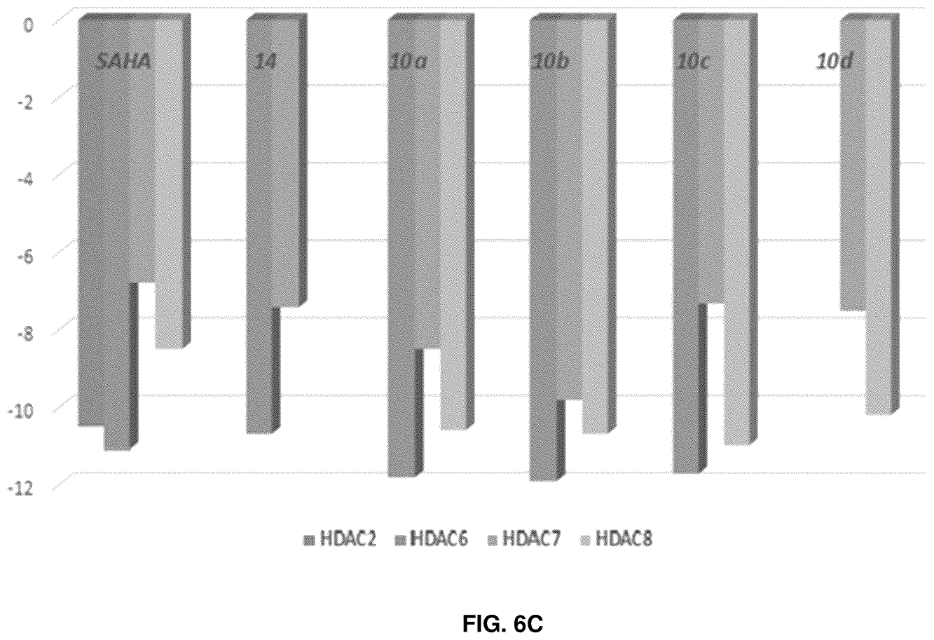

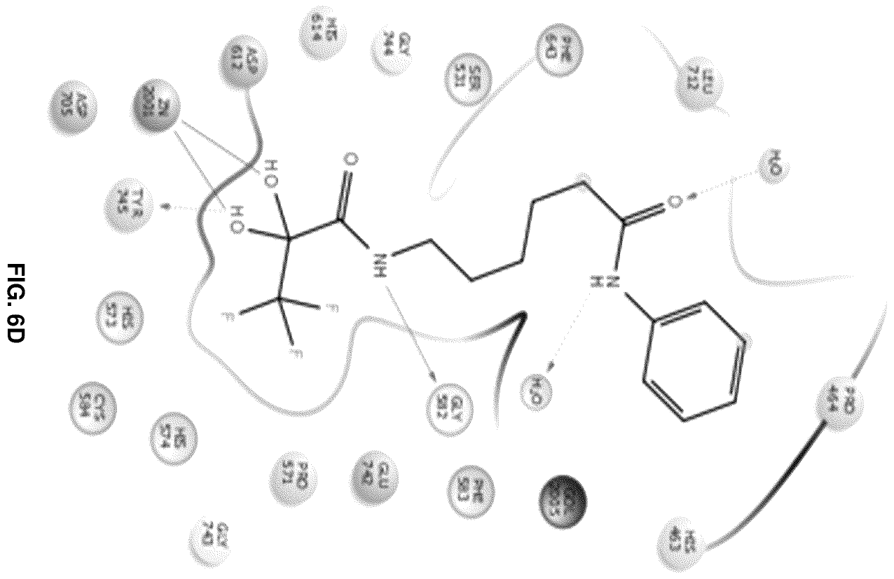

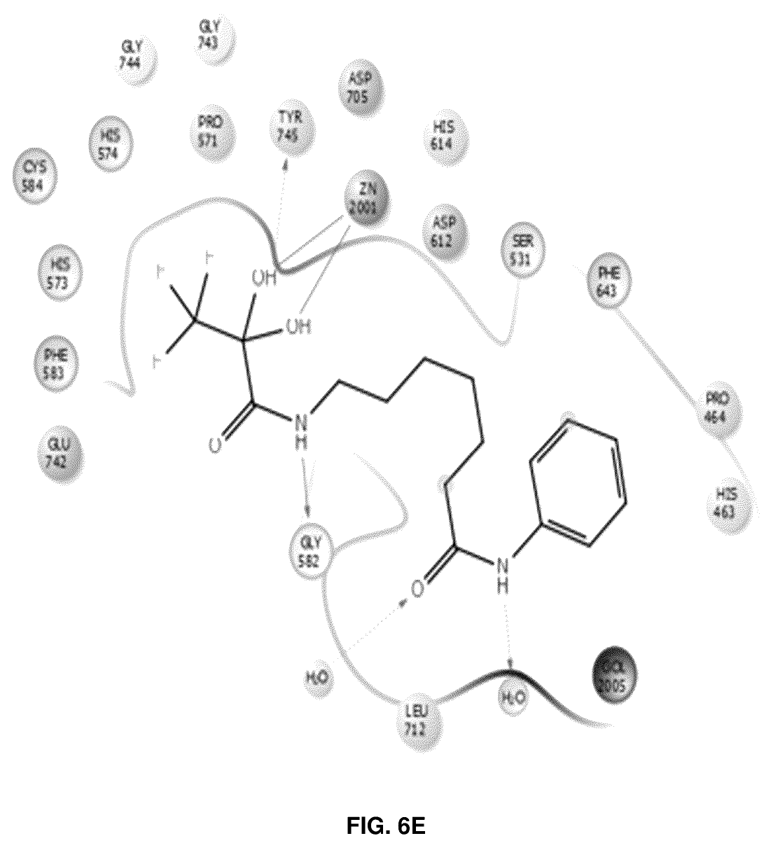

To compare binding modes of the designed SAHA analogues with alternative ZBGs (FIG. 2B), they were docked on several HDACs. They all contained an anilide cap group and 6 methylene carbons as in SAHA. Also included were the SAHA-like TFMK analogue synthesized by others as a reference, along with SAHA for comparison of docking scores and binding poses. It was observed that all the molecules bind to only HDAC4, HDAC6, and HDAC7 in the correct orientation. These three HDACs were used to compare the docking scores (FIG. 6B). The surface representation of each molecule aligned with SAHA inside the HDAC6 binding site is shown in FIG. 5.

All the molecules aligned with SAHA when docked against the designated HDACs with similar binding modes (FIGS. 5, 6A). The docking scores did not reveal any clear preferred candidate for the ZBGs (FIG. 6B). Hence, an in vitro screening of antiproliferative activity was carried out resulting in the identification of 10b as a lead molecule for structure optimization (Table 1). To determine the optimal linker length of 10b, analogues 14 and 10a-10d (Scheme 2) of varying linker length were designed. It was observed that all TFPAs preferred to bind via the gem diol using a four-membered metallocyle transition state, like TFMKs. Docking data revealed that TFPAs bound only to HDAC2, HDAC6, HDAC7, and HDAC8 with minimum error. Results indicated that analogue 10c with a six-carbon linker had the best scores (FIG. 6C). A close examination of the interactions between compounds 10b, 10c and the HDAC6 active site residues (FIGS. 6D-6E) reveals Tyr⁷⁴⁵ as a common denominator. Tyr⁷⁴⁵ is essential for acetyl-lysine cleavage by stabilizing the tetrahedral intermediate formed when a molecule of water, acting as a nucleophile, attacks the acetyl lysine amide bond. Hence, Tyr⁷⁴⁵ interactions with inhibitors can disrupt this process and inactivate the enzyme. Having identified 10b as a lead molecule, a SAR study around the cap group was undertaken. Docking scores of all cap group variants synthesized for SAR study revealed m-bromoanilide TFPA 76 to have better scores than SAHA against HDAC6 and HDAC8, and that it targets Tyr⁷⁴⁵ like 10b.

### Chemistry

### Synthesis

The final products 5a-5c, 7a, 7b, 7d, 8b, 9a-9d, and 10a-10d were synthesized by amide coupling of aminoalkanilides 4a-4d with a ZBG appended to a carboxylic acid group as shown in Scheme 1 (FIG. 7). 1-Ethyl-3-(3-dimethylaminopropyl)carbodiimide (EDCl) and dimethylaminopyridine (DMAP) in dichloromethane were used for amide coupling. However, due to solubility issues, some amines required other coupling agents such as (1-[bis(dimethylamino)methylene]-1H-1,2,3-triazolo[4,5-b]pyridinium-3-oxide hexafluorophosphate (HATU) and 1-hydroxybenzotriazole (HOBt) along with N-methylmorpholine (NMM) in N,N-dimethylformamide (DMF). In the case of kojic anilides 9a-9d (Scheme 2A, FIG. 8), a third method using oxalyl chloride and catalytic DMF was used to generate the final products. Oximes 6a-6c were synthesized from 5a-5c using hydroxylamine hydrochloride in methanol at room temperature.

The only reliable synthetic approach reported for preparation of TFPAs was by an Ugi-like mechanistic pathway using isocyanides and trifluoroacetic anhydride. Considering the high cost of isocyanides needed to make a diverse library of analogues of varying cap groups, simple peptide couplings using amino-alkanamides resembling SAHA fragments were used instead (4a-4d). Peptide coupling using EDCl/DMAP, and EDCl/HOBt failed to give any product. Using HATU, HOBt, and NMM in the presence of molecular sieves under anhydrous conditions at room temperature for four hours resulted in successfully synthesizing TFPAs 10a-10d (Scheme 2A, FIG. 8), albeit in low yields (12-37%).

The aminoalkanilides 4a-4d required for the above syntheses were made by an approach starting from bromoalkanoic acids 1a-1d (Scheme 2A, FIG. 8). This involved peptide coupling to generate bromoanilides 2a-2d, an S_{N}2 reaction using sodium azide in DMF overnight at 80° C. to generate azidoanilides 3a-3d, followed by reduction by catalytic hydrogenation using palladium on carbon to obtain aminoanilides 4a-4d. The spectral data of all these intermediates are consistent with those previously reported.

For synthesis of the TFPA analogue 14 with a short three-methylene linker (Scheme 2B), more commonly available γ-aminobutyric acid (GABA) 11 was used instead of 4-bromobutanoic acid. Boc-protection of GABA (11) gave compound 12. This was followed by peptide coupling with aniline using EDCl and DMAP in dichloromethane overnight to give compound 13. Finally, a tandem Boc-deprotection/peptide coupling of the γ-aminobutyranilide 13 with trifluoropyruvic acid was used to obtain the TFPA 14 (50% yield). This approach was subsequently used for efficient synthesis of Boc-aminoarenes 17-50. Tandem Boc-deprotection/peptide coupling with trifluoropyruvic acid gave TFPAs 53-85 (4-42% yield) without the need to isolate and characterize the intermediate amines. Compounds 51-52 required for the synthesis of TFPAs 86 and 87, respectively, were prepared from 50 by removal of 2,2,2-trichloroethoxycarbonyl (Troc) protecting group (Scheme 2C). Compound 51 underwent peptide coupling with trifluoropyruvic acid to yield 86, while compound 52 was first O-ethylated by refluxing with ethyl iodide and potassium carbonate in acetone for six hours, followed by concentration in vacuo and peptide coupling like 51 to give 87. Some of the aromatic amines used as cap groups included 2-aminothiazoles. Compounds containing these fragments have shown to possess antibacterial, antimicrobial, and other properties, and one such fragment has been used to synthesize a TFMK derivative before. Thus, thiazole pharmacophores were used to generate potent TFPAs. They were either commercially available, or made using known synthetic methods. The spectral data of the functional group of all TFPAs is in excellent agreement with those previously reported by others in which a ¹³C quartet was seen at 90 ppm (in DMSO-d₆), with a J-value of 32 Hz, corresponding to the hydrate of —COCF₃ carbonyl, with no signals corresponding to the keto-form. The molecules synthesized in these examples displayed similar hydrate quartet at 90-91 ppm (acetone-d₆) or 94-95 ppm (MeOH-d₄) (FIG. 3C and NMR spectra). The mass spectral data too corresponds to the hydrate forms. For use as a reference in biological assays, TFMK 91 was synthesized as described in literature.

### Screening for Cytotoxicity of TFPA-Based Analogs

TABLE 1
Evaluation of effect of different zinc-binding groups on HDAC inhibitory activity
by growth inhibition assay using HCT116 cells at a maximum concentration of 100 μM.

| | HCT116 |
|---|---|
| Compound | IC₅₀ |
| 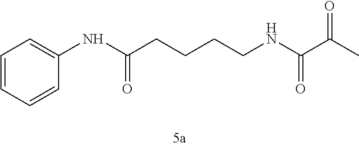 [SMILES: CC(=O)C(=O)NCCCCC(=O)Nc1ccccc1] | >100 |
| 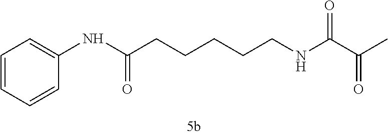 [SMILES: CC(=O)C(=O)NCCCCCC(=O)Nc1ccccc1] | >100 |
| 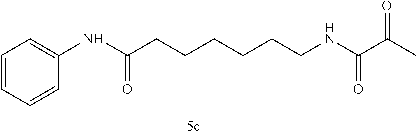 [SMILES: CC(=O)C(=O)NCCCCCCC(=O)Nc1ccccc1] | >100 |
| 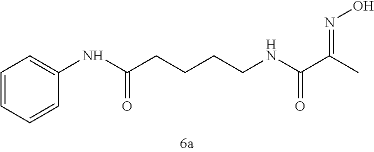 [SMILES: C/C(=N\O)C(=O)NCCCCC(=O)Nc1ccccc1] | >100 |
| 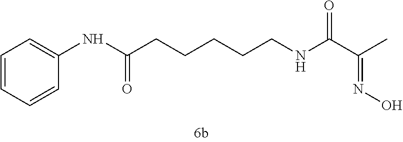 [SMILES: C/C(=N\O)C(=O)NCCCCCC(=O)Nc1ccccc1] | >100 |
| 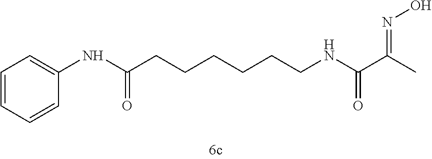 [SMILES: C/C(=N\O)C(=O)NCCCCCCC(=O)Nc1ccccc1] | >100 |
| 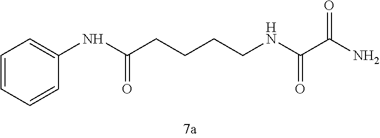 [SMILES: NC(=O)C(=O)NCCCCC(=O)Nc1ccccc1] | >100 |
| 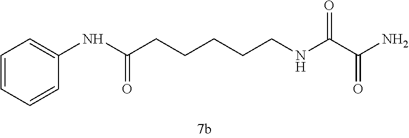 [SMILES: NC(=O)C(=O)NCCCCCC(=O)Nc1ccccc1] | >100 |
| 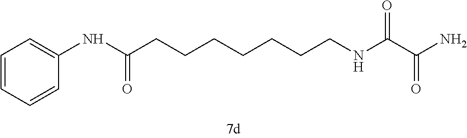 [SMILES: NC(=O)C(=O)NCCCCCCCC(=O)Nc1ccccc1] | >100 |
| 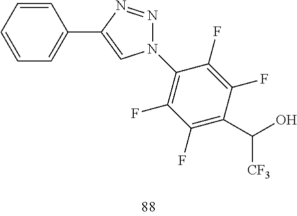 [SMILES: OC(c1c(F)c(F)c(-n2cc(-c3ccccc3)nn2)c(F)c1F)C(F)(F)F] | >100 |
| 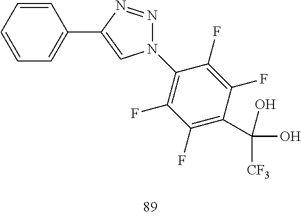 [SMILES: OC(O)(c1c(F)c(F)c(-n2cc(-c3ccccc3)nn2)c(F)c1F)C(F)(F)F] | >100 |
| 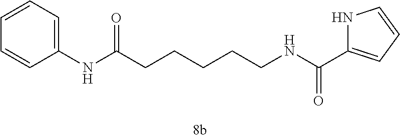 [SMILES: O=C(CCCCCNC(=O)c1ccc[nH]1)Nc1ccccc1] | >100 |
| 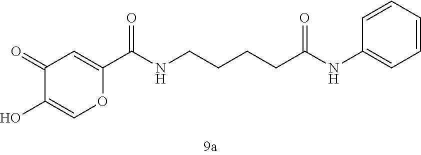 [SMILES: O=C(CCCCNC(=O)c1cc(=O)c(O)co1)Nc1ccccc1] | >100 |
| 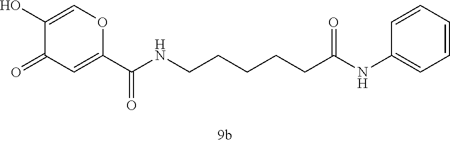 [SMILES: O=C(CCCCCNC(=O)c1cc(=O)c(O)co1)Nc1ccccc1] | >100 |
| 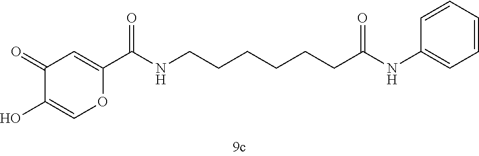 [SMILES: O=C(CCCCCCNC(=O)c1cc(=O)c(O)co1)Nc1ccccc1] | >100 |
| 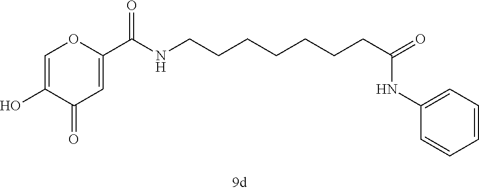 [SMILES: O=C(CCCCCCCNC(=O)c1cc(=O)c(O)co1)Nc1ccccc1] | >100 |
| 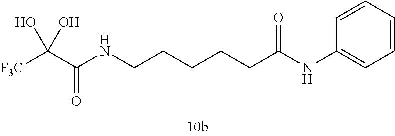 [SMILES: O=C(CCCCCNC(=O)C(O)(O)C(F)(F)F)Nc1ccccc1] | 54.39 ± 6.02 |

Inhibition of growth of HCT116 colon cancer cells at a maximum concentration of 100 μM was used for the initial screening of the ZBGs for HDAC inhibitory potential (Table 1). Included in the assay are the perfluoroaromatic trifluoromethyl ketone 89 and the corresponding trifluoromethyl alcohol 88 (synthesized according to Scheme 1, FIG. 7). All analogues except TFPA 10b were inactive. The lack of activity of compound 89 may possibly be due to the increased steric bulk of the perfluorophenyl group preventing the ZBG from entering the HDAC active site, even though it exists as a stable hydrate. TFPA 10b displayed an IC₅₀ of 54.39 μM and was selected for lead modification.

### Lead Modification

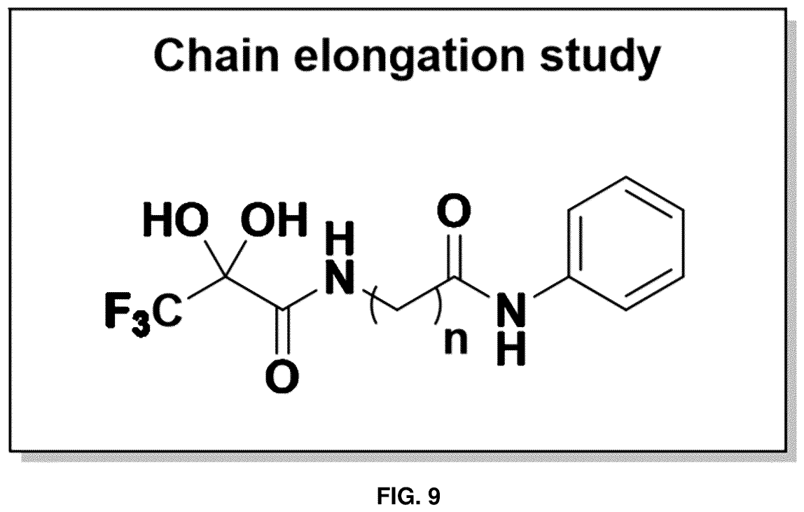

To understand the effect of varying the linker length and to determine the ideal linker length, compounds 10a-10d and 14 were tested against the HCT116 cell line (FIG. 9, Table 2). The results were contrary to the results of the docking studies; compound 10b (n=5), which had the best docking score displayed the lowest activity with an IC₅₀ value of 54.39 μM. Compound 10c (n=6) was the most active with an IC₅₀ value of 9.14 μM against the HCT116 cell line.

TABLE 2
Effect of linker length on growth inhibition of HCT116 cell line

| | IC₅₀ (μM) | IC₅₀ (μM) | IC₅₀ (μM) |
|---|---|---|---|
| Number of CH₂ (n) | 3 | 4 | 5 |
| Compound number | 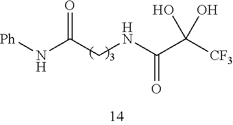 | 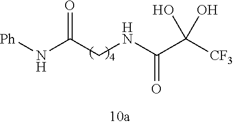 | 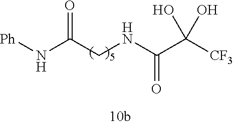 |
| HCT116 | 34.83 ± 5.65 | 20.26 ± 1.23 | 54.39 ± 6.02 |
| | | IC₅₀ (μM) | IC₅₀ (μM) |
| | Number of CH₂ (n) | 6 | 7 |
| | Compound number | 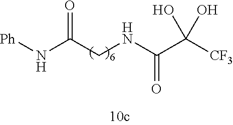 | 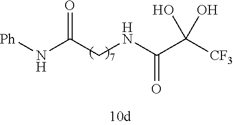 |
| | HCT116 | 9.14 ± 2.25 | 41.67 ± 5.60 |

Having established the validity of the ZBG and the ideal length of the linker, the SAR of the cap group of the most active analogue 10c (n=6) was conducted to further improve its biological activity. This involved the synthesis of a library of analogues 53-87 (Table 3) using cap groups shown in FIG. 10, focusing on variance in the stereoelectronic effects. Their growth inhibitory activity on HCT116 cell line was measured at a maximum concentration of 40 μM (Table 3). SAHA and TFMK analogue 91 used as reference compounds had IC₅₀ values of 1.18 μM and 10.07 μM, respectively (Table 3).

TABLE 3
IC₅₀ values of compounds 53-87 on HCT116 cell line. Compounds 90-92 are
included.

| Cap Group | IC₅₀ |
|---|---|
| 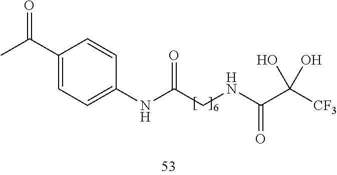 | 12.11 ± 1.82 |
| 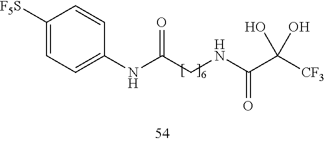 | 9.11 ± 1.32 |
| 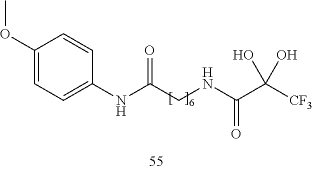 | 4.52 ± 0.63 |
| 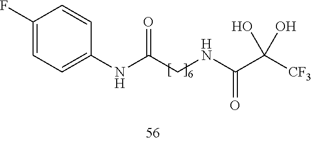 | 7.57 ± 0.37 |
| 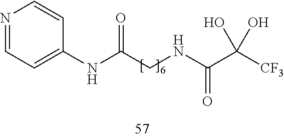 | 6.67 ± 1.02 |
| 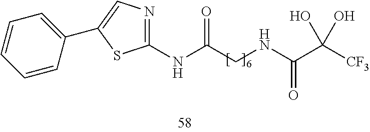 | 2.66 ± 0.32 |
| 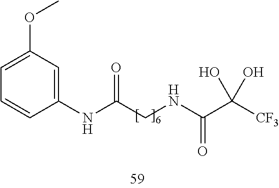 | 6.11 ± 0.72 |
| 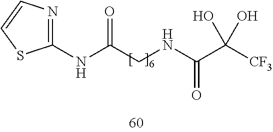 | 10.82 ± 1.28 |
| 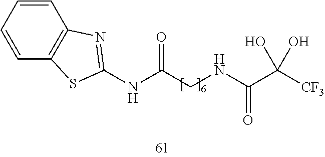 | 2.37 ± 0.23 |
| 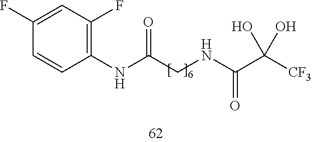 | 18.53 ± 1.29 |
| 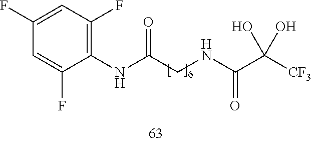 | 22.15 ± 1.97 |
| 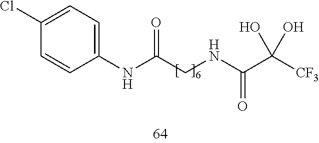 | 4.23 ± 0.25 |
| 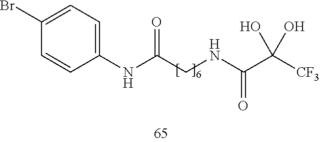 | 3.13 ± 0.17 |
| 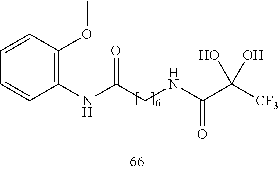 | 10.07 ± 0.71 |
| 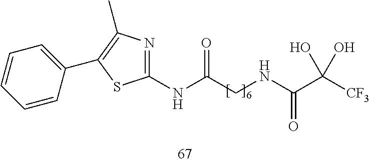 | 10.13 ± 1.2 |
| 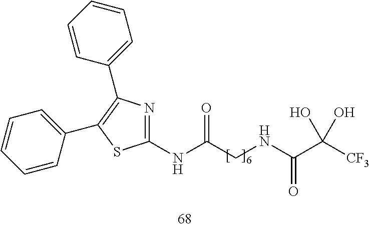 | 29.88 ± 5.63 |
| 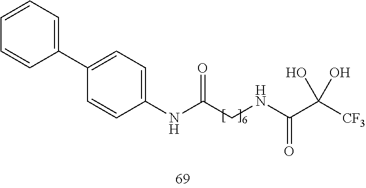 | 4.73 ± 0.87 |
| 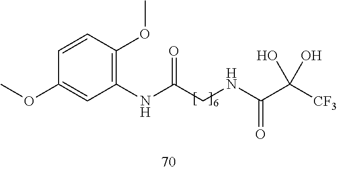 | 3.87 ± 0.15 |
| 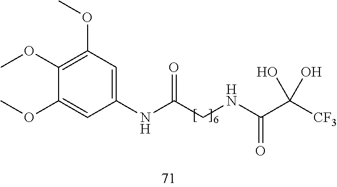 | 14.07 ± 0.83 |
| 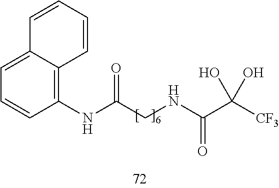 | 6.48 ± 0.75 |
| 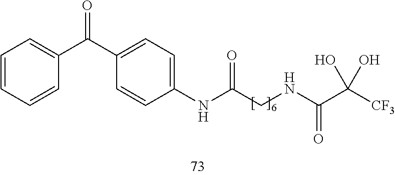 | >40 |
| 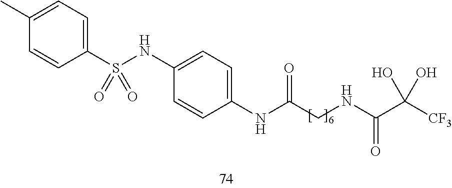 | 17.20 ± 1.69 |
| 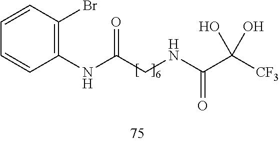 | 14.5 ± 1.02 |
| 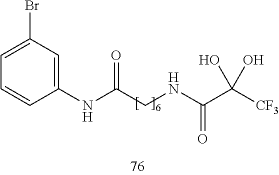 | 3.06 ± 0.63 |
| 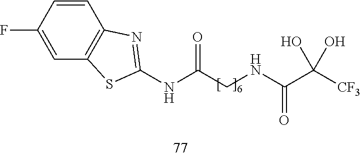 | 6.47 ± 1.18 |
| 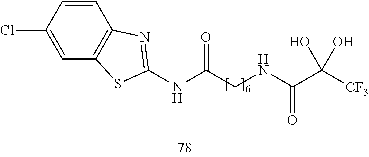 | 10.99 ± 1.29 |
| 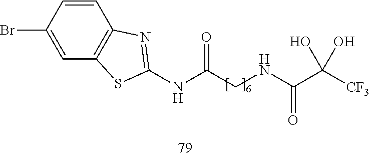 | 10.29 ± 1.36 |
| 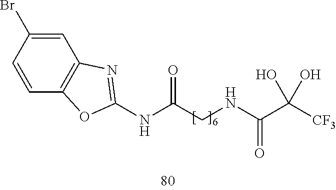 | 12.05 ± 1.47 |
| 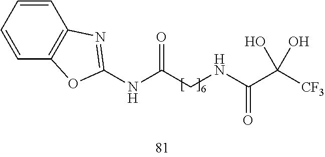 | 3.45 ± 0.36 |
| 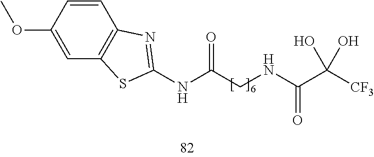 | 7.56 ± 0.73 |
| 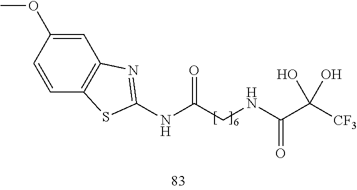 | 2.18 ± 0.32 |
| 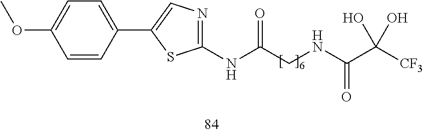 | 0.93 ± 0.14 |
| 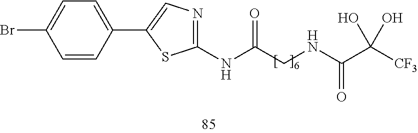 | 2.46 ± 0.25 |
| 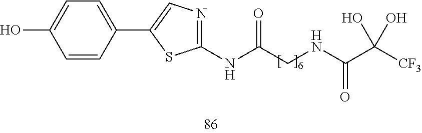 | 1.02 ± 0.07 |
| 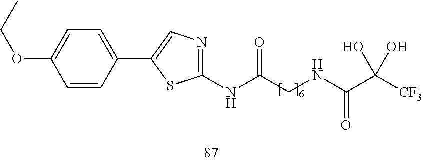 | 4.05 ± 0.87 |
| 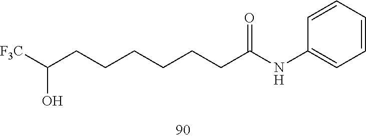 [SMILES: O=C(CCCCCCC(O)C(F)(F)F)Nc1ccccc1] | >40 ¹¹ |
| 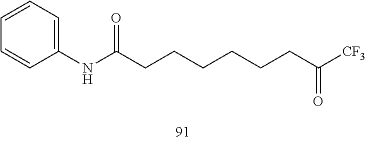 [SMILES: O=C(CCCCCCC(=O)C(F)(F)F)Nc1ccccc1] | 10.07 ± 1.22 |
| 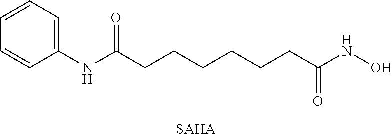 [SMILES: O=C(CCCCCCC(=O)Nc1ccccc1)NO ; "SAHA"] | 1.18 ± 0.07 |
| 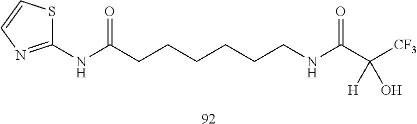 [SMILES: O=C(CCCCCCNC(=O)C(O)C(F)(F)F)Nc1nccs1 ; "92"] | >40 |

Of the analogues substituted at the para position of aniline, p-bromoanilide 65 (3.13 μM) and p-methoxyanilide 55 (4.52 μM) had the lowest IC₅₀ values. Hence, the effect of positional substitutions with bromine and methoxy at other positions was also investigated. There was hardly any difference in the IC₅₀ values of meta-bromo (3.06 μM) and para-bromo (3.13 μM) analogues, but ortho-bromo substitution 75 increased the IC₅₀ value to 14.5 μM. A direct decreasing trend in activity was observed with the methoxy group going from 4.52 μM for para (55) to 6.11 μM for meta (59) and 10.07 μM for ortho (66). Among the bulkier aromatic groups, the best results were obtained for the 5-phenylthiazole variant 58 (2.66 μM). This is consistent with phenylthiazole TFMKs being among the most active variants. Therefore, the investigation of SAR of thiazoles was expanded in much greater detail. 4- and 5-positional substitution variants of thiazoles were synthesized, starting with phenyl, methyl phenyl, and diphenyl substituents. The 4,5-diphenyl thiazole was of particular interest as a similarly substituted oxazole fragment existed as a part of the HDAC6-selective inhibitor Tubacin cap group. However, it was found that the 4-position substitution was detrimental for activity, as the 4,5-diphenylthiazole 68 had drastically reduced activity (29.88 μM).

The effect of fusing the thiazole ring with a phenyl group as in benzothiazole vs the phenylthiazole was then investigated. The results were similar with IC₅₀ of 2.37 μM for benzothiazole 61 vs. 2.66 μM for 5-phenylthiazole 58. The effects of substitution at the para-position of phenylthiazoles, and substitutions at 5- and 6-positions of benzothiazoles, were then investigated. Accordingly, p-bromo (85), p-hydroxy (86), p-methoxy (84), and p-ethoxy (87) phenylthiazoles were synthesized. Of these, the p-methoxyphenylthiazole 84 produced an IC₅₀ of 0.93 μM, making it more potent than SAHA (IC₅₀ 1.18 μM). Replacing the methoxy with an ethoxy in 87 reduced the activity to IC₅₀ 4.05 μM, while the corresponding free phenol analogue 86 had an IC₅₀ of 1.02 μM, almost as active as the corresponding methoxy analogue 84. For the benzothiazole series, 6-fluoro (77), 6-chloro (78), 6-bromo (79), 5-methoxy (83), and 6-methoxy (82) benzothiazoles were synthesized. Of these, only the 5-methoxybenzothiazole variant 83 (2.18 μM) produced better results than the unsubstituted parent molecule.

As metabolic reduction of TFMKs has been shown to abolish their ability to kill cancer cells, it was important to test if reduction of TFPA would produce the same effect. TFPA 60 was reduced to the corresponding trifluoromethyl-α-hydroxyamide (TFMHA) 92. It was inactive on the HCT116 cell line (Table 3).

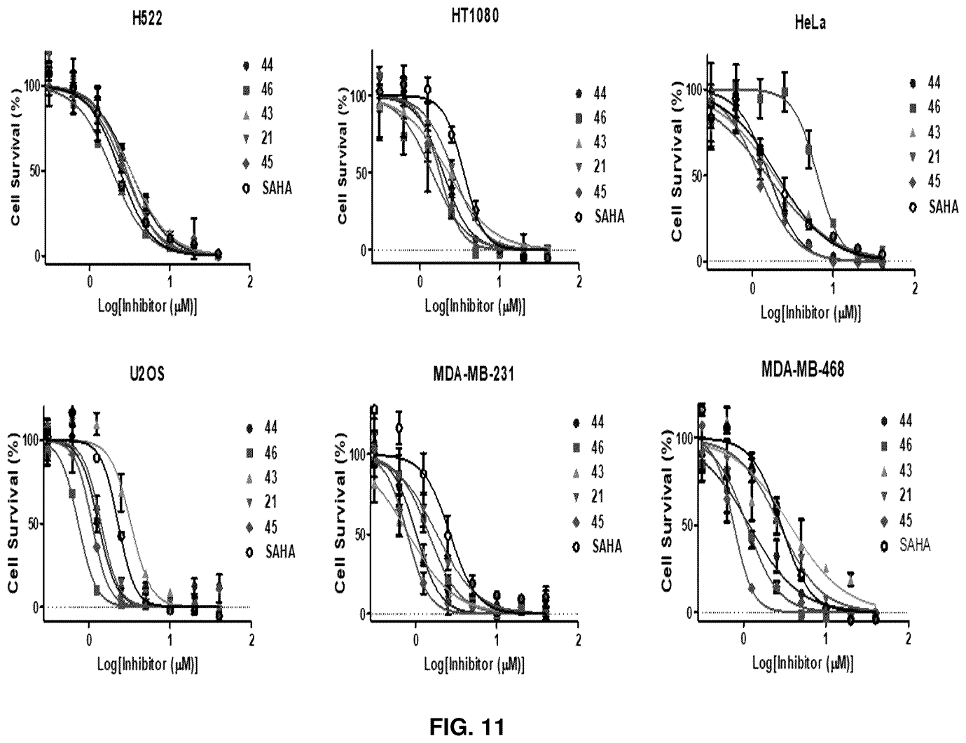

After screening the activity of the compounds against the HCT116 cell line, the five compounds with the lowest IC₅₀ values (compounds 61, 83, 84, 85, and 86) were tested on additional cell lines, namely, the NCI-H522 lung cancer cell line, HT1080 fibrosarcoma cell line, HeLa cervical cancer cell line, U2OS osteosarcoma cell line, and MDA-MB-231 and MDA-MB-468 breast cancer cell lines. SAHA was used as a positive control for comparison. All compounds showed comparable or better growth inhibitory activity than SAHA on some cell lines, while compounds 84 and 85 displayed better activity than SAHA against most of the cell lines (FIG. 11, Table 4).

TABLE 4
IC₅₀ values of five compounds against multiple cell lines,
as compared to SAHA. Compound 85 is highlighted in bold.

| Cell Line | Compound 84 | Compound 86 | Compound 83 | Compound 61 | Compound 85 | SAHA |
|---|---|---|---|---|---|---|
| H522 | 3.24 ± 0.56 | 1.90 ± 0.21 | 3.23 ± 0.41 | 2.86 ± 0.36 | 2.70 ± 0.34 | 2.38 ± 0.28 |
| HT1080 | 1.96 ± 0.21 | 1.42 ± 0.29 | 2.28 ± 0.47 | 2.65 ± 0.21 | 1.76 ± 0.22 | 3.56 ± 0.30 |
| HeLa | 1.60 ± 0.12 | 6.38 ± 0.92 | 1.86 ± 0.31 | 1.52 ± 0.23 | 1.22 ± 0.09 | 2.04 ± 0.31 |
| U2OS | 1.31 ± 0.18 | 0.74 ± 0.04 | 3.29 ± 0.46 | 1.41 ± 0.18 | 1.09 ± 0.09 | 2.23 ± 0.15 |
| MDA-MB-468 | 1.16 ± 0.26 | 1.10 ± 0.10 | 3.65 ± 0.11 | 1.31 ± 0.18 | 0.76 ± 0.08 | 2.85 ± 0.37 |
| MDA-MB-231 | 1.02 ± 0.09 | 1.46 ± 0.15 | 0.81 ± 0.10 | 1.83 ± 0.35 | 0.79 ± 0.10 | 2.23 ± 0.15 |

Ten selected compounds were then tested in NCI 60 cell one-dose assay, at a single concentration of 10 μM, followed by a five-dose assay at five different concentrations for eight of the ten compounds that displayed sufficient cytostatic/cytotoxic activity at 10 μM. The one-dose assay displays mean growth percentages, while the five-dose assay shows GI₅₀, TGI, and LC₅₀ values (Table 5). Most of the compounds demonstrated potent cytostatic activity, with several of them (58, 84, 85) showing cytotoxic effects as well (Table 5). Interestingly, compound 85 exhibited the least mean growth percentage and GI₅₀ value, concurring with the previous assay results (Table 4). Even though compound 84 was the most active on the HCT116 cell line, the results on both six additional cell lines (FIG. 11, Table 4) and the NCI-60 cell one-dose and five-dose assays (Table 5) showed that compound 85 had a better activity profile against multiple cell lines, making it more consistent. Hence, compound 85 was picked as the lead inhibitor in the study.

TABLE 5
NCI data of ten compounds against sixty cell lines displaying mean cell growth percentage
(one-dose assay), GI₅₀, TGI, and LC₅₀ values (five-dose assay). Positive growth
percentages indicate cytostatic activity, while negative percentages imply cytotoxicity.
The best results are highlighted in bold. ND—Not Determined. GI₅₀ values correspond
to the drug concentration needed to prevent 50% cell growth. TGI (Total Growth Inhibition)
values correspond to the concentrations needed to prevent complete cell growth.
LC₅₀ values are a measure of cytotoxicity, corresponding to the minimum concentrations
needed to kill 50% of the target cells.

| Compound | 84 | 85 | 83 | 70 | 61 | 64 | 65 | 69 | 58 | 55 |
|---|---|---|---|---|---|---|---|---|---|---|
| Mean | −10.17 | −11.47 | −4.91 | 35 | −2.45 | 15.99 | 2.58 | 2.1 | −10.06 | 37.09 |
| Growth % | | | | | | | | | | |
| GI₅₀ (μM) | 1 | 0.93 | 1.23 | ND | 3.39 | 5.13 | 3.16 | 3.09 | 3.02 | ND |
| TGI (μM) | 8.51 | 11.4 | 12.3 | ND | 21.37 | 26.92 | 20.89 | 15.14 | 16.98 | ND |
| LC₅₀ (μM) | 26.3 | 26.3 | 28.18 | ND | 28.84 | 35.48 | 31.62 | 29.51 | 22.91 | ND |

### Isoform Selectivity Testing

The inhibitory potencies of the compounds were tested against HDAC1, HDAC2, HDAC3, HDAC4, HDAC6, and HDAC8. The overall results indicated that all the inhibitors possess an affinity for class 1 HDACs over class II HDACs. The dose dependent curve data showed that the compounds displayed higher potency against HDAC8, with IC₅₀ values in the low nanomolar range (Table 6). The most potent compound was 69 with an IC₅₀ of 4.1±0.6 nM against HDAC8. The most selective compounds were 65, 83, and 58 (Table 6). However, compound 83 is the standout inhibitor, exhibiting between 11-to-460-fold selectivity for HDAC8, when all three were compared using heat maps of the ratios of IC₅₀ values against different HDACs. Hence, compound 83 may be HDAC8 selective. However, a direct correlation between HDAC 8 selectivity and cell growth inhibitory activity was not observed. The results of the assay may not be enough to justify HDAC isoform selectivity, as HDACs exist as multiprotein complexes at a cellular level. In this context, it is important to note that selective HDAC 6 inhibitors have been shown to display anticancer properties only at high concentrations resulting in low selectivity and off-target effects.

TABLE 6
Inhibitory potencies against HDAC1, HDAC2, HDAC3, HDAC4, HDAC6,
and HDAC8. Highlighted in bold are the most selective inhibitors.

| | IC₅₀ (nM) | | | | | |
|---|---|---|---|---|---|---|
| Compound | HDAC1 | HDAC2 | HDAC3 | HDAC4 | HDAC6 | HDAC8 |
| 65 | 120 ± 20 | 3200 ± 300 | 230 ± 20 | 2600 ± 200 | 100 ± 10 | 11 ± 2 |
| 61 | 56 ± 3 | 580 ± 20 | 210 ± 10 | 430 ± 30 | 82 ± 7 | 5.3 ± 1.6 |
| 85 | 120 ± 10 | 2400 ± 100 | 59 ± 1 | 1700 ± 200 | 35 ± 4 | 10 ± 1 |
| 69 | 61 ± 4 | 1800 ± 100 | 72 ± 2 | 1100 ± 100 | 38 ± 3 | 4.1 ± 0.6 |
| 55 | 100 ± 10 | 1400 ± 100 | 180 ± 10 | 1600 ± 200 | 440 ± 20 | 51 ± 5 |
| 86 | 110 ± 10 | 3600 ± 300 | 99 ± 9 | 2300 ± 300 | 210 ± 20 | 30 ± 3 |
| 64 | 160 ± 10 | 4900 ± 200 | 270 ± 20 | 2700 ± 300 | 260 ± 50 | 52 ± 7 |
| 83 | 4100 ± 100 | 2500 ± 400 | 120 ± 10 | 1900 ± 400 | 100 ± 10 | 8.9 ± 0.7 |
| 58 | 180 ± 10 | 4800 ± 300 | 620 ± 50 | 2500 ± 300 | 350 ± 20 | 21 ± 3 |
| 84 | 97 ± 2 | 360 ± 40 | 100 ± 10 | 2500 ± 100 | 37 ± 10 | 13 ± 1 |
| SAHA | 33 ± 1 | 96 ± 10 | 20 ± 1 | 12000 ± 800 | 33 ± 3 | 540 ± 10 |

Potencies were determined using a chemiluminescent HDAC activity assay, with the mean and standard error of at least three independent trials shown. The IC₅₀ values for SAHA were previously reported using the same assay.

### Hyperacetylation Assay

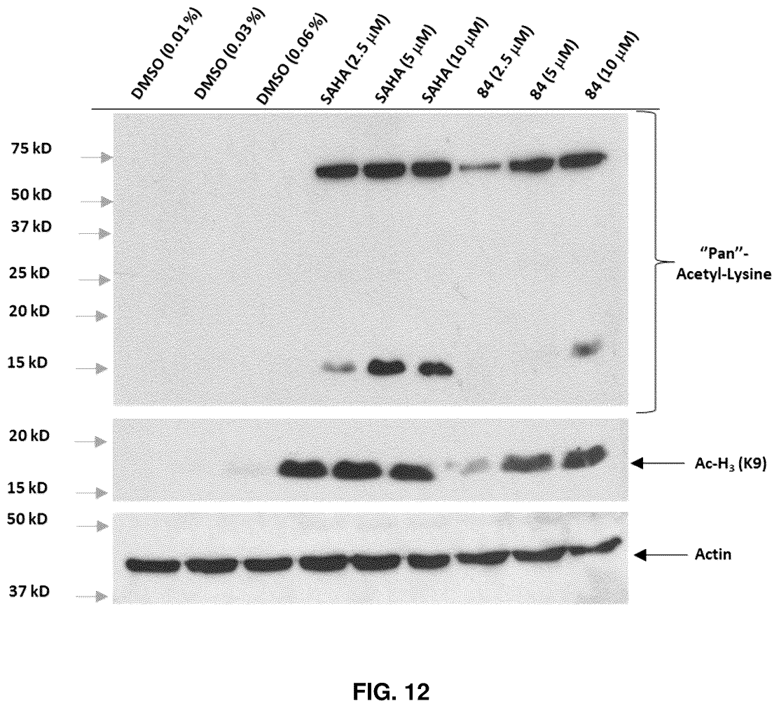

The hallmark of HDAC inhibition is the hyperacetylation of several proteins which are substrates for HDACs. Thus, histone H3 hyperacetylation assay of some active compounds was carried out by western blot using antibodies for Histone H3 and pan-acetyl lysine. When tested at concentrations of 2.5, 5, and 10 μM, both acetyl-H3 and “pan” acetyl-lysine (FIG. 12) demonstrated elevated levels in a similar way to SAHA, confirming that these molecules target HDACs.

### Evidence of Apoptosis

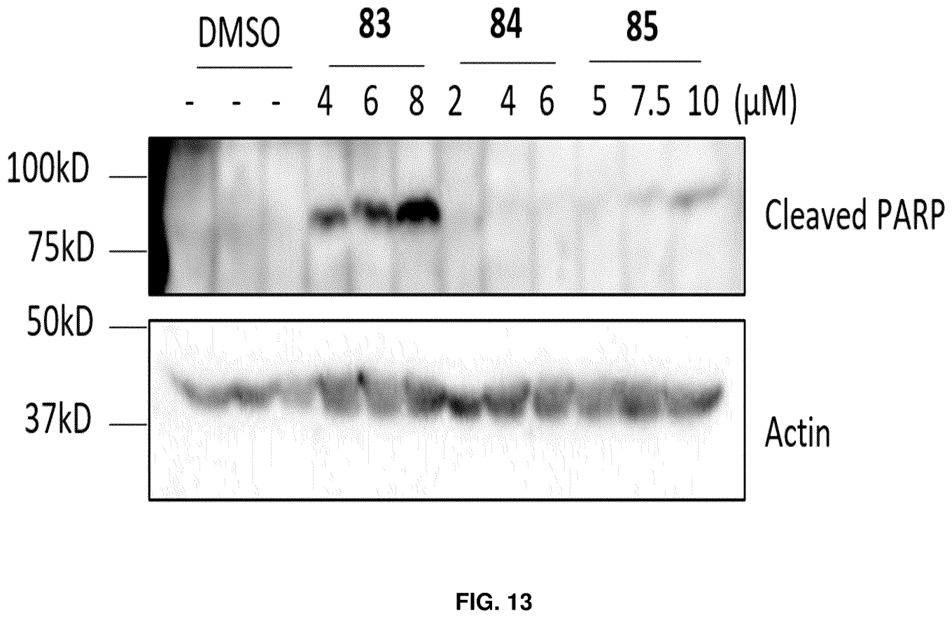

Pan HDACi SAHA has been reported to induce apoptosis in a caspase 8 dependent manner in HCT116 cells. To determine whether the compounds herein induced apoptosis, the enzyme Poly (ADP ribose) polymerase 1 (PARP1) was analyzed by western blotting. Caspase-dependent proteolytic cleavage of PARP1 is one of the hall marks of apoptosis and antibodies that specifically recognize the cleavage products are available. Western blot analysis indicated that the HDACi induced PARP1 cleavage, indicating induction of apoptosis (FIG. 13). HCT116 cells were treated with compounds 83, 84, and 85 for 24 h. Dose-dependent elevation of cleaved PARP1 was detectable after exposure to 83 and 85, but not compound 84. Absence of PARP1 cleavage in cells exposed to 84 may be a result of insufficient dose or time of treatment. The fact that 83 and 85 did induce PARP1 cleavage indicates that these analogues kill cells through an apoptotic mechanism.

### Selectivity for Cancer vs Normal Cells

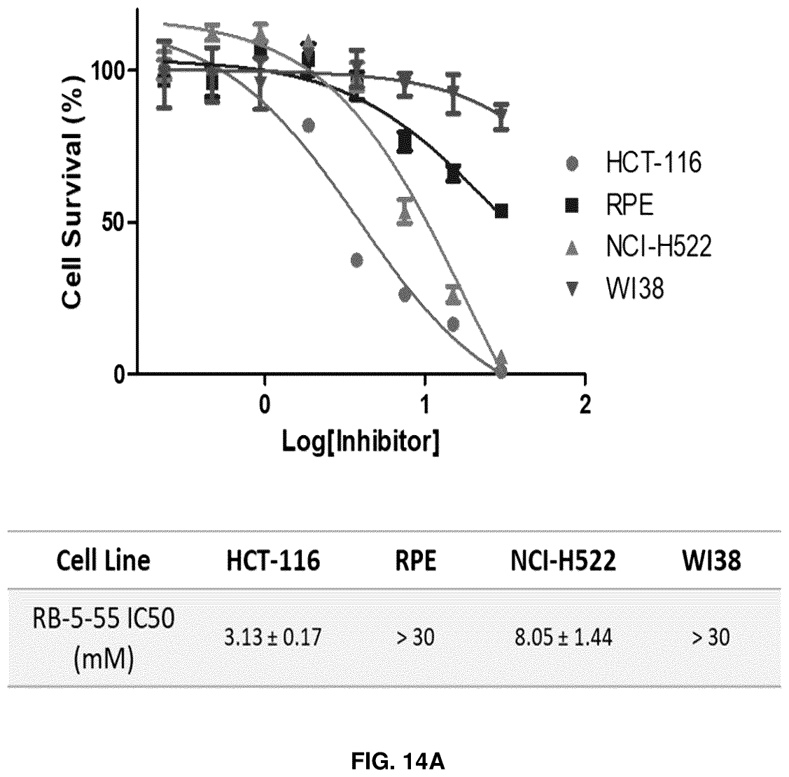

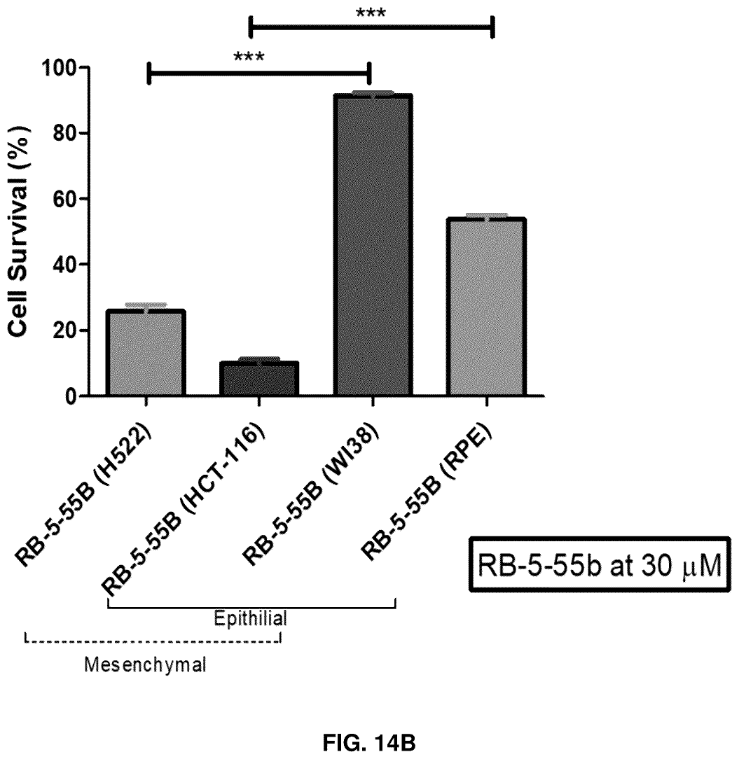

To determine the selectively of the inhibitors for cancer cells over normal cells, compound 55 was tested on the mesenchymal cancer cell line NCI-H522 and the mesenchymal normal cell line WI38, as well as the epithelial cancer cell line HCT116 and the epithelial normal cell line RPE (FIGS. 14A-14B). The cancer cell lines were found to be more sensitive to the compound compared to normal cells. Moreover, at a maximum concentration of 30 μM, the NCI-H522 cells displayed above 20% survival while the HCT116 cells showed close to 10% cell survival. In comparison, the WI38 cells displayed greater than 80% survival while the RPE cells show more than 50% survival (FIG. 14B). Also, the IC₅₀ values of 55 against NCI-H522 and HCT116 cell lines were 8.05 μM and 3.13 μM, but against RPE and WI38 they were inactive (>30 μM, FIG. 14A). This reaffirms the selectivity of TFPAs for cancer cells over normal cells.

### Metabolism Studies

Previous studies established that trifluoromethyl ketones are metabolically unstable and reduced in vivo to trifluoromethyl alcohols (TFMAs). This metabolic reduction caused a major loss in activity. The TFPAs possess a TFMK moiety, albeit with significantly enhanced electrophilicity. It was believed that in TFPAs, the hydrate form of the ketone is stabilized compared to TFMKs, and is therefore less susceptible to reductive metabolism. To prove the superior metabolic stability of the TFPAs over TFMKs, a metabolic stability assay was carried out. Metabolic stability studies are primarily conducted using liver microsomal assays, as the predominant cytochrome P450 enzymes present in them are responsible for the metabolism of most xenobiotics in the human body. However, P450 transformations are mostly oxidative in nature, precluding their use to assess the stability of ketone drugs against reductive metabolism. A comprehensive review of ketone drug metabolism showed the presence of carbonyl reductases and additional reductases mainly in the liver cytosol, and a minor amount in microsomes. Thus, using a liver extract containing both cytosol and microsomal enzymes would be more appropriate for this assay. This was accomplished by using the S9 fraction, which is basically the supernatant obtained from centrifugation of hepatocytes at 9000 g and contains both liver cytosol and microsomal enzymes. LC-ESI-MS was used to monitor the reduction of TFPA 60 over time and it was compared with TFMK 91.

TFPAs undergo the same transformation on incubation with cells or whole blood. The S9 fraction (which contains liver cytosol and microsomal enzymes) were used for metabolism studies, and metabolism was monitored by LC-MS. The TFMK peak disappeared in 15 minutes with a TFMK alcohol peak appearing simultaneously, whereas the TFPA peak did not disappear even after 2 hours and no TFPA alcohol peak being observed.

### Metabolic Stability of TFPAs vs TFMKs

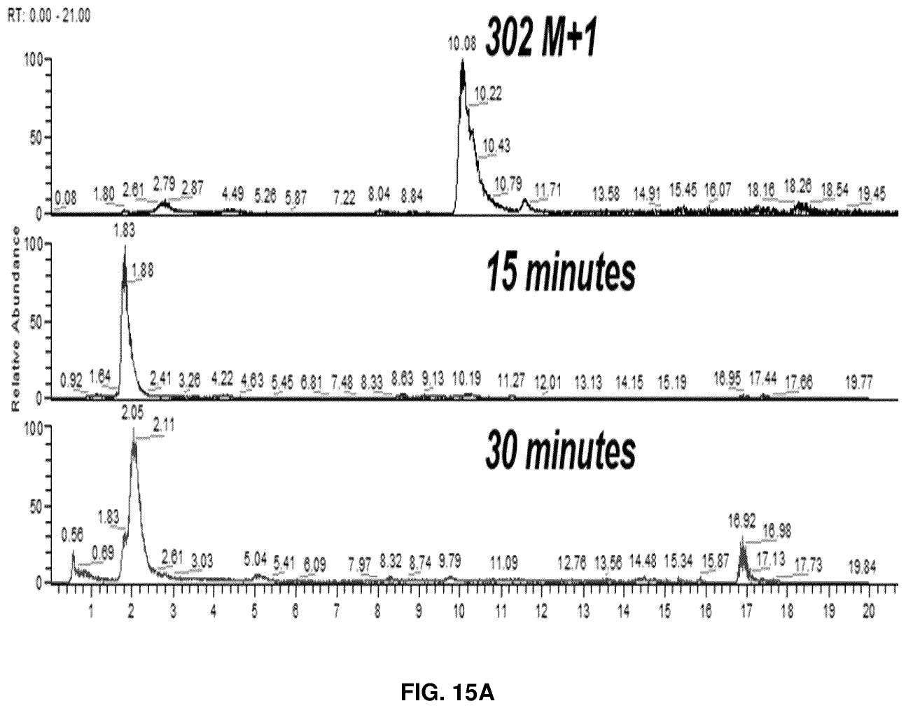

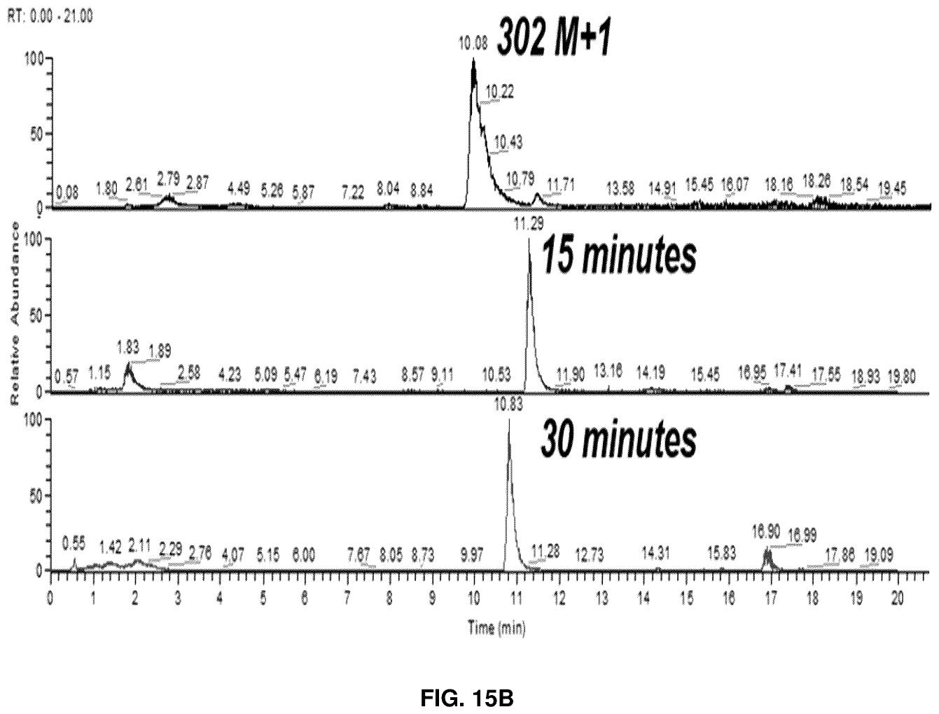

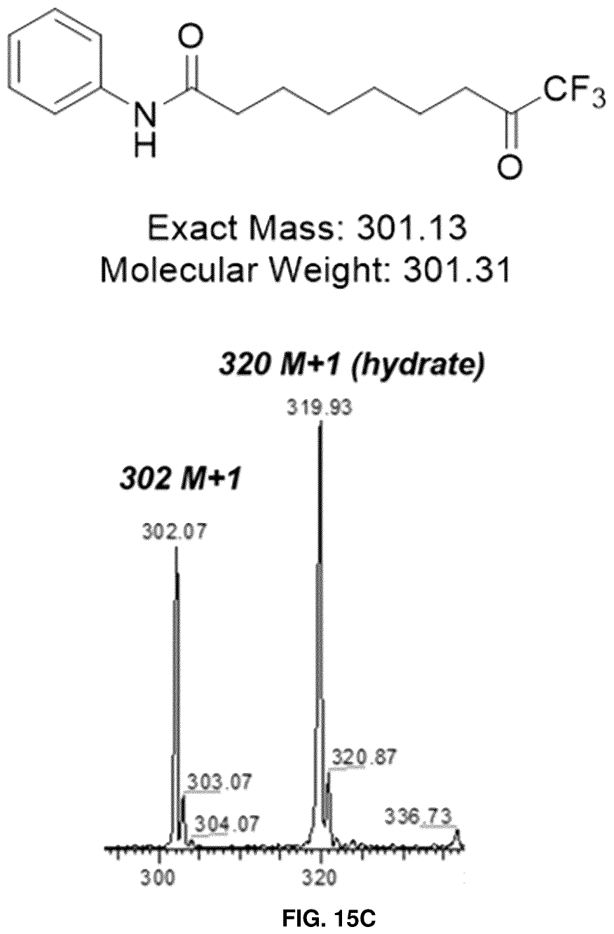

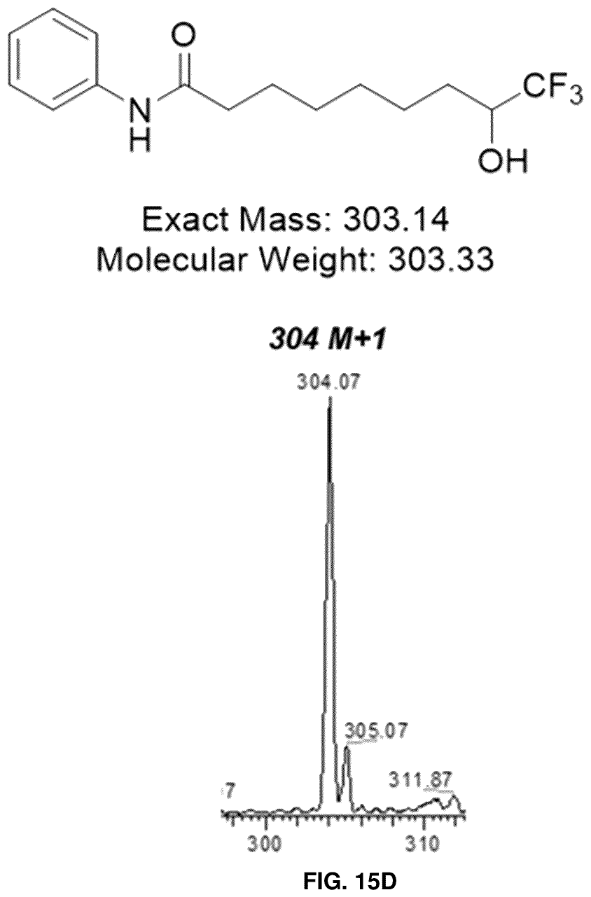

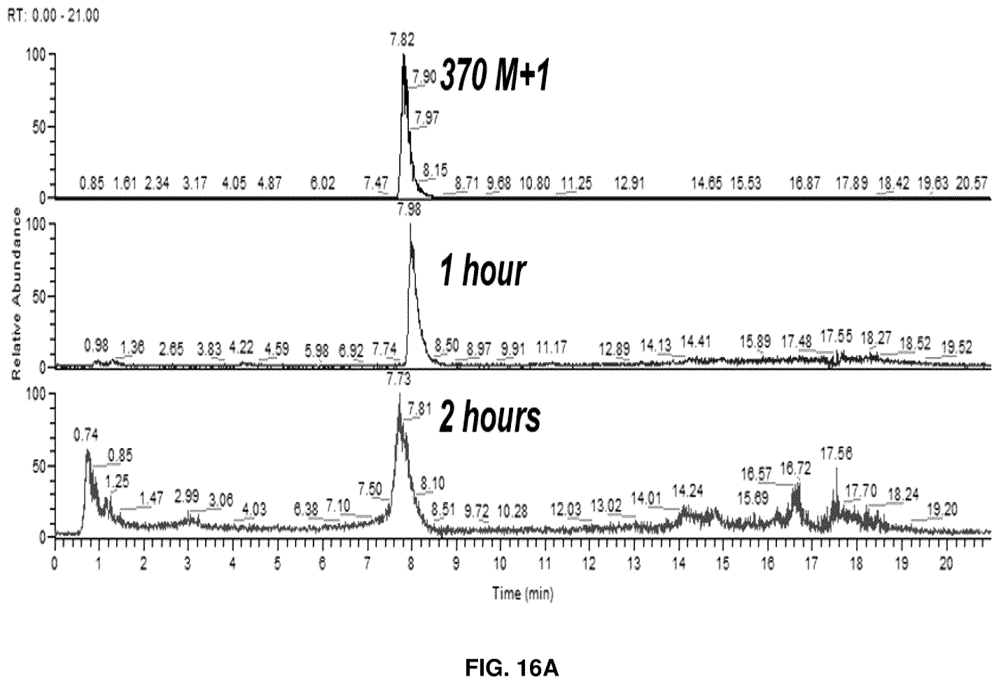

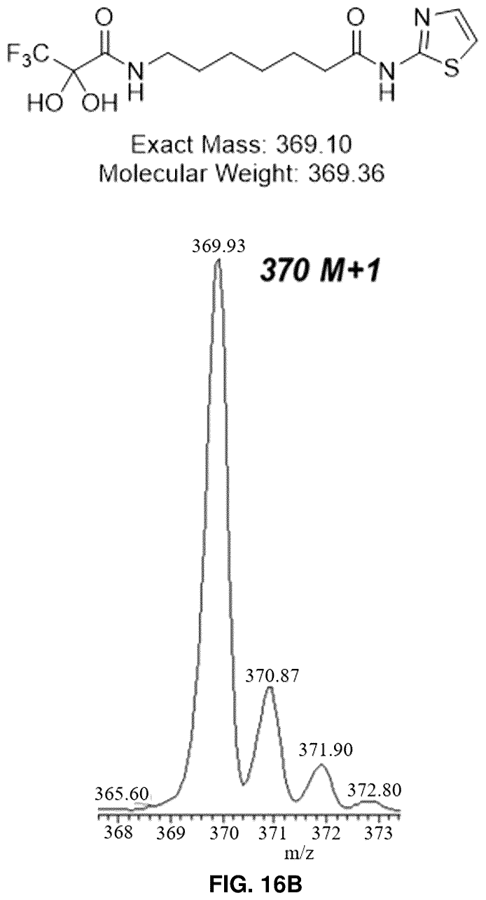

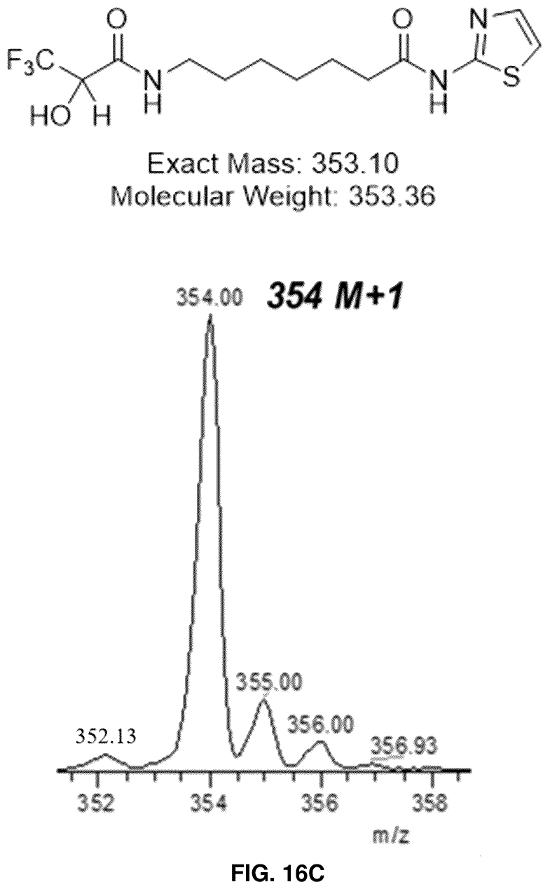

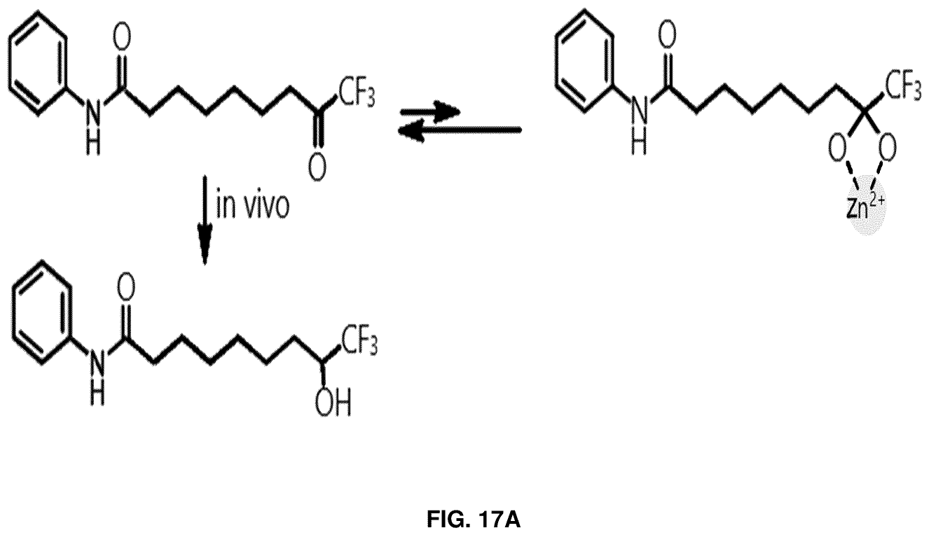

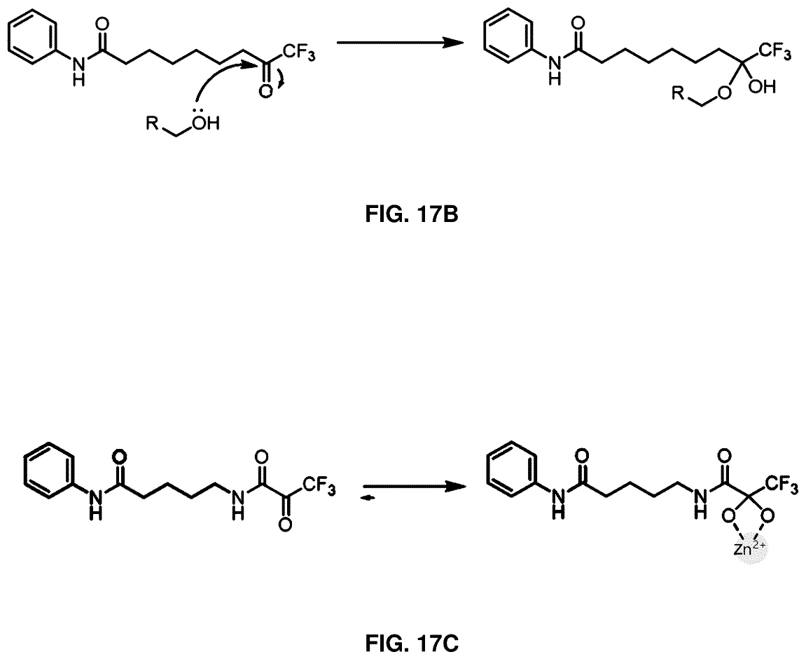

The disappearance of TFMK 91 m/z 302 (FIG. 15C) at 15-minute intervals (FIG. 15A) was tracked, and the TFPA 60 m/z 370 M+1 peak was monitored over a period of 2 hours (FIG. 16A).

To test the rapid kinetics of TFMK metabolism, the aliquots obtained from the S9 fraction assay for TFMK 91 were analyzed. The M+1 peak of m/z 302 completely disappeared in 15 minutes (FIG. 15A). Simultaneously, the formation of the TFMA 90 M+1 peak of m/z 304 (FIG. 15D) was monitored over 30 minutes (FIG. 15B). It was formed in 15 minutes, just around the same time as the disappearance of compound 91. Hence, the LC-MS results show a quantitative biological reduction of TFMK 91 by metabolizing enzymes in 15 minutes.

In contrast, it was observed that TFPA 60 was resistant to reducing conditions imposed by the S9 fraction; the M+1 of m/z 370 for compound 60 (FIG. 16B) did not disappear over 2 hours (FIG. 16A). Similarly, there were no traces of the M+1 peak of TFMHA 92 at m/z 354 (FIG. 16C) around a retention time of 8.13 minutes, over 2 hours. This proves that there is no reduction to the alcohol taking place. Metabolism is a process executed by the liver to transform non-polar substances into polar products that can be excreted easily. Ketone drugs are generally hydrophobic in nature and metabolic reduction serves to introduce polarity into the molecule for excretion. Since TFPA 60 is more polar than TFMHA 92, it does not require metabolic reduction to increase polarity. This is a stark contrast to the quantitative biological reduction of compound 91 in 15 minutes.

### CONCLUSION

The metabolic reduction of linear TFMKs has been a significant barrier that has prevented them from being considered as successful SAHA-alternatives for HDAC inhibition for 20 years. This was probably due to the inability of the ketone function to sustain the hydrate-form outside the active site of HDACs, making them vulnerable to reductive metabolism by carbonyl reductases in the liver. However, as shown in these examples, this vulnerability may be overcome by structural modification that stabilizes the hydrate form while maintaining the linearity of the functional group such that it is small enough to enter the active site. This modification entails enhancing the electrophilicity of the ketone group to favor its hydrate form and thereby prevent its reductive metabolism. This was successfully achieved by incorporating an additional electron-withdrawing amide group adjacent to the trifluoromethyl carbonyl group, resulting in TFPAs. An additional advantage of this structural modification is that it reduces the potential of the TFMK group to act as a covalent warhead and produce off-target effects. Molecular docking confirmed that the designed molecules can bind to several HDAC proteins by chelation of the hydrated pyruvamide group with the zinc ion in the HDAC active site and positioning the anilide cap group at the active site outer rim area. This mode of binding agrees with the binding of other well-known inhibitors such as SAHA. Biochemical studies showed that these compounds were potent cytotoxic agents. SAHA-like linkers containing six methylene groups were found to be advantageous for antiproliferative activity of TFPAs. SAR studies of the cap-group of thiazole-based TFPAs identified several potent variants such as compound 85, which consistently outperformed SAHA. Additional cell survival assays demonstrated that TFPAs were more selective for cancer cells over normal cells. In in vitro HDAC isoform selectivity studies utilizing purified HDAC proteins, the compounds inhibited HDAC enzymes with low nanomolar IC₅₀ values, while displaying some selectivity for HDAC 8. Compound 83 in particular displayed between 11 and 460-fold selectivity for HDAC8 over other isoforms. Western blot analysis revealed the ability of the compounds to inhibit HDAC proteins in a cellular environment as well. Metabolism studies using the S9-fraction assay showed that TFMK 91 undergoes complete metabolic reduction in 15 minutes, whereas TFPA 60 is completely unaffected over 2 hours by the reducing enzymes present in the S9-fraction. Thus, the conversion of TFMK group to TFPA group has resulted in TFMK surrogates as metabolically stable potent HDAC inhibitors which maintain a stable keto-hydrate form resistant to reduction by carbonyl reductases. These compounds constitute a new class of HDAC inhibitors with a metabolically stable metal-binding group that may be used as a template for designing selective HDAC inhibitors by varying the nature of the linker and the cap group.

### Materials and Methods

All chemicals and solvents were purchased from commercial sources and used without further purification, unless stated otherwise. ¹H and ¹³C NMR spectra were recorded on Bruker Avance 600 MHz, INOVA 600 MHz, and Varian VXRS 400 MHz NMR spectrometers in deuterated solvents using residual undeuterated solvents as internal standard. High-resolution mass spectra (HRMS) were recorded on a Waters Synapt high-definition mass spectrometer (HDMS) equipped with nano-ESI source. Melting points were determined using a Fisher-Johns melting point apparatus. Purification of crude products was performed by either flash chromatography on silica gel (40-63 micron) from Sorbent Technologies or on a Teledyne ISCO CombiFlash Companion chromatography system on RediSep prepacked silica cartridges. Thin layer chromatography (TLC) plates (20 cm×20 cm) were purchased from Sorbent Technologies (catalog #4115126) and were viewed under Model UVG-54 mineral light lamp UV-254 nm. A Shimadzu Prominence HPLC with an LC20AT solvent delivery system coupled to a Shimadzu Prominence SPD 20AV Dual wavelength UV/Vis absorbance Detector, a Shimadzu C₁₈ column (1.9 μm, 2.1 mm×50 mm) and HPLC grade solvents (MeOH, H₂O with 0.1% formic acid) were used to determine the purity of compounds by HPLC. All compounds were >95% pure by HPLC analysis.

### General Procedure 1

Preparation of bromoanilides: A mixture of n-bromoalkanoic acid 1a-1d (40 mmol), aniline (4 mL, 44 mmol, 1.1 equiv), EDCl (7.67 g, 40 mmol, 1 equiv), and 4-dimethylaminopyridine (10 mol %, 488 mg) was stirred in anhydrous dichloromethane (35 mL) at room temperature overnight. The mixture was washed with 1M HCl (25 mL), saturated aqueous sodium bicarbonate (25 mL) and brine (25 mL). The organic layer was dried (Na₂SO₄), and the mixture was concentrated in vacuo to give 2a-2d, which was used in the next reaction without further purification.

### 5-Bromo-N-phenylpentanamide (2a)

¹H NMR (600 MHz, CDCl₃) δ 7.52 (d, J=7.8 Hz, 2H), 7.33 (t, J=7.9 Hz, 2H), 7.12 (t, J=7.4 Hz, 1H), 3.46 (t, J=6.5 Hz, 2H), 2.42 (t, J=7.2 Hz, 2H), 2.01-1.95 (m, 2H), 1.94-1.87 (m, 2H).

### 6-Bromo-N-phenylhexanamide (2b)

¹H NMR (600 MHz, CDCl₃) δ 7.48 (d, J=7.9 Hz, 2H), 7.30 (t, J=7.9 Hz, 2H), 7.08 (t, J=7.4 Hz, 1H), 3.40 (t, J=6.7 Hz, 2H), 2.36 (t, J=7.5 Hz, 2H), 1.92-1.85 (m, 2H), 1.78-1.70 (m, 2H), 1.55-1.48 (m, 2H).

### 7-Bromo-N-phenylheptanamide (2c)

¹H NMR (600 MHz, CDCl₃) δ 7.52 (d, J=7.9 Hz, 2H), 7.32 (t, J=7.9 Hz, 2H), 7.11 (t, J=7.4 Hz, 1H), 3.41 (t, J=6.7 Hz, 2H), 2.37 (t, J=7.5 Hz, 2H), 1.91-1.83 (m, 2H), 1.79-1.71 (m, 2H), 1.52-1.45 (m, 2H), 1.44-1.37 (m, 2H).

### 8-Bromo-N-phenyloctanamide (2d)

¹H NMR (600 MHz, CDCl₃) δ 7.52 (d, J=7.9 Hz, 2H), 7.33 (t, J=7.9 Hz, 2H), 7.11 (t, J=7.5 Hz, 1H), 3.42 (t, J=6.8 Hz, 2H), 2.37 (t, J=7.5 Hz, 2H), 1.80-1.69 (m, 2H), 1.49-1.43 (m, 2H), 1.43-1.30 (m, 6H).

### General Procedure 2

Preparation of azidoanilides: A mixture of 2a-2d (5 mmol) and sodium azide (260 mg, 20 mmol, 4 equiv) was heated in N, N-dimethylformamide (5 mL) at 80° C. overnight. The reaction mixture was diluted with ethyl acetate (30 mL) and washed with ice water (5×25 mL). The organic layer was dried (Na₂SO₄) and concentrated in vacuo and the product was used in the next reaction without purification.

### 5-Azido-N-phenylpentanamide (3a)

¹H NMR (600 MHz, CDCl₃) δ 7.52 (d, J=7.9 Hz, 2H), 7.32 (t, J=7.8 Hz, 2H), 7.11 (t, J=7.4 Hz, 1H), 3.32 (t, J=6.7 Hz, 2H), 2.39 (t, J=7.3 Hz, 2H), 1.85-1.77 (m, 2H), 1.71-1.63 (m, 2H).

### 6-Azido-N-phenylhexanamide (3b)

¹H NMR (400 MHz, CDCl₃) δ 7.52 (d, J=7.8 Hz, 2H), 7.32 (t, J=7.9 Hz, 2H), 7.11 (t, J=7.4 Hz, 1H), 3.29 (t, J=6.8 Hz, 2H), 2.38 (t, J=7.4 Hz, 2H), 1.82-1.72 (m, 2H), 1.71-1.59 (m, 2H), 1.53-1.42 (m, 2H).

### 7-Azido-N-phenylheptanamide (3c)

¹H NMR (400 MHz, CDCl₃) δ 7.52 (d, J=7.7 Hz, 2H), 7.33 (t, J=7.9 Hz, 2H), 7.12 (t, J=7.3 Hz, 1H), 3.28 (t, J=6.8 Hz, 2H), 2.38 (t, J=7.4 Hz, 2H), 1.81-1.72 (m, 2H), 1.67-1.61 (m, 2H), 1.49-1.38 (m, 4H).

### 8-Azido-N-phenyloctanamide (3d)

¹H NMR (400 MHz, CDCl₃) δ 7.52 (d, J=7.8 Hz, 2H), 7.33 (t, J=7.8 Hz, 2H), 7.11 (t, J=7.3 Hz, 1H), 3.27 (t, J=6.9 Hz, 2H), 2.37 (t, J=7.5 Hz, 2H), 1.78-1.69 (d, J=6.8 Hz, 2H), 1.67-1.52 (m, 2H), 1.47-1.31 (m, 6H).

### General Procedure 3

To a solution of 3a-3d (4 mmol) in methanol (15 mL) was added 10% palladium on carbon. The suspension was stirred under an atmosphere of H2 (3 atm) overnight. The mixture was filtered through celite and concentrated in vacuo to give 4a-4d as a crude product, which was used in the next reaction without purification.

### 5-Amino-N-phenylpentanamide (4a)

¹H NMR (600 MHz, CDCl₃) δ 7.53 (d, J=8.0 Hz, 2H), 7.33 (t, J=7.9 Hz, 2H), 7.11 (t, J=7.5 Hz, 1H), 2.77 (t, J=6.8 Hz, 2H), 2.41 (t, J=7.4 Hz, 2H), 1.84-2.77 (m, 2H), 1.59-1.52 (m, 2H).

### 6-Amino-N-phenylhexanamide (4b)

¹H NMR (600 MHz, CDCl₃) δ 7.52 (d, J=7.9 Hz, 2H), 7.31 (t, J=7.7 Hz, 2H), 7.10 (t, J=7.3 Hz, 1H), 2.71 (t, J=6.8 Hz, 2H), 2.37 (t, J=7.4 Hz, 2H), 1.79-1.71 (m, 2H), 1.54-1.46 (m, 2H), 1.46-1.39 (m, 2H).

### 7-Amino-N-phenylheptanamide (5c)

¹H NMR (600 MHz, CDCl₃) δ 7.52 (d, J=7.9 Hz, 2H), 7.31 (t, J=7.7 Hz, 2H), 7.10 (t, J=7.3 Hz, 1H), 2.71 (t, J=6.8 Hz, 2H), 2.37 (t, J=7.4 Hz, 2H), 1.79-1.71 (m, 2H), 1.54-1.46 (m, 2H), 1.46-1.39 (m, 2H).

### 8-Amino-N-phenyloctanamide (5d)

¹H NMR (600 MHz, CDCl₃) δ 7.49 (d, J=7.9 Hz, 2H), 7.29 (t, J=7.9 Hz, 2H), 7.07 (t, J=7.4 Hz, 1H), 2.65 (t, J=7.0 Hz, 2H), 2.33 (t, J=7.5 Hz, 2H), 1.75-1.67 (m, 2H), 1.44-1.38 (m, 2H), 1.38-1.28 (m, 6H).

### General Procedure 4—Synthesis of Pyruvate Amides

To a stirred solution of pyruvic acid (1.2 equiv.) in DCM (1 mL/mmol), oxalyl chloride (1.2 equiv.) was added dropwise at 0° C., followed by the addition of catalytic amount of DMF (few drops). Then it was allowed to warm at room temperature and stirred for 3 hours. The resulting mixture was transferred with a glass syringe to a suspension of the n-amino-anilide (1 equiv.) and triethylamine (1.6 equiv.) in DCM (1 mL/mmol) at 0° C., under nitrogen balloon. The reaction was left again to warm at room temperature and stirred overnight. Brine was added to the resulting mixture and was extracted with ethyl acetate. The combined organic layers were dried over anhydrous Na₂SO₄, filtered, and condensed under reduced pressure. The crude mixture was purified by on silica gel chromatography in petroleum ether/ethyl acetate (0->70% ethyl acetate) yielding pure products.

### General Procedure S—Synthesis of Pyruvate Oximes

To a stirred solution of the corresponding pyruvate amides in ethanol (0.04 M) an aqueous solution of the hydroxylamine (2 equiv., 0.4 M concentration) was added, and the reaction was left to stir for 4 hours at room temperature. The resulting mixture was worked up using brine and extracted with ethyl acetate. The combined organic layers were dried using sodium sulfate, filtered, and condensed under reduced pressure resulting in pure oximes. The isomeric mixture of oximes was used without further purification.

### General Procedure 6—Synthesis of Oxamates

To a stirred solution of oxamic acid (1.2 equiv.) in DCM (1 mL/mmol), oxalyl chloride (1.2 equiv.) was added dropwise at 0° C. followed by the addition of catalytic amount of DMF (few drops). Then it was let to warm at room temperature and stirred for 3 hours. The resulting mixture was transferred with a glass syringe to a suspension of the n-amino-anilide (1 equiv.) and triethylamine (1.6 equiv.) in DCM (1 mL/mmol) at 0° C., under nitrogen balloon. The reaction was left again to warm at room temperature and stirred overnight. Brine was added to the resulting mixture and was extracted with ethyl acetate. The combined organic layers were dried over anhydrous Na₂SO₄, filtered, and concentrated in vacuo to yield a white solid which was recrystallized using ethyl acetate/acetone with a few drops of MeOH to yield pure compounds.

### General Procedure 7—Synthesis of Kojic Anilides

A mixture of 5-acetoxy-4-oxo-4H-pyran-2-carboxylic acid 94 (0.25 mmol, 1 equiv), dimethylformamide (10 μL), and oxalyl chloride (150 μL of 2.0 M in dichloromethane, 0.3 mmol, 1.2 equiv) in anhydrous dichloromethane (1 mL) under an atmosphere of N₂ was stirred for four hours at room temperature. A solution of aminoalkanamide (0.26 mmol, 1.1 equiv) in anhydrous dichloromethane (500 μL) was added over ten minutes and the mixture was stirred overnight at room temperature. Solvent was removed under reduced pressure and the residue was dissolved in ethyl acetate (5 mL), washed with brine (3×1 mL), and the solvent was removed under reduced pressure. The crude solid product was dissolved in anhydrous methanol, sodium methoxide (16 mg, 0.3 mmol, 1.2 equiv) was added, and the reaction was continued until completion as monitored by TLC. The solvent was removed under reduced pressure and the residue was dissolved in water (0.5 mL) and washed with ethyl acetate (0.5 mL) three times. The aqueous layer was acidified with 1M HCl (1 mL), and extracted with ethyl acetate (3×1 mL). The organic extract was dried over Na₂SO₄, concentrated in vacuo, and purified by flash chromatography (methanol/DCM 2-4%) to give compounds 9a-9d.

### 5-(2-oxopropanamido)-N-phenylpentanamide (5a)

Synthesized according to general procedure 4. Yield 33%. ¹H NMR (600 MHz, CDCl₃) δ 7.54 (d, J=7.9 Hz, 2H), 7.43 (s, 1H(NH)), 7.33 (t, J=7.9 Hz, 2H), 7.11 (d, 1H-s, 1H(NH) overlapping), 3.37 (q, J=6.7 Hz, 2H), 2.49 (s, 3H), 2.42 (t, J=7.4 Hz, 2H), 1.81-1.74 (m, 2H), 1.66 (dt, J=14.0, 6.9 Hz, 2H). ¹³C NMR (151 MHz, CDCl₃) δ 197, 170.8, 160.4, 129, 124.2, 119.6, 38.5, 36.7, 28.7, 24.5, 22.5.

### 6-(2-oxopropanamido)-N-phenylhexanamide (5b)

Synthesized according to general procedure 4. Yield 36%. ¹H NMR (600 MHz, CDCl₃) δ 7.53 (t, J=9.4 Hz, 2H), 7.40 (s, 1H), 7.32 (t, J=7.8 Hz, 2H), 7.10 (t, J=7.4 Hz, 1H), 7.03 (s, 1H), 3.31 (dd, J=13.6, 6.8 Hz, 2H), 2.47 (s, 3H), 2.37 (t, J=7.4 Hz, 2H), 1.76 (dt, J=15.1, 7.5 Hz, 2H), 1.63-1.55 (m, 2H), 1.46-1.37 (m, 2H). ¹³C NMR (151 MHz, MeOD) δ 196.3, 173, 172.3, 161.4, 138.4, 128.5, 123.9, 119.8, 38.5, 36.7, 29.2, 28.2, 26.5, 24.9, 24.2, 23.

### 7-(2-oxopropanamido)-N-phenylheptanamide (5c)

Synthesized according to general procedure 4. Yield 26%. ¹H NMR (600 MHz, CDCl₃) δ 7.53 (d, J=8.0 Hz, 2H), 7.32 (dd, J=15.7, 7.8 Hz, 2H), 7.24 (s, 1H), 7.10 (dd, J=18.9, 11.5 Hz, 1H), 6.97 (s, 1H), 3.30 (dd, J=13.4, 6.8 Hz, 2H), 2.49 (s, 3H), 2.36 (t, J=7.4 Hz, 2H), 1.79-1.71 (m, 2H), 1.57 (dt, J=14.0, 6.9 Hz, 2H), 1.41 (dd, J=26.1, 12.5 Hz, 4H). ¹³C NMR (151 MHz, CDCl₃) δ 197.34, 171.1, 160.1, 138.0, 128.9, 124.1, 119.6, 39.5, 37.7, 29.03, 28.6, 26.5, 25.3, 24.2. HRMS calcd for C₁₆H₂₃N₂O₃ (M+H) 291.1709 found 291.1693.

### 5-(2-(hydroxyimino)propanamido)-N-phenylpentanamide (6a)

Synthesized according to general procedure 5. Yield 90%, ¹H NMR (600 MHz, CD₃OD) δ 7.55 (d, J=7.6 Hz, 2H), 7.31 (t, J=8.0 Hz, 2H), 7.10 (t, J=7.4 Hz, 1H), 2.42 (t, J=7.4 Hz, 2H), 2.05 (s, 2H), 1.98 (s, 3H), 1.77-1.71 (m, 2H), 1.66-1.59 (m, 2H). ¹³C NMR (151 MHz, MeOD) δ 173.1, 165.3, 150.3, 138.6, 128.2, 123.6, 119.6, 38.5, 35.8, 29.1, 22.6, 8.3. HRMS calcd for C₁₄H₂₀N₃O₃ (M+H) 278.1505 found 278.1517.

### 6-(2-(hydroxyimino)propanamido)-N-phenylhexanamide (6b)

Synthesized according to general procedure 5. Yield 91%. ¹H NMR (600 MHz, CD₃OD) δ 7.50 (d, J=5.3, 3.3 Hz, 2H), 7.26 (t, J=10.7, 5.2 Hz, 2H), 7.04 (t, J=7.4 Hz, 1H), 3.23 (t, J=7.1 Hz, 2H), 2.34 (t, J=7.5 Hz, 2H), 1.92 (s, 3H), 1.75-1.62 (m, 2H), 1.60-1.48 (m, 2H), 1.43-1.32 (m, 2H). ¹³C NMR (151 MHz, MeOD) δ 173.1, 165.1, 150.1, 138.5, 128.5, 124.2, 120.3, 38.9, 36.7, 28.9, 26.2, 25.1, 7.7.

### 7-(2-(hydroxyimino)propanamido)-N-phenylheptanamide (6c)

Synthesized according to general procedure 5. Yield 94%. ¹H NMR (600 MHz, CD₃OD) S 7.51 (d, 2H), 7.26 (t, J=7.9 Hz, 2H), 7.04 (t, J=7.4 Hz, 1H), 3.21 (t, J=7.1 Hz, 2H), 2.33 (t, J=7.5 Hz, 2H), 1.93 (s, 3H), 1.70-1.63 (m, 2H), 1.55-1.48 (m, 2H), 1.42-1.31 (m, 4H). ¹³C NMR (151 MHz, CD₃OD) δ 173.1, 164.9, 150.1, 138.6, 128.2, 123.5, 120.1, 38.6, 36.5, 29, 28.4, 26.3, 25.4, 7.9.

### N1-(5-oxo-5-(phenylamino)pentyl)oxalamide (7a)

Synthesized according to general procedure 6. Yield 24%. ¹H NMR (600 MHz, DMSO) δ 9.87 (s, 1H), 8.74 (t, J=5.9 Hz, 1H), 8.04 (s, 1H), 7.77 (s, 1H), 7.58 (d, J=7.6 Hz, 2H), 7.28 (t, J=7.9 Hz, 2H), 7.02 (t, J=7.4 Hz, 1H), 3.14 (dd, J=13.3, 6.7 Hz, 2H), 2.31 (t, J=7.3 Hz, 2H), 1.60-1.45 (m, 4H). ¹³C NMR (151 MHz, DMSO) δ 171.5, 162.7, 160.7, 139.7, 129.2, 123.3, 119.4, 39, 36.5, 28.9, 22.9.

### N1-(6-oxo-6-(phenylamino)hexyl)oxalamide (7b)

Synthesized according to general procedure 6. Yield 21%. ¹H NMR (600 MHz, DMSO) δ 9.86 (s, 1H), 8.70 (t, J=6.0 Hz, 1H), 8.04 (s, 1H), 7.76 (s, 1H), 7.59 (d, J=7.6 Hz, 2H), 7.28 (t, J=7.9 Hz, 2H), 7.02 (t, J=7.4 Hz, 1H), 3.11 (dd, J=13.5, 6.8 Hz, 2H), 2.29 (t, J=7.5 Hz, 2H), 1.64-1.54 (m, 2H), 1.53-1.44 (m, 2H), 1.32-1.23 (m, 2H). ¹³C NMR (151 MHz, DMSO) δ 171.6, 162.8, 160.6, 139.8, 129.1, 123.4, 119.5, 39.2, 36.8, 29, 26.5, 25.3.

### N1-(8-oxo-8-(phenylamino)octyl)oxalamide (7d)

Synthesized according to general procedure 6. Yield 35%. ¹H NMR (600 MHz, DMSO) δ 9.85 (s, 1H), 8.68 (t, J=6.0 Hz, 1H), 8.03 (s, 1H), 7.76 (s, 1H), 7.59 (d, J=7.7 Hz, 2H), 7.28 (t, J=7.9 Hz, 2H), 7.02 (t, J=7.4 Hz, 1H), 3.10 (dd, J=13.5, 6.8 Hz, 2H), 2.29 (t, J=7.5 Hz, 2H), 1.60-1.54 (m, 2H), 1.49-1.40 (m, 2H), 1.33-1.19 (m, 6H). ¹³C NMR (151 MHz, DMSO) δ 171.7, 162.9, 160.6, 139.8, 129, 123.6, 119.1, 39.2, 36.9, 29.2, 29.1, 29, 26.7, 25.6.

### N-(6-oxo-6-(phenylamino)hexyl)-1H-pyrrole-2-carboxamide (8b)

To a stirred suspension of 1H-pyrrole-2-carboxylic acid (230 mg, 2.1 mmol) in DCM (8 mL), were added, EDC-HCl (600 mg, 3.1 mmol, 1.5 equiv.), cat. amount of DMAP (approximately 10 mol %), and 6-amino-N-phenylhexanamide 4b (428 mg, 2.1 mmol, 1 equiv.). The resulting mixture was stirred at room temperature overnight, after which it was diluted with brine and ethyl acetate. The organic layer was separated, dried over Na₂SO₄, filtered, and concentrated in vacuo. The crude mixture was separated on silica gel chromatography in ethyl acetate/hexanes to provide N-(6-oxo-6-(phenylamino) hexyl)-1H-pyrrole-2-carboxamide (8b) (403 mg, 64% yield). ¹H NMR (600 MHz, CD₃CN) δ 9.93 (s, 1H), 8.32 (s, 1H), 7.57 (d, J=8.0 Hz, 2H), 7.32 (t, J=7.9 Hz, 2H), 7.09 (t, J=7.4 Hz, 1H), 6.90 (d, J=1.1 Hz, 1H), 6.79 (s, 1H), 6.65 (s, 1H), 6.17 (dt, J=5.3, 1.6 Hz, 1H), 3.33 (dd, J=13.2, 6.7 Hz, 2H), 2.34 (t, J=7.4 Hz, 2H), 1.97 (dt, J=4.8, 2.4 Hz, 2H), 1.74-1.66 (m, 2H), 1.63-1.56 (m, 2H), 1.45-1.38 (m, 2H). ¹³C NMR (151 MHz, CD₃CN) δ 171.6, 160.8, 139.1, 128.8, 126.5, 123.4, 120.9, 119.5, 109.2, 108.81, 38.5, 36.6, 29.2, 26.2, 24.9. HRMS calcd for C₁₇H₂₂N₃O₂ (M+H) 264.1348 found 264.1354.

### 5-Hydroxy-4-oxo-N-(5-oxo-5-(phenylamino)pentyl)-4H-pyran-2-carboxamide (9a)

Synthesized according to general procedure 7. 12.75 mg (16%). ¹H NMR (400 MHz, CD₃OD) δ 8.00 (s, 1H), 7.53 (d, J=7.7 Hz, 2H), 7.29 (t, J=7.9 Hz, 2H), 7.08 (d, J=7.3 Hz, 1H), 7.05 (s, 1H), 3.43-3.38 (m, 2H), 2.42 (t, J=7.2 Hz, 2H), 1.80-1.64 (m, 4H). ¹³C NMR (151 MHz, CD₃OD) S 175, 172.8, 159.4, 155.8, 147.7, 139.2, 138.5, 128.4, 123.7, 119.8, 112.8, 39.2, 39.1, 36, 28.4, 22.7. HRMS-ESI M/z: [M+H]⁺ calcd. 331.1293 for C₁₇H₁₈N₂O₅. found 331.1310.

### 5-Hydroxy-4-oxo-N-(6-oxo-6-(phenylamino)hexyl)-4H-pyran-2-carboxamide (9b)

Synthesized according to general procedure 7.14 mg (17.6%). ¹H NMR (400 MHz, CD₃OD) δ 7.99 (s, 1H), 7.51 (d, J=7.6 Hz, 2H), 7.28 (d, J=7.5 Hz, 2H), 7.06 (t, J=7.4 Hz, 1H), 7.02 (s, 1H), 3.38 (q, J=6.8 Hz, 2H), 2.38 (t, J=7.4 Hz, 2H), 1.79-1.70 (m, 2H), 1.70-1.59 (m, 2H), 1.50-1.35 (m, 2H). ¹³C NMR (151 MHz, CD₃OD) δ 175, 173.1, 159.4, 155.8, 147.7, 139.2, 138.5, 128.4, 123.7, 119.8, 112.7, 39.4, 39.2, 36.3, 28.6, 26, 25.1. HRMS-ESI M/z: [M+H]⁺ calcd. 345.145 for C₁₈H₂₀N₂O₅. found 345.1437.

### 5-Hydroxy-4-oxo-N-(7-oxo-7-(phenylamino)heptyl)-4H-pyran-2-carboxamide (9c)

Synthesized according to general procedure 7. 12.4 mg (14%). ¹H NMR (600 MHz, CD₃OD) δ 8.05 (s, 1H), 7.55 (d, J=7.9 Hz, 2H), 7.30 (t, J=7.8 Hz, 2H), 7.09 (t, J=7.4 Hz, 1H), 7.07 (s, 1H), 3.41-3.38 (m, 2H), 2.40 (t, J=7.4 Hz, 2H), 1.78-1.69 (m, 2H), 1.68-1.61 (m, 2H), 1.49-1.40 (m, 4H). ¹³C NMR (151 MHz, CD₃OD): δ 175, 173.2, 159.4, 159.3, 155.9, 155.9, 147.6, 139.3, 138.5, 128.4, 123.7, 119.8, 112.8, 39.5, 39.4, 36.4, 28.7, 28.5, 26.3, 25.4. HRMS-ESI M/z: [M+H]⁺ calcd. 359.1606 for C₁₉H₂₂N₂O₅. found 359.1618.

### 5-Hydroxy-4-oxo-N-(8-oxo-8-(phenylamino)octyl)-4H-pyran-2-carboxamide (9d)

Synthesized according to general procedure 7. 20 mg (6%). ¹H NMR (600 MHz, CD₃OD) S 8.01 (s, 1H), 7.55 (dd, J=8.5, 0.9 Hz, 2H), 7.31 (t, J=7.2 Hz, 2H), 7.09 (t, J=7.4 Hz, 1H), 7.05 (s, 1H), 3.37 (t, J=7.2 Hz, 2H), 2.38 (t, J=7.5 Hz, 2H), 1.76-1.69 (m, 2H), 1.66-1.59 (m, 2H), 1.4-1.36 (m, 6H). ¹³C NMR (151 MHz, CD₃OD): δ 175.4, 159.3, 155.7, 139.2, 138.5, 128.4, 123.7, 119.8, 112.7, 39.4, 36.5, 28.8, 28.6, 26.4, 25.4. HRMS-ESI M/z: [M+H]⁺ calcd. 373.1763 for C₂₀H₂₄N₂O₅. found 373.1767.

### Synthesis of 4-((tert-butoxycarbonyl)amino)butanoic acid (12)

In a 250 mL round-bottom flask equipped with a stir-bar, 4-aminobutanoic acid (11) (5 g, 48.5 mmol, 1 equiv) was first dissolved in a mixture of acetone (50 mL) and water (50 mL). Triethylamine (13.6 mL, 97 mmol, 2 equiv) was added to the reaction mixture, followed by di-tert-butyl dicarbonate (10.92 g, 75 mmol, 1.03 equiv). The mixture was stirred at room temperature for four hours. Acetone was removed under reduced pressure and the residual water was acidified with 1M HCl (200 mL) and extracted with ethyl acetate (3×100 mL). The organic extract was dried (Na₂SO₄), concentrated in vacuo and triturated in hexanes to yield 4-((tert-butoxycarbonyl) amino) butanoic acid (12) as a white solid (9.23 g, 94%).

### Synthesis of tert-butyl(4-oxo-4-(phenylamino)butyl)carbamate (13)

In a 10 mL round—bottomed flask, a mixture of 4-((tert-butoxycarbonyl) amino) butanoic acid (2 mmol, 490 mg), aniline (2 mmol, 182 μL), EDCl (2 mmol, 384 mg, 1 equiv), DMAP (10 mol %, 24 mg) was stirred in dichloromethane (4 mL) under an atmosphere of nitrogen (balloon) overnight. Solvent was removed under reduced pressure, and the reaction mixture was diluted in ethyl acetate (10 mL) and washed with 1 M HCl (10 mL), saturated NaHCO₃(10 mL) (aq.) and brine (10 mL). The organic layer was dried (Na₂SO₄), concentrated in vacuo, and purified using flash chromatography (ethyl acetate/hexanes 10-70%) to yield tert-butyl (4-oxo-4-(phenylamino) butyl) carbamate as a white solid, 450 mg, 81%. ¹H NMR (600 MHz, CDCl₃) δ 7.62 (d, J=7.7 Hz, 2H), 7.33 (t, J=7.9 Hz, 2H), 7.10 (t, J=7.3 Hz, 1H), 3.30-3.22 (m, 2H), 2.46-2.34 (m, 2H), 1.95-1.84 (m, 2H), 1.47 (s, 9H).

### Synthesis of 7-((tert-butoxycarbonyl)amino)heptanoic acid (16)

In a 250 mL round—bottom flask equipped with a stir-bar, 7-aminoheptanoic acid (15) (12.72 g, 70 mmol, 1 equiv) was first dissolved in a mixture of acetone (50 mL) and water (50 mL). Triethylamine (19.53 mL, 140 mmol, 2 equiv) was added to the reaction mixture, followed by di-tert-butyl dicarbonate (16.37 g, 75 mmol, 1.07 equiv). The mixture was stirred at room temperature for four hours. Acetone was first removed under reduced pressure and the residual water was acidified with 1M HCl (200 mL) and extracted with ethyl acetate (100 mL) three times. The organic extract was dried (Na₂SO₄), concentrated in vacuo, and triturated in hexanes to yield 7-((tert-butoxycarbonyl) amino) heptanoic acid (16) as a white solid (15.7 g, 91%).

### Synthesis of Boc-arenes

In a 10 mL round-bottomed flask, a mixture of 7-((tert-butoxycarbonyl) amino) heptanoic acid (2 mmol, 490 mg), the respective aminoarene (2 mmol), EDCl (2 mmol, 384 mg, 1 equiv), DMAP (10 mol %, 24 mg) was stirred in dichloromethane (4 mL) under an atmosphere of nitrogen (balloon) overnight. Solvent was removed under reduced pressure, and the reaction mixture was diluted in ethyl acetate (10 mL) and washed with 1 M HCl (10 mL), saturated NaHCO₃(10 mL) (aq.) and brine (10 mL). The organic layer was dried (Na₂SO₄), concentrated in vacuo, and purified using flash chromatography (ethyl acetate/hexanes 10-70%) to yield Boc-arenes 17-50.

An alternative procedure involved using a mixture of 7-((tert-butoxycarbonyl) amino) heptanoic acid (2 mmol, 490 mg), the aminoarene (2 mmol), HATU (2 mmol, 760 mg, 1 equiv), HOBt (1 mmol, 135 mg, 1 equiv), and NMM (660 μl, 6 mmol, 3 equiv) in dimethylformamide (4 mL) under an atmosphere of nitrogen (balloon) overnight. The reaction mixture was diluted in ethyl acetate (10 mL), washed with ice water (5×10 mL) and dried (Na₂SO₄). The organic layer was concentrated in vacuo and purified in the same manner as above.

### Tert-butyl(7-((4-acetylphenyl)amino)-7-oxoheptyl)carbamate (17)

¹H NMR (400 MHz, CDCl₃) δ 7.94 (d, J=8.7 Hz, 2H), 7.69 (d, J=8.3 Hz, 2H), 3.14 (q, J=6.1 Hz, 2H), 2.39 (t, 2H), 1.80-1.71 (m, 2H), 1.54-1.48 (m, 2H), 1.46 (s, 9H), 1.43-1.34 (m, 4H). Obtained a partially purified product that was used in the next step.

### Tert-butyl(7-((4-methoxyphenyl) amino)-7-oxoheptyl)carbamate (18)

White solid, 200 mg (29%) mp. 120-125° C. ¹H NMR (400 MHz, CDCl₃) δ 7.44 (d, J=8.5 Hz, 2H), 6.86 (d, J=8.5 Hz, 2H), 3.80 (s, 3H), 3.13 (q, J=5.1 Hz, 2H), 2.33 (t, J=7.3 Hz, 2H), 1.80-1.70 (m, 2H), 1.54-1.47 (m, 2H), 1.45 (s, 9H), 1.41-1.31 (m, 4H). ¹³CNMR (151 MHz, CDCl₃): 171.3, 156.3, 156.2, 131.2, 121.7, 114.1, 79.2, 55.5, 40.2, 37.3, 29.9, 28.5, 28.5, 26.1, 25.5.

### Tert-butyl(7-((4-fluorophenyl) amino)-7-oxoheptyl)carbamate (19)

White solid, 145 mg (21.4%) mp. 90-95° C. ¹H NMR (400 MHz, CDCl₃) δ 7.44 (d, J=8.6 Hz, 2H), 6.86 (d, J=8.9 Hz, 2H), 3.17-3.06 (m, 2H), 2.33 (t, J=7.3 Hz, 2H), 1.79-1.68 (m, 2H), 1.54-1.48 (m, 2H), 1.45 (s, 9H), 1.41-1.31 (m, 2H). ¹³CNMR (151 MHz, CDCl₃): 171.6, 160, 158.4, 156.3, 134.2, 121.6, 121.5, 115.6, 115.5, 79.3, 40.1, 37.2, 29.9, 28.5, 28.3, 26, 25.4.

### Tert-butyl (7-oxo-7-(pyridin-4-ylamino) heptyl)carbamate (20)

¹H NMR (400 MHz, CDCl₃) δ 8.48 (d, J=5.4 Hz, 2H), 7.61 (d, J=4.6 Hz, 2H), 3.15 (q, 6.5 Hz, 2H), 2.40 (t, J=6.9 Hz, 2H), 1.80-1.70 (m, 2H), 1.53-1.49 (m, 2H), 1.47 (s, 9H), 1.43-1.34 (m, 4H). Obtained a partially purified product, which was used in the next step. Also, it was slow to purification until 4-7% methanol/DCM was used.

### Tert-butyl (7-oxo-7-((4-(pentafluoro-λ⁶-sulfaneyl)phenyl) amino)heptyl)carbamate (21)

¹H NMR (400 MHz, CDCl₃) δ 7.69 (s, 4H), 3.13 (q, J=5.7 Hz, 2H), 2.37 (t, J=7.1 Hz, 2H), 1.79-1.70 (m, 2H), 1.53-1.48 (m, 2H), 1.45 (s, 9H), 1.41-1.35 (m, 4H). Obtained a partially purified product, which was used in the next step.

### Tert-butyl (7-((2,4-difluorophenyl) amino)-7-oxoheptyl)carbamate (22)

White solid, 300 mg (42%) mp. 75-80° C. ¹H NMR (400 MHz, CDCl₃) δ 8.26 (dd, J=15.3, 9.0 Hz, 1H), 6.93-6.79 (m, 2H), 3.12 (q, J=5.8 Hz, 2H), 2.40 (t, J=7.5 Hz, 2H), 1.80-1.68 (m, 2H), 1.54-1.48 (m, 2H), 1.45 (s, 9H), 1.42-1.32 (m, 4H). ¹³CNMR (151 MHz, CDCl₃): 171.6, 160, 158.4, 156.3, 134.2, 121.6 (d, J=7.8 Hz), 115.6 (d, J=22.4 Hz), 79.3, 40.1, 37.2, 29.9, 28.3, 26, 25.4.

### Tert-butyl (7-oxo-7-((2,4,6-trifluorophenyl) amino) heptyl)carbamate (23)

¹H NMR (400 MHz, CDCl₃) δ 6.73 (t, J=8.1 Hz, 2H), 3.12 (q, J=7.0 Hz, 2H), 2.34 (t, J=7.5 Hz, 2H), 1.92-1.81 (m, 2H), 1.57-1.48 (m, 2H), 1.44 (s, 9H), 1.40-1.30 (m, 4H). Obtained a crude product which was used in the next step without purification.

### Tert-butyl (7-oxo-7-((5-phenylthiazol-2-yl) amino) heptyl)carbamate (24)

¹H NMR (600 MHz, CDCl₃) δ 7.79 (d, J=7.2 Hz, 2H), 7.44 (t, J=7.6 Hz, 2H), 7.37 (t, J=7.4 Hz, 1H), 7.13 (s, 1H), 3.16-3.07 (m, 2H), 2.28 (t, J=7.5 Hz, 2H), 1.71-1.59 (m, 2H), 1.47 (s, 9H), 1.43-1.30 (m, 4H). Obtained a partially purified product that was used in the next step.

### Tert-butyl (7-oxo-7-((3,4,5-trimethoxyphenyl) amino) heptyl)carbamate (25)

¹HNMR (400 MHz, CDCl₃): δ 6.91 (s, 2H), 3.15 (d, J=6 Hz, 2H), 2.36 (t, J=7.2 Hz, 2H), 1.77-1.69 (m, 2H), 1.51-1.45 (m, 2H), 1.44 (s, 9H), 1.4-1.35 (m, 4H). ¹³CNMR (151 MHz, CDCl₃): δ 171.6, 153.3, 134.4, 134.3, 97.3, 79.3, 61, 56.1, 40.12, 37.34, 30, 28.5, 28.3, 29.6, 25.4.

### Tert-butyl (7-(naphthalen-1-ylamino)-7-oxoheptyl)carbamate (26)

Pink solid, 778 mg, quantitative yield, mp. 90-95° C. ¹H NMR (600 MHz, CDCl₃) δ 7.92-7.86 (m, 3H), 7.71 (d, J=8.0 Hz, 1H), 7.54-7.50 (m, 2H), 7.47 (t, J=7.7 Hz, 1H), 3.14 (d, J=5.4 Hz, 2H), 2.51 (t, J=7.2 Hz, 2H), 1.85-1.77 (m, 2H), 1.55-1.49 (m, 2H), 1.46 (s, 9H), 1.41-1.31 (m, 4H). ¹³C NMR (151 MHz, CDCl₃): 172, 156.1, 134.1, 132.4, 128.7, 128.5, 127.3, 126.2, 126, 125.8, 125.7, 121.3, 120.9, 79.1, 40.4, 37.4, 30, 28.8, 28.5, 26.4, 25.7.

### Tert-butyl (7-((4-benzoylphenyl)amino)-7-oxoheptyl)carbamate (27)

¹H NMR (600 MHz, CDCl₃) δ 7.81 (d, J=8.6 Hz, 2H), 7.77 (t, J=8.3 Hz, 4H), 7.59 (t, J=7.4 Hz, 1H), 7.48 (t, J=7.7 Hz, 2H), 3.11 (d, J=6.0 Hz, 2H), 2.38 (t, J=7.2 Hz, 2H), 1.75-1.68 (m, 2H), 1.50-1.47 (m, 2H), 1.46 (s, 9H), 1.38-1.29 (m, 4H). ¹³C NMR (151 MHz, CDCl₃) δ 196, 172.3, 156.3, 142.7, 137.9, 132.5, 132.3, 131.6, 130.1, 129.9, 128.3, 128.1, 118.8, 79.3, 40.2, 37.3, 30, 28.5, 28.4, 26.1, 25.4.

### Tert-butyl (7-((2,5-dimethoxyphenyl amino)-7-oxoheptyl)carbamate (28)

Brown solid, 500 mg (65.7%) mp. 80-85° C. ¹H NMR (400 MHz, CDCl₃) δ 8.15 (d, J=2.5 Hz, 1H), 6.79 (d, J=8.9 Hz, 1H), 6.57 (dd, J=8.9, 2.9 Hz, 1H), 3.85 (s, 3H), 3.79 (s, 3H), 3.12 (d, J=6.3 Hz, 2H), 2.40 (t, J=7.4 Hz, 2H), 1.78-1.70 (m, 2H), 1.54-1.47 (m, 2H), 1.45 (s, 9H), 1.42-1.32 (m, 4H). ¹³C NMR (151 MHz, CDCl₃) δ 171.2, 153.9, 141.8, 128.4, 110.6, 108.5, 105.7, 56.2, 55.8, 40.5, 37.9, 29.9, 28.9, 28.4, 26.6, 25.4.

### Tert-butyl (7-((4-((4-methylphenyl)sulfonamido)phenyl) amino)-7-oxoheptyl)carbamate (29)

White solid, 588 mg (60.1%) mp. 140-145° C. ¹H NMR (600 MHz, CDCl₃) δ 7.62 (d, J=7.9 Hz, 2H), 7.46 (d, J=8.4 Hz, 2H), 7.24 (d, J=8.0 Hz, 2H), 7.02 (d, J=8.4 Hz, 2H), 3.13 (d, J=5.8 Hz, 2H), 2.40 (s, 3H), 2.34 (t, J=7.2 Hz, 2H), 1.76-1.69 (m, 2H), 1.52-1.48 (m, 2H), 1.47 (s, 9H), 1.43-1.33 (m, 4H). ¹³C NMR (151 MHz, CDCl₃): δ 171.5, 156.2, 143.9, 135.9, 132, 129.7, 127.3, 123.5, 120.5, 40.1, 37.3, 30, 28.5, 28.3, 25.9, 25.4, 21.6.

### Tert-butyl (7-((2-methoxyphenyl)amino)-7-oxoheptyl)carbamate (30)

¹H NMR (600 MHz, CDCl₃) δ 8.41 (dd, J=8.0, 1.3 Hz, 1H), 7.05 (td, J=7.9, 1.5 Hz, 1H), 6.98 (td, J=7.8, 1.2 Hz, 1H), 6.90 (dd, J=8.1, 1.1 Hz, 1H), 3.91 (s, 3H), 3.14 (q, J=6.2 Hz, 2H), 2.41 (t, J=7.5 Hz, 2H), 1.80-1.72 (m, 2H), 1.55-1.48 (m, 2H), 1.46 (s, 9H), 1.44-1.36 (m, 4H). ¹³C NMR (151 MHz, CDCl₃): δ 171.1, 156, 147.7, 127.7, 123.5, 121.1, 119.7, 109.8, 79, 55.64, 40.5, 37.9, 29.9, 28.9, 28.4, 26.5, 25.5.

### Tert-butyl (7-((4-chlorophenyl) amino)-7-oxoheptyl)carbamate (31)

White solid, 597 mg (8%) mp. 105-110° C. ¹H NMR (600 MHz, CDCl₃) δ 7.54 (d, J=8.4 Hz, 2H), 7.30 (d, J=8.9 Hz, 2H), 3.15 (q, J=6.3 Hz, 2H), 2.36 (t, J=7.3 Hz, 2H), 1.79-1.72 (m, 2H), 1.54-1.49 (m, 2H), 1.47 (s, 9H), 1.44-1.34 (m, 4H). ¹³C NMR (151 MHz, CDCl₃): 171.6, 156.3, 136.8, 129, 121, 79.3, 40, 37.3, 30, 28.5, 28.5, 28.22, 25.9, 25.4.

### Tert-butyl (7-((4-bromophenyl)amino)-7-oxoheptyl)carbamate (32)

White solid, 552 mg (69%) mp. 115-120° C. ¹H NMR (600 MHz, CDCl₃) δ 7.49 (d, J=8.4 Hz, 2H), 7.44 (d, J=8.8 Hz, 2H), 3.15 (q, J=6.1 Hz, 2H), 2.36 (t, J=7.3 Hz, 2H), 1.80-1.73 (m, 2H), 1.53-1.48 (m, 2H), 1.47 (s, 9H), 1.45-1.35 (m, 4H). ¹³C NMR (151 MHz, CDCl₃): δ 171.59, 156.2, 137.3, 131.9, 121.3, 116.5, 79.3, 40.1, 37.3, 30, 28.5, 28.3, 25.9, 25.4.

### Tert-butyl (7-([1,1′-biphenyl]-4-ylamino)-7-oxoheptyl)carbamate (33)

White solid, 500 mg, (63%) mp. 150-155° C. ¹H NMR (400 MHz, CDCl₃) δ 7.64 (d, J=8.4 Hz, 2H), 7.60-7.54 (m, 4H), 7.43 (t, J=7.6 Hz, 2H), 7.33 (t, J=7.3 Hz, 1H), 3.14 (q, J=6.4 Hz, 2H), 2.38 (t, J=7.3 Hz, 2H), 1.82-1.72 (m, 2H), 1.54-1.48 (m, 2H), 1.46 (s, 9H), 1.43-1.35 (m, 4H). ¹³C NMR (151 MHz, CDCl₃): 171.5, 156.2, 140.6, 137.5, 136.9, 128.8, 127.6, 127.1, 126.9, 120, 79.3, 40.2, 37.4, 30, 28.6, 28.6, 26.13, 25.5.

### Tert-butyl (7-oxo-7-(thiazol-2-ylamino) heptyl)carbamate (34)

White solid, 512 mg (78%) mp. 85-90° C. ¹H NMR (400 MHz, CDCl₃) δ 7.43 (d, J=3.6 Hz, 1H), 7.01 (d, J=3.6 Hz, 1H), 3.11 (d, J=6.2 Hz, 2H), 2.55 (t, J=7.4 Hz, 2H), 1.84-1.73 (m, 2H), 1.54-1.47 (m, 2H), 1.44 (s, 9H), 1.40-1.31 (m, 4H). ¹³C NMR (151 MHz, CDCl₃): δ 171.2, 160, 156, 136, 113.5, 79.1, 40.4, 36.1, 29.9, 28.9, 28.5, 26.5, 25.

### Tert-butyl (7-(benzo[d]thiazol-2-ylamino)-7-oxoheptyl)carbamate (35)

¹H NMR (400 MHz, CDCl₃) δ 7.82 (d, J=7.8 Hz, 1H), 7.71 (d, J=8.1 Hz, 1H), 7.44 (t, J=7.7 Hz, 1H), 7.32 (t, J=7.5 Hz, 1H), 3.17-3.04 (m, 2H), 2.52 (t, J=7.4 Hz, 2H), 1.79-1.67 (m, 2H), 1.56-1.46 (m, 2H), 1.44 (s, 9H), 1.41-1.26 (m, 4H). Obtained a partially purified product that was used in the next step.

### Tert-butyl (7-((3-methoxyphenyl) amino)-7-oxoheptyl)carbamate (36)

White solid, 520 mg (74%) mp. 80-85° C. ¹H NMR (400 MHz, CDCl₃): δ 7.35 (s, 1H), 7.16 (t, J=7.8 Hz, 1H), 7.03 (t, J=7.8 Hz, 1H), 6.62 (d, J=8.1 Hz, 1H), 3.74 (s, 3H), 3.06 (q, J=6.3 Hz, 2H), 2.3 (t, J=7.2 Hz, 2H), 1.78-1.68 (m, 2H), 1.43 (s, 9H), 1.4-1.26 (m, 4H). ¹³C NMR (151 MHz, CDCl₃): δ 171.6. 160.1, 156.2, 139.4, 129.6, 11.8, 110, 105.3, 79.2, 40.2, 37.5, 30, 28.5, 26.2, 25.4.

### Tert-butyl (7-((4-methyl-5-phenylthiazol-2-yl)amino)-7-oxoheptyl)carbamate (37)

¹H NMR (400 MHz, CDCl₃) δ 7.56 (d, J=7.3 Hz, 2H), 7.43 (t, J=7.5 Hz, 2H), 7.35 (t, J=7.3 Hz, 1H), 3.09 (q, J=5.6 Hz, 2H), 2.49 (s, CH₃), 2.15 (t, J=6.7 Hz, 2H), 1.66-1.53 (m, 2H), 1.44 (s, 9H), 1.31-1.19 (m, 4H). ¹³C NMR (151 MHz, CDCl₃): δ 171.6, 156.4, 144.2, 135, 129.6, 128.5, 128.4, 128.38, 127.7, 121.8, 79.1, 40.5, 35, 29.8, 28.6, 28.5, 26.4, 24.5, 12.1.

### Tert-butyl (7-((4,5-diphenylthiazol-2-yl) amino)-7-oxoheptyl)carbamate (38)

¹H NMR (400 MHz, CDCl₃) δ 7.42 (dd, J=6.6, 3.0 Hz, 1H), 7.31 (m, 4H), 3.12 (d, J=6.0 Hz, 2H), 2.41-2.30 (m, 2H), 1.76-1.61 (m, 4H), 1.45 (s, 9H), 1.40-1.22 (m, 4H). ¹³C NMR (151 MHz, CDCl₃): δ 171.8, 158, 156, 143.7, 131.9, 129.5, 129.4, 129.1, 128.8, 128.7, 128.6, 128, 127.8, 126.7, 79.1, 40.5, 35, 29.8, 28.6, 28.5, 26.4, 24.5.

### Tert-butyl (7-((2-bromophenyl)amino)-7-oxoheptyl)carbamate (39)

¹H NMR (600 MHz, CDCl₃) δ 8.36 (d, J=7.0 Hz, 1H), 7.54 (d, J=8.0 Hz, 1H), 7.32 (t, J=7.7 Hz, 1H), 6.98 (t, J=7.5 Hz, 1H), 3.13 (d, J=5.2 Hz, 2H), 2.44 (t, J=7.4 Hz, 2H), 1.80-1.73 (m, 2H), 1.54-1.47 (m, 2H), 1.45 (s, 9H), 1.42-1.34 (m, 4H). Obtained a partially purified product that was used in the next step.

### Tert-butyl (7-((3-bromophenyl) amino)-7-oxoheptyl)carbamate (40)

White solid, 420 mg (52.6%) mp. 90-95° C. ¹H NMR (600 MHz, CDCl₃) δ 7.83 (s, 1H), 7.47 (d, J=7.0 Hz, 1H), 7.23 (d, J=7.8 Hz, 1H), 7.17 (t, J=8.0 Hz, 1H), 3.13 (d, J=5.5 Hz, 2H), 2.35 (t, J=7.3 Hz, 2H), 1.77-1.70 (m, 2H), 1.53-1.47 (m, 2H), 1.46 (s, 9H), 1.42-1.33 (m, 4H). ¹³C NMR (151 MHz, CDCl₃): δ 171.9, 156.3, 139.6, 130.2, 126.9, 122.7, 122.5, 118.2, 79.3, 40.2, 37.2, 30, 28.5, 28.3, 26, 25.4.

### Tert-butyl (7-((6-chlorobenzo[d]thiazol-2-yl)amino)-7-oxoheptyl)carbamate (41)

¹H NMR (400 MHz, CDCl₃) δ 7.80 (s, 1H), 7.65 (d, J=8.6 Hz, 1H), 7.40 (d, J=8.6 Hz, 1H), 3.13 (q, J=5.3 Hz, 2H), 2.52 (t, J=7.3 Hz, 2H), 1.83-1.73 (m, 2H), 1.55-1.49 (m, 2H), 1.45 (s, 9H), 1.43-1.33 (m, 4H). Obtained a partially purified product that was used in the next step.

### Tert-butyl (7-((6-fluorobenzo[d]thiazol-2-yl) amino)-7-oxoheptyl)carbamate (42)

White solid, 400 mg (50.6%) mp. 150-155° C. ¹H NMR (400 MHz, CDCl₃) δ 7.69 (dd, J=8.8, 4.7 Hz, 1H), 7.52 (dd, J=8.1, 2.5 Hz, 1H), 7.17 (td, J=8.9, 2.6 Hz, 1H), 3.12 (q, J=6.3 Hz, 2H), 2.49 (t, J=7.3 Hz, 2H), 1.83-1.70 (m, 2H), 1.53-1.47 (m, 2H), 1.45 (s, 9H), 1.41-1.30 (m, 4H). ¹³C NMR (151 MHz, CDCl₃) δ 171.8, 160.4, 158.8, 156, 144.2, 133 (d, J=10.5 Hz), 121.3 (d, J=8.6 Hz), 114.7 (d, J=24.6 Hz), 108 (d, J=26.6 Hz), 79.2, 40.4, 36.3, 29.9, 28.6, 28.5, 26.3, 24.8.

### Tert-butyl (7-((6-bromobenzo[d]thiazol-2-yl) amino)-7-oxoheptyl)carbamate (43)

Yellow solid, 160 mg (17.%) mp. 180-185° C. ¹H NMR (600 MHz, DMSO) δ 8.25 (s, 1H), 7.67 (d, J=8.5 Hz, 1H), 7.57 (d, J=8.2 Hz, 1H), 6.79 (s, 1H), 2.89 (d, J=5.8 Hz, 2H), 2.48 (t, J=7.1 Hz, 2H), 1.64-1.57 (m, 2H), 1.37 (s, 9H), 1.32-1.20 (m, 4H). ¹³C NMR (151 MHz, DMSO) δ 173, 159.2, 156, 148.2, 134.1, 129.6, 124.7, 122.5, 115.8, 77.8, 35.5, 29.8, 28.7, 26.5, 24.9.

### Tert-butyl (7-((5-bromobenzo[d]oxazol-2-yl) amino)-7-oxoheptyl)carbamate (44)

Brown solid, 437 mg, (49.6%) mp. 140-145° C. ¹H NMR (400 MHz, CDCl₃) δ 7.73 (s, 1H), 7.39-7.32 (m, 2H), 3.13 (q, J=6.3 Hz, 2H), 2.75-2.61 (m, 2H), 1.83-1.72 (m, 2H), 1.55-1.47 (m, 2H), 1.45 (s, 9H), 1.41-1.25 (m, 4H). ¹³C NMR (151 MHz, CDCl₃) δ 171.3, 156.4, 156.1, 147.2, 126.7, 121.4, 117.5, 111.5, 79.2, 40.4, 36.7, 29.9, 28.6, 28.5, 26.3, 24.9, 24.6.

### Tert-butyl (7-(benzo[d]oxazol-2-ylamino)-7-oxoheptyl) carbamate (45)

¹H NMR (400 MHz, CDCl₃) δ 7.60 (d, J=6.8 Hz, 1H), 7.47 (d, J=8.2 Hz, 1H), 7.32 (t, J=7.0 Hz, 1H), 7.23 (t, J=9.5 Hz, 1H), 3.14 (q, J=6.6 Hz, 2H), 2.78-2.63 (m, 2H), 1.84-1.73 (m, 2H), 1.57-1.48 (m, 2H), 1.46 (s, 9H), 1.43-1.30 (m, 4H). Obtained a partially purified product that was used in the next step.

### Tert-butyl(7-((6-methoxybenzo[d]thiazol-2-yl) amino)-7-oxoheptyl)carbamate (46)

White solid, 446 mg (54. %) mp. 105-110° C. ¹H NMR (400 MHz, CDCl₃) δ 7.60 (d, J=8.9 Hz, 1H), 7.30 (d, J=2.4 Hz, 1H), 7.03 (dd, J=8.9, 2.5 Hz, 1H), 3.86 (s, 3H), 3.05 (q, J=5.7 Hz, 2H), 1.71-1.61 (m, 2H), 1.52-1.46 (m, 2H), 1.43 (s, 9H), 1.32-1.17 (m, 4H). ¹³C NMR (151 MHz, CDCl₃): δ 171.6, 157.3, 156.9, 156, 142, 133.2, 121, 115.3, 104.4, 79.1, 55.9, 40.4, 36.3, 29.9, 28.7, 28.5, 26.4, 24.9.

### Tert-butyl(7-((5-methoxybenzo[d]thiazol-2-yl) amino)-7-oxoheptyl)carbamate (47)

White solid, 571 mg (70%) mp. 85-90° C. ¹H NMR (400 MHz, CDCl₃): δ 7.69 (d, J=8.6 Hz, 2H), 7.24 (s, 1H), 6.97 (d, J=8.7 Hz, 2H), 3.86 (s, 3H), 3.06 (d, J=6.4 Hz, 2H), 2.46 (t, J=6.5 Hz, 2H), 1.75-1.63 (m, 2H), 1.54-1.47 (m, 2H), 1.44 (s, 9H), 1.35-1.19 (m, 4H). ¹³C NMR (151 MHz, CDCl₃): δ 171.8, 160.7, 159.1, 156, 149.1, 123.8, 122, 113.1, 104.4, 79.1, 55.7, 40.4, 36.3, 29.9, 28.8, 28.5, 26.4, 24.9.

### Tert-butyl(7-((5-(4-methoxyphenyl) thiazol-2-yl)amino)-7-oxoheptyl)carbamate (48)

¹H NMR (400 MHz, CDCl₃) δ 7.73 (d, J=8.9 Hz, 2H), 7.00 (s, 1H), 6.95 (d, J=8.8 Hz, 2H), 3.85 (s, 3H), 3.18-3.06 (m, 2H), 2.33 (t, J=7.5 Hz, 2H), 1.79-1.60 (m, 2H), 1.45 (s, 9H), 1.34-1.21 (m, 4H). Obtained a partially purified product that was used in the next step.

### Tert-butyl(7-((5-(4-bromophenyl) thiazol-2-yl) amino)-7-oxoheptyl)carbamate (49)

White solid, 374 mg (38.7%) mp. 140-145° C. ¹H NMR (600 MHz, CDCl₃) δ 7.64 (d, J=7.5 Hz, 2H), 7.51 (d, J=7.5 Hz, 2H), 7.12 (s, 1H), 3.14-3.04 (m, 2H), 2.23-2.12 (m, 2H), 1.70-1.48 (m, 2H), 1.39 (s, 9H), 1.35-1.03 (m, 4H). ¹³C NMR (151 MHz, CDCl₃): δ 171.3, 159, 156.1, 148.3, 133.2, 132, 127.7, 122, 108.3, 79.2, 40.4, 35.9, 29.9, 28.6, 28.5, 26.3, 24.8.

### Tert-butyl(7-oxo-7-((5-(4-(((2,2,2-trichloroethoxy)carbonyl)oxy)phenyl)thiazol-2-yl)amino)heptyl)carbamate (50)

White solid, 520 mg (44%) mp. 140-145° C. ¹H NMR (600 MHz, CDCl₃) δ 7.88 (d, J=8.8 Hz, 2H), 7.30 (d, J=8.7 Hz, 2H), 7.16 (s, 1H), 4.91 (s, 2H), 3.13 (d, J=5.2 Hz, 2H), 2.37 (t, J=7.2 Hz, 2H), 1.79-1.63 (m, 2H), 1.47 (s, 9H), 1.38-1.23 (m, 4H). ¹³C NMR (151 MHz, CDCl₃): δ 170.9, 158.3, 152.5, 150.6, 148.3, 132.6, 127.3, 121.2, 108.1, 94.1, 79.2, 40.3, 36, 29.9, 28.6, 28.5, 26.3, 24.8.

### Synthesis of 51

A mixture of tert-butyl(7-oxo-7-((5-(4-(((2,2,2-trichloroethoxy)carbonyl) oxy)phenyl) thiazol-2-yl)amino) heptyl)carbamate (500 mg, 0.84 mmol) was dissolved in 19 ml of methanol, followed by addition of 1 ml 2M NaOH (aq.) (2 mmol, 2.38 equiv) and the reaction was continued until completion, monitored by TLC. The solvent was removed under reduced pressure and the water layer was first cooled using an ice bath, followed by slow addition of conc. HCl until a white solid was obtained. The solid was filtered and dried under vacuum to yield 7-((5-(4-hydroxyphenyl) thiazol-2-yl) amino)-7-oxoheptan-1-aminium chloride as a crude product that was used in the next reaction without further purification. ¹H NMR (600 MHz, CD₃OD): δ 7.70 (d, J=8.6 Hz, 2H), 6.86 (d, J=8.6 Hz, 2H), 2.95 (t, J=7.1 Hz, 2H), 2.61 (t, J=7.3 Hz, 2H), 1.84-1.75 (m, 2H), 1.73-1.68 (m, 2H), 1.51-1.45 (m, 4H).

### Synthesis of 52

A mixture of tert-butyl(7-oxo-7-((5-(4-(((2,2,2-trichloroethoxy)carbonyl) oxy)phenyl) thiazol-2-yl) amino) heptyl) carbamate (440 mg, 0.742 mmol) and sodium hydroxide (31 mg, 0.78 mmol, 1.05 equiv, 0.02 M) was stirred in methanol (39 ml) at room temperature for 3 hours, after which the solvent was removed under reduced pressure, water was added to the reaction mixture (5 mL), and the reaction mixture was washed with ethyl acetate (5 mL). The aqueous layer was slowly acidified and extracted with ethyl acetate (5 mL) three times. The organic extract was dried (Na₂SO₄) concentrated and purified using flash chromatography (ethyl acetate/hexanes 30-70%) to give tert-butyl (7-((5-(4-hydroxyphenyl) thiazol-2-yl) amino)-7-oxoheptyl) carbamate (183 mg, 0.44 mmol) as a partially purified product that was used in the next step. ¹H NMR (600 MHz, CD₃OD) δ 7.71 (d, J=8.7 Hz, 2H), 7.20 (s, 1H), 6.84 (d, J=8.7 Hz, 2H), 3.05 (t, J=7.0 Hz, 2H), 2.54 (t, J=7.5 Hz, 2H), 1.79-1.71 (m, 2H), 1.54-1.48 (m, 2H), 1.45 (s, 9H), 1.44-1.35 (m, 4H).

### Synthesis of TFPAs

### General Procedure for Preparation of Linker Variants of TFPAs

To a 10 mL round bottom flask was added N,N-dimethyl formamide (1 mL), trifluoro pyruvic acid (160 mg, 1 mmol, 1 equiv), HATU (380 mg, 1 mmol, eq,), HOBt (135 mg, 1 mmol, 1 equiv), NMM (330 μl, 3 mmol, 3 equiv), followed by 4a-4d (1 mmol, 1 equiv) and molecular beads. The mixture was stirred for four hours, ethyl acetate was added to the mixture (5 mL), and the mixture was washed several times with ice water (3 mL). The organic layer was dried (Na₂SO₄) and the mixture was concentrated in vacuo, and purified by flash chromatography (MeOH/DCM 1.5-2%) to yield the respective TFPAs.

### N-phenyl-5-(3,3,3-trifluoro-2,2-dihydroxypropanamidopentanamide (10a)

White solid, 64 mg (19.1%) mp. 108-113° C. ¹H NMR (600 MHz, CD₃OD): δ 7.56 (d, J=7.6 Hz, 2H), 7.31 (t, J=7.5 Hz, 2H), 7.10 (t, J=7.4 Hz, 1H), 3.34-3.32 (m, 2H), 2.42 (t, J=7.4 Hz, 2H), 1.79-1.71 (m, 2H), 1.68-1.57 (m, 2H). ¹³C NMR (151 MHz, CD₃OD): δ 172.9, 165.3, 138.5, 128.4, 123.7, 121.8 (q, J=287.3 Hz), 119.80, 94.3 (q, J=32 Hz), 38.8, 35.9, 28.4, 22.6. HRMS-ESI M/z: [M+H]⁺ calcd. 335.1218 for C₁₄H₁₇F₃N₂O₄. found 335.1218, 99% purity.

### N-phenyl-6-(3,3,3-trifluoro-2,2-dihydroxypropanamido)hexanamide (10b)

White solid, 41 mg (11.8%) mp. 103-108° C. ¹H NMR (400 MHz, CD₃OD) δ 7.53 (d, J=7.6 Hz, 2H), 7.28 (t, J=8.0 Hz, 2H), 7.07 (t, J=7.4 Hz, 1H), 3.26 (q, J=7.0 Hz, 2H), 2.36 (t, J=7.4 Hz, 2H), 1.85-1.64 (m, 2H), 1.64-1.52 (m, 2H), 1.52-1.33 (m, 2H). ¹³C NMR (151 MHz, CD₃OD): δ 173.1, 165.2, 138.5, 128.4, 123.7, 121.8 (q, J=287.2 Hz), 119.8, 94.3, (q, J=32 Hz), 39, 36.4, 28.6, 26.1, 25.1. HRMS-ESI M/z: [M+H]⁺ calcd. 349.1375 for C₁₅H₁₉F₃N₂O₄. found 349.136, 99% purity.

### N-phenyl-7-(3,3,3-trifluoro-2,2-dihydroxypropanamido)heptanamide (10c)

White solid, 72 mg (26.4%) mp. 123-128° C. ¹H NMR (600 MHz, CD₃OD) δ 7.55 (d, J=7.5 Hz, 2H), 7.31 (t, J=8.4 Hz, 2H), 7.09 (t, J=7.4 Hz, 1H), 3.28 (q, J=7.0 Hz, 2H), 2.38 (t, J=7.5 Hz, 2H), 1.77-1.68 (m, 2H), 1.64-1.53 (m, 2H), 1.47-1.32 (m, 4H). ¹³C NMR (151 MHz, CD₃OD): δ 172.2, 165.2, 138.5, 128.4, 123.7, 121.8, (q, J=287.3 Hz), 119.9, 94.3 (q, J=32 Hz), 39.1, 36.5, 28.7, 28.5, 26.2, 25.42. HRMS-ESI M/z: [M+H]⁺ calcd. 363.1531 for C₁₆H₂₁F₃N₂O₄. found 363.1531, 96% purity.

### N-phenyl-8-(3,3,3-trifluoro-2,2-dihydroxypropanamido)octanamide (10d)

White solid, 139 mg (37%) mp. 103-108° C. ¹H NMR (600 MHz, CD₃OD) δ 7.56 (d, J=7.7 Hz, 2H), 7.31 (t, J=8.0 Hz, 2H), 7.09 (t, J=7.4 Hz, 1H), 3.27 (q, J=6.1 Hz, 2H), 2.38 (t, J=7.5 Hz, 2H), 1.74-1.67 (m, 2H), 1.59-1.52 (m, 2H), 1.44-1.33 (m, 6H). ¹³C NMR (151 MHz, CD₃OD): δ 173.3, 165.1, 138.5, 128.4, 123.7, 121.82 (q, J=287.2 Hz), 119.9, 94.4 (q, J=32 Hz), 39.2, 36.5, 28.8, 28.77, 28.6, 26.3, 25.4. HRMS-ESI M/z: [M+H]⁺ calcd. 377.1688 for C₁₇H₂₃F₃N₂O₄. found 377.1688, 99% purity.

### General procedure for preparation of SAR variants of TFPAs (53-85)

To a 10 mL round bottom flask was added the respective boc-arene (0.4 mmol), followed by 20% trifluoroacetic acid in dichloromethane (1 mL: 4 mL), and the mixture was stirred at room temperature for four hours. The solvent was removed under reduced pressure, and the mixture was dried under vacuum, followed by addition of N,N-dimethyl formamide (1 mL), trifluoropyruvic acid (64 mg, 0.4 mmol, 1 equiv), HATU (152 mg, 0.4 mmol, eq,), HOBt (54 mg, 0.4 mmol, 1 equiv), NMM (132 μl, 1.2 mmol, 3 equiv). The mixture was stirred for four hours, ethyl acetate was added to the mixture (5 mL), and the mixture was washed several times with ice water (3 mL). The organic layer was dried (Na₂SO₄), the solvent was removed under reduced pressure, and the mixture was dissolved in silica and purified by flash chromatography (MeOH/DCM 1.5-2%) to yield the respective TFPAs (4-42%, 2 steps).

### N-phenyl-4-(3,3,3-trifluoro-2,2-dihydroxypropanamido)butanamide (14)

White solid, 64 mg, (50%) mp. 105-110° C. ¹HNMR (400 MHz, CD₃OD): δδ 7.56 (dd, J=8.5, 0.9 Hz, 2H), 7.31 (t, J=8.0 Hz, 2H), 7.09 (t, J=7.4 Hz, 1H), 3.33 (p, J=3.2 Hz, 2H), 2.42 (t, J=7.5 Hz, 2H), 1.93 (p, J=7.2 Hz, 2H). ¹³C NMR (151 MHz, CD₃OD): δ 172.3, 165.5, 138.4, 128.4, 123.8, 121.8 (q, J=287.4 Hz), 119.9, 94.3 (q, J=32 Hz), 38.8, 33.7, 24.9. HRMS-ESI M/z: [M+H]⁺ calcd. 321.1062 for C₁₃H₁₅F₃N₂O₄. found 321.1069, 99% purity.

### N-(4-acetylphenyl)-7-(3,3,3-trifluoro-2,2-dihydroxypropanamido)heptanamide (53)

White solid, 26 mg, (16.19%) mp. 133-138° C. ¹H NMR (400 MHz, CD₃OD) δ 7.95 (d, J=8.8 Hz, 2H), 7.71 (d, J=8.8 Hz, 2H), 3.26 (t, J=6.7 Hz, 2H), 2.56 (s, 3H), 2.40 (t, J=7.5 Hz, 2H), 1.75-1.65 (m, 2H), 1.60-1.50 (m, 2H), 1.45-1.32 (m, 4H). ¹³C NMR (151 MHz, CD₃OD): δ 173.5, 165.2, 143.4, 132.2, 129.3, 121.8 (q, J=287.4 Hz), 118.7, 94.3 (q, J=32 Hz), 39.1, 36.6, 28.7, 28.5, 26.2, 25.2, 25.1. HRMS-ESI M/z: [M+H]⁺ calcd. 405.1637 for C₁₈H₂₃F₃N₂O₅. found 405.1637, 98.9% purity.

### N-(4-(pentafluoro-λ⁶-sulfaneyl)phenyl)-7-(3,3,3-trifluoro-2,2dihydroxypropanamido)heptanamide (54)

White solid, 52 mg (26.6%) mp. 105-110° C. ¹H NMR (600 MHz, CD₃OD) δ 7.76 (s, 4H), 3.28 (td, J=7.0, 2.5 Hz, 2H), 2.42 (t, J=7.5 Hz, 2H), 1.77-1.67 (m, 2H), 1.62-1.54 (m, 2H), 1.46-1.35 (m, 4H). ¹³C NMR (151 MHz, CD₃OD): δ 173.5, 165.2 (d, J=4 Hz), 148.4 (m, J=17 Hz), 141.9, 126.5 (t, J=4 Hz), 121.8 (q, J=287.3 Hz), 118.7, 94.4 (q, J=32 Hz), 39.1, 36.5, 28.7, 28.5, 26.1, 25.1. HRMS-ESI M/z: [M+H]⁺ calcd. 489.1094 for C₁₆H₂₀F₈N₂O₄S. found 489.1093, 99% purity.

### N-(4-methoxyphenyl)-7-(3,3,3-trifluoro-2,2-dihydroxypropanamido)heptanamide (55)

White solid, 49 mg (31%) mp. 105-110° C. ¹H NMR (400 MHz, CD₃OD) δ 7.42 (d, J=9.0 Hz, 2H), 6.86 (d, J=9.0 Hz, 2H), 3.76 (s, 3H), 3.25 (t, J=6.9 Hz, 2H), 2.33 (t, J=7.5 Hz, 2H), 1.77-1.62 (m, 2H), 1.58-1.48 (m, 2H), 1.46-1.28 (m, 4H). ¹³C NMR (151 MHz, CD₃OD): δ 173, 165.2, 156.5, 132.4, 121.8 (q, J=287.2 Hz), 121.7, 113.5, 94.3 (q, J=32 Hz), 54.4, 39.1, 36.3, 28.7, 28.5, 26.2, 25.5. HRMS-ESI M/z: [M+H]⁺ calcd. 393.1637 for C₁₇H₂₃F₃N₂O₅. found 393.1634, 99% purity.

### N-(4-fluorophenyl)-7-(3,3,3-trifluoro-2,2-dihydroxypropanamido)heptanamide (56)

White solid, 64 mg (42%) mp. 97-103° C. ¹H NMR (400 MHz, CD₃OD): δ 7.56 (d, J=4.9 Hz, 2H), 7.05 (d, J=8.3 Hz, 2H), 3.31-3.24 (m, 2H), 2.37 (t, J=6.2 Hz, 2H), 1.75-1.67 (m, 2H), 1.61-1.53 (m, 2H), 1.47-1.35 (m, 4H). ¹³C NMR (151 MHz, CD₃OD): δ 173.1, 165.2 (d, J=4 Hz), 160, 158.4, 134.7 (d, J=2 Hz), 121.8 (q, J=287.2 Hz), 121.6, 114.9, 114.7, 94.4 (q, J=32 Hz), 54.4, 39.1, 36.3, 28.7, 28.5, 26.2, 25.4. HRMS-ESI M/z: [M+H]⁺ calcd. 381.1437 for C₁₆H₂OF₄N₂O₄. found 381.1432, 96% purity.

### N-(pyridin-4-yl)-7-(3,3,3-trifluoro-2,2-dihydroxypropanamido)heptanamide (57)

White solid, 19 mg (13.2%) mp. 150-155° C. ¹H NMR (400 MHz, CD₃OD) δ 8.37 (d, J=5.5 Hz, 2H), 7.64 (d, J=6.3 Hz, 2H), 3.25 (t, J=7.0 Hz, 2H), 2.41 (t, J=7.5 Hz, 2H), 1.76-1.63 (m, 2H), 1.63-1.48 (m, 2H), 1.45-1.31 (m, 4H). ¹³C NMR (151 MHz, CD₃OD): δ 174, 165.2, 149.3, 146.8, 121.8 (q, J=287.3 Hz), 113.6, 94.3 (q, J=32 Hz), 39.1, 36.6, 28.7, 28.4, 26.1, 25. HRMS-ESI M/z: [M+H]⁺ calcd. 364.1844 for C₁₅H₂₀F₃N₃O₄. found 364.1484, 99% purity.

### N—(S-phenylthiazol-2-yl)-7-(3,3,3-trifluoro-2,2-dihydroxypropanamido)heptanamide (58)

White solid, 18 mg (9.9%) mp. 143-148° C. ¹H NMR (400 MHz, CD₃OD): δ 7.91 (d, J=8.2 Hz, 2H), 7.40 (t, J=7.7 Hz, 2H), 7.37 (s, 1H), 7.31 (t, J=7.4 Hz, 1H), 3.28 (q, J=6.4 Hz, 2H), 2.51 (t, 2H), 1.77-1.72 (m, 2H), 1.62-1.55 (m, 2H), 1.47-1.38 (m, 4H). ¹³C NMR (151 MHz, CD₃OD): δ 172.4, 165.2, 158, 149.8, 134.6, 128.2, 128.1, 127.4, 127.2, 125.6, 125,56, 121.8 (q, J=287.4 Hz), 107.1, 101.4, 94.3 (q, J=32 Hz), 39.1, 35.1, 28.7, 28.4, 26.1, 24.9. HRMS-ESI M/z: [M+H]⁺ calcd. 446.1361 for C₁₉H₂₂F₃N₃O₄S. found 446.1376, 95% purity.

### N-(3-methoxyphenyl)-7-(3,3,3-trifluoro-2,2-dihydroxypropanamido)heptanamide (59)

White solid, 40 mg (26%) mp. 120-125° C. ¹H NMR (400 MHz, CD₃OD): δ 7.91 (s, 1H), 7.47 (d, J=7.1 Hz, 1H), 7.25-7.19 (m, 2H), 3.30-3.24 (m, 2H), 2.40-2.35 (m, 2H), 1.73-1.67 (m, 2H), 1.60-1.53 (m, 2H), 1.44-1.35 (m, 4H). ¹³C NMR (151 MHz, CD₃OD): δ 173.2, 165.2, 160.1, 139.7, 129.1, 121.8 (q, J=286.9 Hz), 111.9, 109.2, 105.6, 94.3 (q, J=32 Hz), 54.2, 39.1, 36.5, 28.7, 28.5, 26.2, 25.4. HRMS-ESI M/z: [M+H]⁺ calcd. 393.1637 for C₁₇H₂₃F₃N₂O₅. found 393.1644, 98% purity.

### N-(thiazol-2-yl)-7-(3,3,3-trifluoro-2,2-dihydroxypropanamido)heptanamide (60)

White solid, 55 mg (37%) mp. 137-142° C. ¹H NMR (600 MHz, CD₃OD): δ 7.43 (d, J=3.6 Hz, 2H), 7.11 (d, J=3.6 Hz, 2H), 3.31-3.24 (m, 2H), 2.50 (t, J=7.5 Hz, 2H), 1.76-1.69 (m, 2H), 1.61-1.54 (m, 2H), 1.45-1.36 (m, 4H). ¹³C NMR (151 MHz, CD₃OD): δ 172.2, 165.2, 158.7, 136.9, 121.8 (q, J=287.2 Hz), 113, 94.3 (q, J=32 Hz), 39.1, 36.5, 35, 28.7, 28.4, 26.1, 24.9. HRMS-ESI M/z: [M+H]⁺ calcd. 370.1048 for C₁₃H₁₈F₃N₃O₄S. found 370.1057, 99% purity.

### N-(benzo[d]thiazol-2-yl)-7-(3,3,3-trifluoro-2,2-dihydroxypropanamido)heptanamide (61)

White solid, 20 mg (12%) mp. 150-155° C. ¹H NMR (600 MHz, acetone-d&) δ 7.94 (d, J=7.9 Hz, 1H), 7.71 (d, J=8.1 Hz, 1H), 7.43 (t, J=8.3 Hz, 1H), 7.31 (t, J=8.1 Hz, 1H), 3.33 (q, J=6.9 Hz, 2H), 2.63 (t, J=7.5 Hz, 2H), 1.80-1.73 (m, 2H), 1.64-1.57 (m, 2H), 1.48-1.38 (m, 4H). ¹³C NMR (151 MHz, Acetone-d₆) δ 171.9, 166.5, 157.9, 149, 132.1, 125.9, 123.5, 122.58 (q, J=287.7 Hz), 121.3, 120.70, 90.8 (q, J=32.4 Hz), 39.6, 35.5, 26.1, 24.8. HRMS-ESI M/z: [M+H]⁺ calcd. 420.1204 for C₁₇H₂₀F₃N₃O₄S. found 420.1208, 98% purity.

### N-(2,4-difluorophenyl)-7-(3,3,3-trifluoro-2,2-dihydroxypropanamido)heptanamide (62)

White solid, 51 mg (32%) mp. 100-105° C. ¹H NMR (600 MHz, CD₃OD) δ 7.77 (td, J=8.9, 6.1 Hz, 1H), 7.07-7.00 (m, 1H), 6.99-6.93 (m, 1H), 3.29 (q, J=6.9 Hz, 2H), 2.43 (t, J=7.5 Hz, 2H), 1.75-1.68 (m, 2H), 1.62-1.55 (m, 2H), 1.47-1.36 (m, 4H). ¹³C NMR (151 MHz, CD₃OD): δ 173.7, 165.2, 160.6 (d, J=11.4 Hz), 159.01 (d, J=11.4 Hz), 155.88 (d, J=12.2 Hz), 154.23 (d, J=12.2 Hz), 126.21 (dd, J=9.5, 2.5 Hz), 121.98 (dd, J=12.2, 3.8 Hz), 121.80 (q, J=287.3 Hz), 110.50 (dd, J=22.2, 3.7 Hz), 103.44 (dd, J=26.7, 24.3 Hz), 94.34 (d, J=32.1 Hz), 39.1, 35.7, 28.69, 28.4, 26.1, 25.3. HRMS-ESI M/z: [M+H]⁺ calcd. 399.1343 for C₁₆H₁₉F₅N₂O₄. found 399.1327, 98% purity.

### 7-(3,3,3-trifluoro-2,2-dihydroxypropanamido)-N-(2,4,6-trifluorophenyl)heptanamide (63)

White solid, 23 mg (13.8%) mp. 95-100° C. ¹H NMR (600 MHz, CD₃OD) δ 6.96 (t, J=8.3 Hz, 1H), 3.32-3.25 (m, 2H), 2.44 (t, J=7.4 Hz, 2H), 1.76-1.69 (m, 2H), 1.61-1.55 (m, 2H), 1.49-1.37 (m, 4H). ¹³C NMR (151 MHz, CD₃OD): δ 25.34, 26.13, 28.31, 28.72, 35.13, 39.28, 174.1, 165.3 (d, J=4.2 Hz), 161.88 (t, J=15 Hz), 160.23 (t, J=15 Hz), 159.5 (dd, J=15.4, 7.4 Hz), 157.8 (dd, J=15.4, 7.4 Hz), 121.8 (q, J=287.2 Hz), 110.7 (td, J=17.3. 5.1 Hz), 101.1-99.2 (m), 94.4 (m, J=32 Hz), 39.3, 35.1, 28.7, 28.3, 26.1, 25.3. HRMS-ESI M/z: [M+H]⁺ calcd. 417.1249 for C₁₆H₁₈F₆N₂O₄. found 417.1256, 95% purity.

### N-(4-chlorophenyl)-7-(3,3,3-trifluoro-2,2-dihydroxypropanamido)heptanamide (64)

White solid, 27 mg (17%) mp. 103-108° C. ¹H NMR (400 MHz, CD₃OD) δ 7.55 (d, J=8.8 Hz, 2H), 7.28 (d, J=8.8 Hz, 2H), 3.26 (t, J=7.2 Hz, 2H), 2.35 (t, J=7.5 Hz, 2H), 1.74-1.63 (m, 2H), 1.61-1.50 (m, 2H), 1.44-1.30 (m, 4H). ¹³C NMR (151 MHz, CD₃OD): δ 173.2, 165.2, 137.4, 128.5, 128.3, 121.8 (q, J=287.2 Hz), 121.1, 94.3 (q, J=32 Hz), 39.1, 36.4, 28.7, 28.5, 26.1, 25.3. HRMS-ESI M/z: [M+H]⁺ calcd. 397.1141 for C₁₆H₂₀ClF₃N₂O₄. found 397.1169, 95% purity.

### N-(4-bromophenyl)-7-(3,3,3-trifluoro-2,2-dihydroxypropanamido)heptanamide (65)

White solid, 19 mg (10.8%) mp. 105-110° C. ¹H NMR (400 MHz, CD₃OD) δ 7.50 (d, J=8.8 Hz, 2H), 7.42 (d, J=8.9 Hz, 2H), 3.25 (t, J=7.0 Hz, 2H), 2.35 (t, J=7.5 Hz, 2H), 1.73-1.63 (m, 2H), 1.60-1.51 (m, 2H), 1.44-1.32 (m, 4H). ¹³C NMR (151 MHz, CD₃OD) δ 173.2, 165.2, 137.9, 131.3, 121.8 (q, J=287.2 Hz), 121.37, 115.9, 94.3 (q, J=32 Hz), 39.1, 36.4, 28.7, 28.5, 26.1, 25.3. HRMS-ESI M/z: [M+H]⁺ calcd. 441.0636 for C₁₆H₂₀BrF₃N₂O₄. found 441.0641, 99% purity.

### N-(2-methoxyphenyl)-7-(3,3,3-trifluoro-2,2-dihydroxypropanamido)heptanamide (66)

White solid, 18 mg (11.7%) mp. 105-110° C. ¹H NMR (400 MHz, CD₃OD) δ 7.90 (d, J=7.9 Hz, 1H), 7.08 (t, J=8.6 Hz, 1H), 6.98 (d, J=7.4 Hz, 1H), 6.89 (t, J=8.2 Hz, 1H), 3.24 (q, J=7.9 Hz, 2H), 2.41 (t, J=7.5 Hz, 2H), 1.74-1.63 (m, 2H), 1.59-1.50 (m, 2H), 1.44-1.32 (m, 4H). ¹³C NMR (151 MHz, CD₃OD): δ 173.3, 165.2, 150.3, 126.7, 124.8, 122.3, 121.8 (q, J=287.3), 120, 110.4, 94.3 (q, J=32 Hz), 54.81, 54.80, 39.2, 36.3, 28.7, 28.4, 26.2, 25.5. HRMS-ESI M/z: [M+H]⁺ calcd. 393.1637 for C₁₇H₂₃F₃N₂O₅. found 393.1636, 99% purity.

### N-(4-methyl-S-phenylthiazol-2-yl)-7-(3,3,3-trifluoro-2,2-dihydroxypropanamido)heptanamide (67)

White solid, 36 g (20%) mp. 143-148° C. ¹H NMR (400 MHz, acetone-d₆) δ 7.66 (d, J=8.4 Hz, 2H), 7.42 (t, J=7.7 Hz, 2H), 7.31 (t, J=7.4 Hz, 1H), 3.31 (q, J=6.5 Hz, 2H), 2.55 (t, J=7.4 Hz, 2H), 2.51 (s, 3H), 1.77-1.68 (m, 3H), 1.63-1.54 (m, 2H), 1.46-1.35 (m, 4H). ¹³C NMR (151 MHz, Acetone-d₆): δ170.9, 166.4, 154, 135.6, 128.2, 128.1, 127, 122.6 (q, J=287.7 Hz), 121.1, 90.7 (q, J=32.5 Hz), 39.6, 35.3, 26.2, 24.9, 11.3. HRMS-ESI M/z: [M+H]⁺ calcd. 460.1517 for C₂₀H₂₄F₃N₃O₄S. found 460.1506, 99% purity.

### N-(4,5-diphenylthiazol-2-yl)-7-(3,3,3-trifluoro-2,2-dihydroxypropanamido)heptanamide (68)

White solid, 22 mg (10.6%) mp. 143-148° C. ¹H NMR (400 MHz, CD₃OD): δ ¹H NMR (600 MHz, CD₃OD) δ 7.49-7.45 (m, 1H), 7.36-7.31 (m, 3H), 7.27 (dd, J=3.7, 2.5 Hz, 1H), 3.28 (dt, J=12.0, 6.9 Hz, 2H), 2.52 (t, J=7.4 Hz, 2H), 1.79-1.71 (m, 2H), 1.62-1.56 (m, 2H), 1.48-1.38 (m, 4H). ¹³C NMR (151 MHz, CD₃OD) δ 172.5, 165.2, 156.1, 144.3, 135, 132.29, 129.3, 128.6, 128.5, 127.8, 127.6, 127.4, 126.3, 121.8 (q, J=287.2 Hz), 94.3 (q, J=32 Hz), 39.1, 35.1, 28.7, 28.4, 26.1, 24.9. HRMS-ESI M/z: [M+H]⁺ calcd. 522.1674 for C₂₅H₂₆F₃N₃O₄S. found 522.1673, 99% purity.

### N-([1,1′-biphenyl]-4-yl)-7-(3,3,3-trifluoro-2,2-dihydroxypropanamido)heptanamide (69)

White solid, 32 mg (18.2%) mp. 123-12 8° C. ¹H NMR (600 MHz, acetone-d₆): δ 7.77 (d, J=8.5 Hz, 2H), 7.68-7.57 (m, 4H), 7.43 (t, J=7.7 Hz, 2H), 7.32 (t, J=7.4 Hz, 1H), 3.31 (q, J=6.7 Hz, 2H), 2.38 (t, J=7.4 Hz, 2H), 1.75-1.65 (m, 2H), 1.65-1.52 (m, 2H), 1.46-1.34 (m, 4H). ¹³C NMR (151 MHz, Acetone-d₆): δ 171.1, 166.4, 140.6, 139.1, 135.6, 128.8, 127, 126.9, 126.4, 122.6 (q, J=287.9 Hz), 119.4, 90.8 (q, J=32.5 Hz), 39.6, 36.8, 26.2, 25.3. HRMS-ESI M/z: [M+H]⁺ calcd. 439.1844 for C₂₂H₂₃F₃N₂O₄. found 439.1844, 99% purity.

### N-(2,5-dimethoxyphenyl)-7-(3,3,3-trifluoro-2,2-dihydroxypropanamido)heptanamide (70)

White solid, 17 mg (10%) mp. 108-113° C. ¹H NMR (400 MHz, acetone) δ 8.1 (d, J=2.5 Hz, 1H), 6.89 (d, J=8.9 Hz, 1H), 6.67 (s, 1H), 6.56 (dd, J=8.9, 3.0 Hz, 1H), 3.80 (s, 3H), 3.72 (s, 3H), 3.31 (q, J=6.8 Hz, 2H), 2.44 (t, J=7.4 Hz, 2H), 1.72-1.63 (m, 2H), 1.62-1.52 (m, 2H), 1.44-1.32 (m, 4H). ¹³C NMR (151 MHz, Acetone-d₆): δ 171.1, 166.4, 153.8, 142.6, 129.1, 122.6 (q, J=285.87 Hz), 111, 107, 106.8, 90.8 (q, J=32.5 Hz), 55.7, 55, 39.6, 36.9, 26.2, 25.3. HRMS-ESI M/z: [M+H]⁺ calcd. 423.1742 for C₁₈H₂₅F₃N₂O₆. found 423.1745, 99% purity.

### 7-(3,3,3-trifluoro-2,2-dihydroxypropanamido)-N-(3,4,5-trimethoxyphenyl)heptanamide (71)

White solid, 29 mg (15.9%) mp. 98-103° C. ¹H NMR (400 MHz, CD₃OD) δ 6.95 (s, 2H), 3.82 (s, 3H), 3.72 (s, 3H), 3.27 (t, J=7.2 Hz, 2H), 2.35 (t, J=7.5 Hz, 2H), 1.74-1.64 (m, 2H), 1.61-1.52 (m, 2H), 1.45-1.33 (m, 4H). ¹³C NMR (151 MHz, CD₃OD): ¹³C NMR (151 MHz, CD₃OD) δ 173.1, 165.2, 153, 135, 134, 121.8 (q, J=287.2 Hz), 94.3 (q, J=33 Hz), 97.4, 59.8, 55.1, 39.11, 36.5, 28.7, 28.5, 26.2, 25.4. HRMS-ESI M/z: [M+H]⁺ calcd. 453.1848 for C₁₉H₂₇F₃N₂O₇. found 453.1866, 99% purity.

### N-(naphthalen-1-yl)-7-(3,3,3-trifluoro-2,2-dihydroxypropanamido)heptanamide (72)

White solid, 48 mg (29%) mp. 123-128° C. ¹H NMR (400 MHz, CD₃OD) δ 7.97 (d, J=9.0 Hz, 1H), 7.90 (d, J=9.2 Hz, 1H), 7.78 (d, J=8.2 Hz, 1H), 7.59-7.45 (m, 4H), 3.29-3.22 (m, 2H), 2.55 (t, J=7.5 Hz, 2H), 1.86-1.75 (m, 2H), 1.65-1.56 (m, 2H), 1.55-1.39 (m, 4H). ¹³C NMR (151 MHz, CD₃OD): δ 174.4, 165.22, 165.19, 134.3, 132.8, 128.8, 128, 126.2, 125.9, 125.7, 125.1, 122.9, 122, 121.8 (q, J=287.4 Hz), 94.3 (q, J=32 Hz), 39.2, 35.6, 28.7, 28.6, 26.2, 25.6. HRMS-ESI M/z: [M+H]⁺ calcd. 413.163 for C₂₀H₂₃F₃N₂O₄. found 413.1428, 99% purity.

### N-(4-benzoylphenyl)-7-(3,3,3-trifluoro-2,2-dihydroxypropanamido)heptanamide (73)

Yellow solid, 24 mg (12.9%) mp. 113-118° C. ¹H NMR (600 MHz, CD₃OD): δ 7.80-7.73 (m, 6H), 7.64 (t, J=7.4 Hz, 1H), 7.54 (t, J=7.7 Hz, 2H), 3.30 (t, J=7.2 Hz, 2H), 2.44 (t, J=7.5 Hz, 2H), 1.77-1.70 (m, 2H), 1.64-1.57 (m, 2H), 1.48-1.37 (m, 4H). ¹³C NMR (151 MHz, CD₃OD): δ 196.2, 173.5, 157.5 (q, J=36.5 Hz), 143.1, 137.8, 132.2, 132.1, 131.1, 129.4, 128.1, 118.6, 116.9 (q, J=286.6 Hz), 39.3, 36.6, 28.5, 28.3, 26.1, 25.2. HRMS-ESI M/z: [M+Na]⁺ calcd. 489.1613 for C₂₃H₂₄F₃N₂NaO₅. found 489.1623, 96% purity.

### N-(4-((4-methylphenyl)sulfonamido)phenyl)-7-(3,3,3-trifluoro-2,2-dihydroxypropanamido) heptanamide (74)

White solid, 24 mg (11. %) mp. 115-120° C. ¹H NMR (400 MHz, CD₃OD) δ 7.58 (d, J=8.3 Hz, 2H), 7.38 (d, J=8.9 Hz, 2H), 7.26 (d, J=7.3 Hz, 2H), 6.98 (d, J=8.9 Hz, 2H), 3.24 (t, J=6.8 Hz, 2H), 2.36 (s, 3H), 2.31 (t, J=7.4 Hz, 2H), 1.72-1.60 (m, 2H), 1.59-1.48 (m, 2H), 1.43-1.26 (m, 4H). ¹³C NMR (151 MHz, CD₃OD): δ 173.1, 165.2, 143.6, 136.6, 135.6, 133.4, 129.1, 126.9, 122.1, 121.8 (q, J=287.2 Hz), 120.38, 94.4 (q, J=32.5 Hz), 39.1, 36.4, 28.7, 28.5, 26.1, 25.4, 20. HRMS-ESI M/z: [M+H]⁺ calcd. 532.167 for C₂₃H₂₈F₃N₃O₆S. found 532.1673, 98% purity.

### N-(2-bromophenyl)-7-(3,3,3-trifluoro-2,2-dihydroxypropanamido)heptanamide (75)

White solid, 8 mg (4.5%) mp. 108-113° C. ¹H NMR (400 MHz, CD₃OD) δ 7.62 (t, J=7.4 Hz, 2H), 7.35 (t, J=7.7 Hz, 1H), 7.12 (t, J=7.7 Hz, 1H), 3.27 (t, J=7.0 Hz, 2H), 2.44 (t, 2H), 1.79-1.67 (m, 2H), 1.62-1.51 (m, 2H), 1.50-1.33 (m, 4H). ¹³C NMR (151 MHz, CD₃OD): δ 173.6, 165.2, 135.8, 132.5, 127.6, 127, 126.9, 121.8 (q, J=287.2 Hz), 118.1, 94.3 (q, J=32.9 Hz), 39.2, 39.1, 35.9, 28.7, 28.5, 28.47, 26.2, 26.1, 25.4. HRMS-ESI M/z: [M+H]⁺ calcd. 441.0636 for C₁₆H₂₀BrF₃N₂O₄. found 441.0651, 99% purity.

### N-(3-bromophenyl)-7-(3,3,3-trifluoro-2,2-dihydroxypropanamido)heptanamide (76)

27 mg (15.3%). ¹H NMR (600 MHz, CD₃OD): δ 7.91 (s, 1H), 7.47 (d, J=7.1 Hz, 1H), 7.25-7.19 (m, 2H), 3.30-3.24 (m, 2H), 2.38 (t, J=7.5 Hz, 2H), 1.73-1.67 (m, 2H), 1.60-1.53 (m, J=9.1, 4.8 Hz, 2H), 1.44-1.35 (m, J=2.6 Hz, 5H). ¹³C NMR (151 MHz, CD₃OD): δ 173.4, 173.3, 165.2, 140.1, 129.9, 126.4, 122.4, 121.9, 121.8 (q, J=287 Hz), 118.1, 94.3 (q, J=32 Hz), 39.1, 36.4, 28.7, 28.5, 26.1, 25.3. HRMS-ESI M/z: [M+H]⁺ calcd. 441.0636 for C₁₆H₂₀BrF₃N₂O₄. found 441.063, 95% purity.

### N-(6-fluorobenzo[d]thiazol-2-yl)-7-(3,3,3-trifluoro-2,2-dihydroxypropanamido)heptanamide (77)

White solid, 20 mg (11.4%) mp. 146-151° C. ¹H NMR (600 MHz, CD₃OD): δ 1.42 (m, 4H, CH₂, J=5.64 Hz), 1.58 (m, 2H, CH₂, J=7.14 Hz), 1.75 (m, 2H, CH₂, J=7.5 Hz), 2.54 (t, 2H, CH₂, J=7.5 Hz), 3.29 (q, 2H, CH₂, J=4 Hz), 7.21 (td, 1H, CH, J=2.58, 9 Hz), 7.65 (dd, 1H, CH, J=2.58, 8.4 Hz), 7.72 (dd, 1H, CH, J=4.2, 4.68 Hz). ¹³C NMR (151 MHz, CD₃OD): δ 173, 165.2, 160.4, 158.8, 158.2, 145.2, 133.1 (d, J=10.8 Hz), 121.8 (q, J=287.2 Hz), 121.4 (d, J=9.1 Hz), 113.8 (d, J=24.8 Hz), 107.1 (d, J=27 Hz), 39.1, 35.2, 28.7, 28.4, 26.1, 24.7. HRMS-ESI M/z: [M+H]⁺ calcd. 438.1110 for C₁₇H₁₉F₄N₃O₄S. found 438.1111, 98% purity.

### N-(6-chlorobenzo[d]thiazol-2-yl)-7-(3,3,3-trifluoro-2,2-dihydroxypropanamido)heptanamide (78)

White solid, 13 mg (7.2%) mp. 165-170° C. ¹H NMR (400 MHz, CD₃OD) δ 7.89 (d, J=2.1 Hz, 1H), 7.68 (d, J=8.6 Hz, 1H), 7.40 (dd, J=8.7, 2.1 Hz, 1H), 3.26 (t, J=8.0 Hz, 2H), 2.52 (t, J=7.5 Hz, 2H), 1.77-1.67 (m, 2H), 1.61-1.50 (m, 2H), 1.47-1.34 (m, 4H). ¹³C NMR (151 MHz, CD₃OD): S 173.1, 165.2, 159, 147.4, 133.5, 128.7, 126.3, 121.8 (q, J=287.2 Hz), 121.4, 120.6, 39.1, 35.2, 28.7, 28.4, 26.1, 24.7. HRMS-ESI M/z: [M+H]⁺ calcd. 454.0815 for C₁₇H₁₉ClF₃N₃O₄S. found 454.0817, 98% purity.

### N-(6-bromobenzo[d]thiazol-2-yl)-7-(3,3,3-trifluoro-2,2-dihydroxypropanamido)heptanamide (79)

White solid, 11 mg (5.5%) mp. 163-168° C. ¹H NMR (600 MHz, acetone-d₆): δ 8.16 (d, J=2.0 Hz, 1H), 7.64 (d, J=8.6 Hz, 1H), 7.57 (dd, J=8.6, 2.0 Hz, 1H), 3.35-3.31 (m, 2H), 2.64 (t, J=7.5 Hz, 2H), 1.80-1.73 (m, 2H), 1.66-1.57 (m, 2H), 1.49-1.35 (m, 4H). ¹³C NMR (151 MHz, acetone-d₆): δ 172.1, 166.4, 158.6, 148.2, 134.3, 129.1, 123.9, 122.6 (q, J=287.2 Hz), 121.2, 115.7, 90.8 (q, J=32 Hz), 39.6, 35.5, 28.7, 28.5, 26.1, 24.7. HRMS-ESI M/z: [M+H]⁺ calcd. 498.0309 for C₁₇H₁₉BrF₃N₃O₄S. found 498.0313, 98% purity.

### N-(6-bromobenzo[d]oxazol-2-yl)-7-(3,3,3-trifluoro-2,2-dihydroxypropanamido)heptanamide (80)

White solid, 15 mg (7.8%) mp. 163-168° C. ¹H NMR (400 MHz, CD₃OD) δ 7.69 (s, 1H), 7.43 (d, J=8.4 Hz, 2H), 3.26 (t, J=7.0 Hz, 2H), 2.52 (t, J=7.2 Hz, 2H), 1.85-1.66 (m, 2H), 1.66-1.51 (m, 2H), 1.5-1.34 (m, 4H). ¹³C NMR (151 MHz, CD₃OD): δ 171.8, 165.2, 156.2, 146.5, 142.6, 126.4, 121.8 (q, J=287.3 Hz), 120.8, 116.9, 111, 94.4 (q, J=32.3 Hz), 39.1, 36, 28.7, 28.3, 26.1, 24.5. HRMS-ESI M/z: [M+H]⁺ calcd. 482.0538 for C₁₇H₁₉BrF₃N₃O₅. found 482.0528, 98% purity.

### N-(benzo[d]oxazol-2-yl)-7-(3,3,3-trifluoro-2,2-dihydroxypropanamido)heptanamide (81)

White solid, 9 mg (5.5%) mp. 118-123° C. ¹H NMR (400 MHz, acetone-d₆): δ 7.67-7.46 (m, 2H), 7.42-7.19 (m, 2H), 3.40-3.32 (m, 2H), 2.69 (q, J=8.3 Hz, 2H), 1.93-1.71 (m, 2H), 1.67-1.55 (m, 2H), 1.57-1.35 (m, 4H). ¹³C NMR (151 MHz, CD₃OD): δ 171.8, 165.2, 155, 147.4, 140.7, 124.2, 123.7, 121.8 (q, J=287.3 Hz), 119, 117.9, 109.5, 94.4 (q, J=32 Hz), 39.2, 36, 28.7, 28.3, 26.1, 24.5. HRMS-ESI M/z: [M+H]⁺ calcd. 404.1433 for C₁₇H₂₀F₃N₃O₅. found 404.1439, 98% purity.

### N-(6-methoxybenzo[d]thiazol-2-yl)-7-(3,3,3-trifluoro-2,2-dihydroxypropanamido)heptanamide (82)

White solid, 17 mg (9.4%) mp. 143-148° C. ¹HNMR (600 MHz, CD₃OD): δ 7.63 (d, J=8.9 Hz, 1H), 7.43 (d, J=2.5 Hz, 1H), 7.04 (dd, J=8.8, 2.5 Hz, 1H), 3.87 (s, 3H), 3.29 (td, J=6.9, 4.2 Hz, 2H), 2.53 (t, J=7.5 Hz, 2H), 1.78-1.71 (m, 2H), 1.62-1.54 (m, 2H), 1.49-1.36 (m, 4H). ¹³C NMR (151 MHz, CD₃OD): 24.8, 26.1, 28.4, 28.7, 35.2, 39.1, 54.8, 94.3 (J=32 Hz), 103.7, 114.8, 120.9, 121.8 (q, J=286 Hz), 133.1, 142.6, 156.5, 157, 165.2, 172.8. HRMS-ESI M/z: [M+H]⁺ calcd. 450.1310 for C₁₈H₂₂F₃N₃O₅S. found 450.1306, 99% purity.

### N-(5-methoxybenzo[d]thiazol-2-yl)-7-(3,3,3-trifluoro-2,2-dihydroxypropanamido)heptanamide (83)

White solid, 14 mg (7.8%) mp. 160-165° C. ¹HNMR (400 MHz, CD₃OD): δ 7.72 (d, J=8.7 Hz, 1H), 7.29 (d, J=2.4 Hz, 1H), 6.96 (dd, J=8.7, 2.4 Hz, 1H), 3.87 (s, 3H), 3.29 (td, J=7.0, 4.1 Hz, 2H), 2.54 (t, J=7.5 Hz, 2H), 1.80-1.71 (m, 2H), 1.63-1.55 (m, 2H), 1.48-1.37 (m, 4H). ¹³C NMR (151 MHz, CD₃OD): δ 172.8, 165.2, 159.5, 159.3, 149.8, 121.8 (q, J=287.3 Hz), 121.2, 112.8, 103.6, 54.6, 39.1, 35.2, 28.7, 28.4, 26.1, 24.7. HRMS-ESI M/z: [M+H]⁺ calcd. 450.1310 for C₁₈H₂₂F₃N₃O₅S. found 450.1312, 98% purity.

### N-(5-(4-methoxyphenyl)thiazol-2-yl)-7-(3,3,3-trifluoro-2,2-dihydroxypropanamido)heptanamide (84)

White solid 18 mg (9.4%) mp. 148-153° C. ¹H NMR (400 MHz, acetone-d₆): δ 7.86 (d, J=8.9 Hz, 2H), 7.30 (s, 1H), 6.98 (d, J=8.9 Hz, 2H), 3.85 (s, 3H), 3.34 (q, J=6.9 Hz, 2H), 2.62 (t, J=7.4 Hz, 2H), 1.83-1.72 (m, 2H), 1.67-1.56 (m, 2H), 1.51-1.36 (m, 4H). ¹³C NMR (151 MHz, acetone-d₆): δ 171.1, 166.4, 159.5, 157.8, 149.4, 127.7, 127.1, 122.6 (q, J=287.2 Hz), 113.9, 105.2, 90.8 (q, J=32.5 Hz), 54.7, 39.6, 35.3, 26.2, 24.9. HRMS-ESI M/z: [M+H]⁺ calcd. 476.1361 for C₂₀H₂₄F₃N₃O₅S. found 476.1368, 99% purity.

### N-(5-(4-bromophenyl)thiazol-2-yl)-7-(3,3,3-trifluoro-2,2-dihydroxypropanamido)heptanamide (85)

White solid, 10 mg (4.8%) mp. 145-150° C. ¹H NMR (600 MHz, acetone-d₆): δ 7.83 (d, J=8.4 Hz, 2H), 7.54 (d, J=8.3 Hz, 2H), 7.49 (s, 1H), 3.27 (q, J=5.4 Hz, 2H), 2.56 (t, J=7.5 Hz, 2H), 1.75-1.67 (m, 2H), 1.60-1.52 (m, 2H), 1.43-1.34 (m, 4H). ¹³C NMR (151 MHz, acetone-d₆): δ 171.3, 166.4, 158.2, 148.2, 134, 131.6, 127.7, 122.6 (q, J=287.4 Hz), 121, 108, 90.8 (q, J=32 Hz), 39.6, 35.3, 26.2, 24.9. HRMS-ESI M/z: [M+H]⁺ calcd. 524.0466 for C₁₉H₂₁BrF₃N₃O₄S. found 524.046, 99% purity.

### Synthesis of 86

A mixture of 7-((5-(4-hydroxyphenyl)thiazol-2-yl) amino)-7-oxoheptan-1-aminium chloride 51 (62 mg, 0.174 mmol), trifluoropyruvic acid (27 mg, 0.17 mmol, 0.98 equiv.), HATU (65 mg, 0.17 mmol, 0.98 equiv.), HOBt (23 mg, 0.17 mmol, 0.98 equiv.), and NMM (56 μL, 0.51 mmol, 2.93 equiv.) was stirred in 1 mL dimethylformamide at room temperature overnight. Ethyl acetate was added to the mixture (5 ml), and the mixture was washed several times with ice water (3 ml). The organic layer was dried (Na₂SO₄), the solvent was removed under reduced pressure, and the mixture was dissolved in silica and purified by flash chromatography (MeOH/DCM 1.5-2%) to yield N-(5-(4-hydroxyphenyl) thiazol-2-yl)-7-(3,3,3-trifluoro-2,2-dihydroxypropanamido) heptanamide 86 as a white solid (10 mg, 11%, mp. 145-150° C.). ¹H NMR (400 MHz, acetone-d₆) δ 7.73 (d, J=8.8 Hz, 2H), 7.19 (s, 1H), 6.84 (d, J=8.8 Hz, 2H), 3.30 (q, J=13.4, 6.7 Hz, 2H), 2.57 (t, J=7.4 Hz, 2H), 1.77-1.68 (m, 2H), 1.61-1.52 (m, 2H), 1.50-1.33 (m, 4H). ¹³C NMR (151 MHz, acetone-d₆): 171.1, 166.4, 157.7, 157.3, 149.7, 127.2, 126.7, 122.6 (q, J=287.8 Hz), 115.3, 104.6, 39.6, 35.3, 26.2, 24.9. HRMS-ESI M/z: [M+H]⁺ calcd. 462.1310 for C₁₉H₂₂F₃N₃O₅S. found 462.1329, 98% purity.

### Synthesis of 87

A mixture of tert-butyl (7-((5-(4-hydroxyphenyl) thiazol-2-yl) amino)-7-oxoheptyl) carbamate 52 (183 mg, 0.44 mmol), 1,8-diazabicyclo[5.4.0]undec-7-ene (75 μL, 0.5 mmol, 1.14 equiv), and ethyl iodide (160 μL, 2 mmol, 4.55 equiv) was refluxed in acetone (3 mL) for 6 hours, after which the solvent was removed under reduced pressure. The reaction mixture was diluted with ethyl acetate (5 mL) and washed with 1M HCl (5 mL) and brine (2×5 mL) twice. The organic layer was dried (Na₂SO₄), concentrated, dried under vacuum, and used in the next reaction without further purification. To the crude mixture was added 20% trifluoroacetic acid in dichloromethane (1 ml: 4 ml), and the mixture was stirred at room temperature for four hours. The solvent was removed under reduced pressure, and the mixture was dried under vacuum, followed by addition of N,N-dimethyl formamide (1 ml), trifluoro pyruvic acid (64 mg, 0.4 mmol, 1 equiv), HATU (152 mg, 0.4 mmol, equiv,), HOBt (54 mg, 0.4 mmol, 1 equiv), and NMM (132 μl, 1.2 mmol, 3 equiv). The mixture was stirred for four hours, ethyl acetate was added to the mixture (5 ml), and the mixture was washed several times with ice water (3 ml). The organic layer was dried (Na₂SO₄), the solvent was removed under reduced pressure, and the mixture was dissolved in silica and purified by flash chromatography (MeOH/DCM 1.5-2%) to yield N-(5-(4-ethoxyphenyl) thiazol-2-yl)-7-(3,3,3-trifluoro-2,2-dihydroxypropanamido)heptanamide 87 as a white solid (10 mg, 4.66%, mp. 138-143° C., 3 steps).

¹H NMR (600 MHz, Acetone-d₆): ¹H NMR (600 MHz, Acetone) δ 7.79 (d, J=8.8 Hz, 23H), 7.25 (s, 1H), 6.91 (d, J=8.8 Hz, 2H), 4.04 (q, J=7.0 Hz, 2H), 3.28 (q, J=7.1 Hz, 2H), 2.56 (t, J=7.5 Hz, 2H), 1.76-1.66 (m, 2H), 1.62-1.52 (m, 2H), 1.42-1.36 (m, 4H), 1.34 (t, J=7.0 Hz, 3H). ¹³C NMR (151 MHz, CD₃OD): 172.4, 165.2, 158.9, 157.9, 149.7, 129.9, 127.4, 126.9, 121.8 (q, J=287.3 Hz), 115.1, 105.1, 94.3 (q, J=32.3 Hz), 63.1, 39.1, 35.1, 28.6, 28.4, 26.1, 24.9, 13.8. HRMS-ESI M/z: [M+H]⁺ calcd. 490.1623 for C₂₁H₂₆F₃N₃O₅S. found 490.1623, 98% purity.

### Biology

### Cell Culture and Cell Viability Assays for Cancer and Normal Cell Lines

HCT116 human colon cancer cells, NCI-H522 human lung cancer cells, HT1080 fibrosarcoma cells, HeLa cervical cancer cells, U2OS osteosarcoma cells, MDA-MB-231 and MDA-MB-468 breast cancer cells, WI38 diploid human cell line, and human retinal pigment epithelial RPE cells were preserved in Dulbecco's Modified Eagle's medium (Mediatech, Inc.) supplemented with 10% calf serum (Atlanta Biologicals) or 10% fetal bovine serum (Gemini Bio-Products #100-106) and 1000 U/ml of both Penicillin and Streptomycin (Mediatech, Inc.) at 37° C. with 5% CO₂. Cell feasibility was examined using methylene blue staining. Cells were plated at a rate of 5000/well in 96 well plates (n=3) respectively and treated the next day. Three days after treatment, cells were fixed/stained in methylene blue saturated in 50% ethanol for 30 min at room temperature. Excessive water was used to wash off extra dye from the plates. The retained dye in the plates was dissolved in 0.1 N HCl and absorbance was measured at 668 nm.

### Western Blotting

NCI-H522 cell pellets obtained were lysed using lysis buffer containing 50 mM Tris (pH 7.4), 150 mM NaCl, 0.5% NP-40, (supplemented with 1 μg/ml aprotinin, 2 μg/ml leupeptin, 1 μg/ml pepstatin A, 1 mM DTT, and 0.1 M PMSF) for 30 minutes on ice and centrifuged at 13×10³ g for 25 minutes at 4° C. The protein levels of the obtained lysates were normalized using BCA Protein Assay Kit (Pierce) and separated by SDS-polyacrylamide gel electrophoresis (12.5% acrylamide). Transfer to polyvinylidene difluoride membranes (Millipore) was followed by obstruction of membranes with blocking buffer containing 5% (w/v) non-fat dry milk dissolved in PBST [1×PBS containing 0.05% (v/v) Tween 20] for 1 hour at room temperature. Membranes were then incubated with corresponding primary antibodies overnight at 4° C. The membranes were then washed (3×15 min each) with PBST and incubated with secondary antibodies conjugated to horse-radish peroxidase, obtained from Biorad, and used at a dilution of 1:10,000. Bound antibodies were detected using enhanced chemiluminescence (Biorad).

Inhibitor Testing with HDAC Isoforms

In a half-area 96-well white opaque plate (Corning), recombinant HDAC1 (1 μL of 3 ng/L, BPS Bioscience), HDAC2 (1 μL of 1 ng/L, BPS Bioscience), HDAC3 (1 μL of 30 ng/L, BPS Bioscience), HDAC4 (1 μL of 5 ng/L, BPS Bioscience), HDAC6 (1 μL of 35 ng/&L, BPS Bioscience), HDAC8 (1 μL of 70 ng/&L, BPS Bioscience), or buffer alone (1 μL) was added to HDAC-Glo™ buffer (43 μL, Promega). Serial dilutions or single concentrations of inhibitors (1 μL in DMSO, concentrations described in the supporting information) or DMSO alone (1 μL) were added to the enzyme solution, followed by 3 hr. incubation at room temperature. The HDAC-Glo™ reagent (5 μL), prepared per manufacturer recommendations, was added to each reaction. Luminescent signal was measured every 3 min over the course of 30 min using an M-Plex Infinite 200 Pro (Tecan). To determine IC₅₀ values, the luminescent signal at peak reading was first background corrected with the signal from a background control reaction without HDAC enzyme. Percent deacetylase activity values were calculated by dividing the background-corrected signal with inhibitor by the background-corrected signal without inhibitor. IC₅₀ values were calculated by fitting the percent deacetylase activity remaining as a function of inhibitor concentration to a sigmoidal dose-response curve (y=100/(1+(x/IC₅₀)^{z}), where y=percent deacetylase activity and x=inhibitor concentration) using non-linear regression with KaleidaGraph 5.0 software.

### S9 Fraction Assay

A protocol similar to the one previously employed by others using Human Liver S9 fractions instead of liver microsomes was used for the metabolism studies. The assay was initiated by incubating a mixture of the substrate (0.02 mM), buffer (0.1M), and NADPH (1 mM) at 37° C. for ten minutes, followed by addition of the S9 fraction (0.5 mg) to the mixture and gentle mixing. The total volume of the reaction mixture was maintained at 1 mL. 450 μL aliquots of the mixture were collected at different time intervals (1h, 2 hrs) for compound 60 and 200 μL aliquots every 15 minutes for the TFMK 91, as it has a half-life of less than 30 minutes. The reactions in the aliquots were terminated by adding an equal volume of ethyl acetate, mixing thoroughly, and transferring into micro centrifuge tubes. They were vortexed for 2 minutes and then subjected to centrifugation at 10,000×g for 15 minutes to remove the proteins. The top organic layers were extracted and evaporated under reduced pressure. The organic residues from the aliquots were dissolved in methanol first, then diluted 1:1 with water, and analyzed using LC-ESI-MS. A 450 μL aliquot of compound 60 with concentration of 0.02 mM contains 9 nanomoles, which after evaporation, was dissolved in 500 μL of methanol to give 18 μM in concentration of 60. This was diluted 1:1 with water to give a final concentration of 9 μM, which was used for LC-ESI-MS. Similarly, 200 μL of TFMK 91 at a concentration of 0.02 mM contains 4 nanomoles, which after removing ethyl acetate, and dissolving in 250 μL of methanol gives 16 μM concentration of 91. This was diluted 1:1 with water to give 8 μM final concentration for LC-ESI-MS.

### LC-ESI-MS Protocol

For analysis of the aliquots obtained, the LC-ESI-MS protocol shown in Table 6 was used. The solvent flow rate was 0.2 mL/min, and the sample injection volume was 20 μL. Mobile phase A consisted of water containing 0.1% formic acid, while mobile phase B was acetonitrile containing 0.1% formic acid. The column was equilibrated with 30% of mobile phase A and 70% of mobile phase B. The retention times for compound 60, compound 92 (TFMHA), and compound 91 (TFMK) were 7.5 min (t 0.1 min), 8.13 min, and 10.5 min (i 0.3 min), respectively. At the start, the parameters for both starting materials were established. The M+1 for compound 60 was m/z 370 (hydrate) with a small percentage having an M+1 of m/z 352 m/z (ketone). Its MS/MS peak gave only m/z 352. The compound 91 had M+1 peaks of m/z 302 (ketone), m/z 320 (hydrate), and another peak of m/z 338 corresponding to a dihydrate, while the MS/MS corresponded to m/z 302 with a lesser amount of m/z 320. The TFMHA 92 had an M+1 peak of 354 m/z (13C).

Certain embodiments of the compositions and methods disclosed herein are defined in the above examples. It should be understood that these examples, while indicating particular embodiments of the invention, are given by way of illustration only. From the above discussion and these examples, one skilled in the art can ascertain the essential characteristics of this disclosure, and without departing from the spirit and scope thereof, can make various changes and modifications to adapt the compositions and methods described herein to various usages and conditions. Various changes may be made and equivalents may be substituted for elements thereof without departing from the essential scope of the disclosure. In addition, many modifications may be made to adapt a particular situation or material to the teachings of the disclosure without departing from the essential scope thereof.

What is claimed is:

1. A composition comprising an HDAC inhibitor compound having a cap group, a linker, and a zinc binding group, wherein the zinc binding group comprises a trifluoromethylpyruvamide (TFMP) or a hydrate thereof.

2. A composition comprising Formula I: 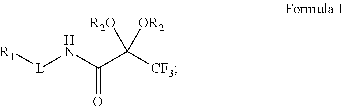
wherein:
R₁ is aryl, alkyl, heteroaryl, a hydrophobic moiety, a hydrophilic moiety, or a combination thereof, optionally substituted with one or more halogens, nitro groups, or amino groups;
each R₂ is, independently, H or alkyl; and
L is a linker comprising an alkyl chain, a fluorinated alkyl chain, an aryl group, an arylalkyl group, a carbonyl-containing chain, an amide-containing chain, or a heteroatom-containing chain, optionally substituted with one or more halogens, amino groups, or carboxylic acid groups;
and salts, stereoisomers, racemates, solvates, hydrates, prodrugs, and polymorphs thereof.

3. The composition of claim 2, wherein:
R₁ comprises a benzyl, phenyl, pyridinyl, thiazolyl, or benzothiazolyl; and
L comprises a carbonyl-containing alkyl chain having from 1 to 10 carbons.

4. The composition of claim 2, wherein R₁ comprises a polyethylene glycol (PEG) chain.

5. The composition of claim 2, wherein L comprises an ether, a thioether, an amide, or a carbonyl-containing alkyl chain having from 1 to 10 carbons.

6. The composition of claim 2, wherein the composition comprises compound 83:

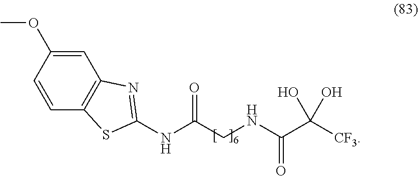

7. The composition of claim 2, wherein the composition comprises compound 84:

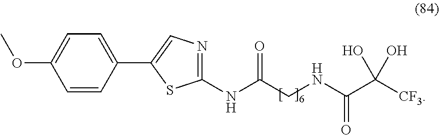

8. The composition of claim 2, wherein the composition comprises compound 85:

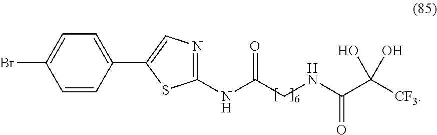

9. The composition of claim 2, wherein the composition comprises compound 86:

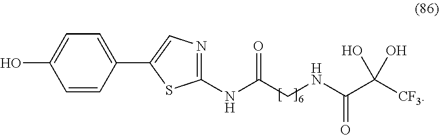

10. The composition of claim 2, wherein the composition comprises compound 61:

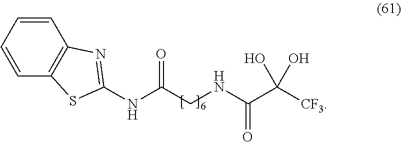

11. The composition of claim 2, wherein the composition comprises compound 58:

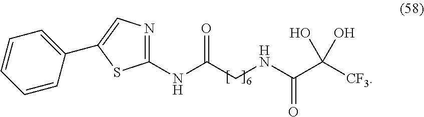

12. The composition of claim 2, wherein each R₂ is H.

13. A pharmaceutical composition comprising:
the composition of claim 2; and
a pharmaceutically acceptable diluent, adjuvant, or carrier.

14. A metal binding group comprising Formula II: 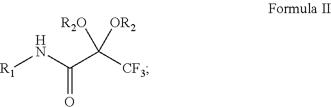
wherein
R₁ is a lone pair of electrons, aryl, alkyl, heteroaryl, a hydrophobic moiety, or a hydrophilic moiety, optionally substituted with one or more halogens, nitro groups, or amino groups; and
each R₂ is, independently, H or alkyl;
and salts stereoisomers, racemates, hydrates, solvates, prodrugs, and polymorphs thereof.

15. The metal binding group of claim 14, wherein each R₂ is H.

16. A method of inhibiting an HDAC protein, the method comprising contacting a cell with a composition of claim 2 and inhibiting an HDAC protein in the cell.

17. The method of claim 16, wherein the HDAC protein is HDAC8 and the composition comprises compound 83.

18. The method of claim 16, wherein the HDAC inhibitor compound is selected from the group consisting of compound 65, compound 61, compound 85, compound 69, compound 55, compound 86, compound 64, compound 83, compound 58, and compound 84.

19. A method of inhibiting growth of cancer cells, the method comprising contacting cancer cells with an effective amount of a composition of claim 2 and inhibiting growth of the cancer cells;
wherein the cancer is lung cancer, cervical cancer, breast cancer, colon cancer, fibrosarcoma, neuroblastoma, or osteosarcoma.

20. The method of claim 19, wherein the HDAC inhibitor compound is selected from the group consisting of compound 65, compound 61, compound 85, compound 69, compound 55, compound 86, compound 64, compound 83, compound 58, and compound 84.
